SEQUENCE LISTING (214 sequences)

SEQ ID NO: 1 - DNA, 2214 bp, Artificial Sequence
  Synthetic Construct
atggactggacttggaggattctgtttctggtcgccgccgctaccgggactcacgctcaggtgcagctggtcgagagtggagggggagtggtgcagccaggcaggagcctgagactgtcctgcgccgcctctggcttcgcctttagcaactacggcatgaattgggtgaggcaggcacctggcaagggcctggagtgggtggccgtgatctggcacgacggctccctgaagtactatacacagtctgtgaagggccggttcaccatctcccgcgacaacgccaagaataccctgtttctgcagatggattctctgagcgccgacgataccgccatgtactattgcacagtgtggtacctggagacacctgacgatggcttcgacatctggggcagaggcaccatggtgacagtgagctccgccagcacaaagggcccatccgtgtttccactggccccctctagcaagtccacctctggcggcacagccgccctgggctgtctggtgaaggattatttcccagagcccgtgaccgtgtcttggaacagcggcgccctgaccagcggagtgcacacatttccagccgtgctgcagtcctctggcctgtactccctgagctccgtggtgacagtgccctctagctccctgggcacccagacatatatctgcaacgtgaatcacaagccaagcaataccaaggtggacaagaaggtggagcccaagtcctgtgataagacccacacatgccctccctgtcctgcaccagagctgctgggcggcccaagcgtgttcctgtttccacccaagcctaaggacacactgatgatctctcggacccccgaggtgacatgcgtggtggtggacgtgagccacgaggaccccgaggtgaagttcaactggtacgtggatggcgtggaggtgcacaatgccaagaccaagcccagggaggagcagtacaactctacatatagagtggtgagcgtgctgaccgtgctgcaccaggactggctgaacggcaaggagtataagtgcaaggtgtccaataaggccctgcccgcccctatcgagaagacaatctctaaggcaaagggacagcctcgggagccacaggtgtacaccctgcctccatcccgcgacgagctgacaaagaaccaggtgtctctgacctgtctggtgaagggcttctatccttctgatatcgccgtggagtgggagagcaatggccagccagagaacaattacaagaccacaccccctgtgctggactccgatggctctttctttctgtatagcaagctgaccgtggataagtcccgctggcagcagggcaacgtgttcagctgttccgtgatgcacgaagccctgcacaaccactacacccagaagagcctgtccctgtcccctggcaaaaggggaaggaagaggagaagcggctccggcgccacaaacttcagcctgctgaagcaggcaggcgatgtggaggagaatccaggacctatggcctggactcccctgctgctgcctctgctgactttttgcactgtctccgaagcctcacagctggtcctgactcagccccctagcgcaagcgcctccctgggcgtgtccgtgacactgacctgcacactgtcccacggccacacctctaaggcaatcgcatggcaccagcagcagccaggcaagggccccaggtacctgatgaaggtgaactccgacggctctcacacaaagggagcagcagtgccagataggttctctggcagcacctccggcgccgagagacactttacaatctctaatctgcagagcgacgatgaggccgactactattgccaggcctgggattctggcatctgggtgttcggcggcggaaccaagctgacagtgctgggacagcctaaggcagcaccaagcgtgaccctgtttccccctagctccgaggagctgcaggccaacaaggccaccctggtgtgcctgatcagcgacttctacccaggagcagtgacagtggcatggaaggccgattctagccccgtgaaggccggcgtggagacaacaacccctagcaagcagtccaacaataagtacgccgcctcctcttatctgagcctgacacccgagcagtggaagtcccacaagtcttatagctgccaggtcacccatgaaggaagcacagtggaaaagacagtcgcaccaacagaatgtagctgataa

SEQ ID NO: 2 - protein, 736 aa, Artificial Sequence
  Synthetic Construct
MDWTWRILFLVAAATGTHAQVQLVESGGGVVQPGRSLRLSCAASGFAFSNYGMNWVRQAPGKGLEWVAVIWHDGSLKYYTQSVKGRFTISRDNAKNTLFLQMDSLSADDTAMYYCTVWYLETPDDGFDIWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMAWTPLLLPLLTFCTVSEASQLVLTQPPSASASLGVSVTLTCTLSHGHTSKAIAWHQQQPGKGPRYLMKVNSDGSHTKGAAVPDRFSGSTSGAERHFTISNLQSDDEADYYCQAWDSGIWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 3 - DNA, 1359 bp, Artificial Sequence
  Synthetic Construct
caggtgcagctggtcgagagtggagggggagtggtgcagccaggcaggagcctgagactgtcctgcgccgcctctggcttcgcctttagcaactacggcatgaattgggtgaggcaggcacctggcaagggcctggagtgggtggccgtgatctggcacgacggctccctgaagtactatacacagtctgtgaagggccggttcaccatctcccgcgacaacgccaagaataccctgtttctgcagatggattctctgagcgccgacgataccgccatgtactattgcacagtgtggtacctggagacacctgacgatggcttcgacatctggggcagaggcaccatggtgacagtgagctccgccagcacaaagggcccatccgtgtttccactggccccctctagcaagtccacctctggcggcacagccgccctgggctgtctggtgaaggattatttcccagagcccgtgaccgtgtcttggaacagcggcgccctgaccagcggagtgcacacatttccagccgtgctgcagtcctctggcctgtactccctgagctccgtggtgacagtgccctctagctccctgggcacccagacatatatctgcaacgtgaatcacaagccaagcaataccaaggtggacaagaaggtggagcccaagtcctgtgataagacccacacatgccctccctgtcctgcaccagagctgctgggcggcccaagcgtgttcctgtttccacccaagcctaaggacacactgatgatctctcggacccccgaggtgacatgcgtggtggtggacgtgagccacgaggaccccgaggtgaagttcaactggtacgtggatggcgtggaggtgcacaatgccaagaccaagcccagggaggagcagtacaactctacatatagagtggtgagcgtgctgaccgtgctgcaccaggactggctgaacggcaaggagtataagtgcaaggtgtccaataaggccctgcccgcccctatcgagaagacaatctctaaggcaaagggacagcctcgggagccacaggtgtacaccctgcctccatcccgcgacgagctgacaaagaaccaggtgtctctgacctgtctggtgaagggcttctatccttctgatatcgccgtggagtgggagagcaatggccagccagagaacaattacaagaccacaccccctgtgctggactccgatggctctttctttctgtatagcaagctgaccgtggataagtcccgctggcagcagggcaacgtgttcagctgttccgtgatgcacgaagccctgcacaaccactacacccagaagagcctgtccctgtcccctggcaaatgataa

SEQ ID NO: 4 - protein, 451 aa, Artificial Sequence
  Synthetic Construct
QVQLVESGGGVVQPGRSLRLSCAASGFAFSNYGMNWVRQAPGKGLEWVAVIWHDGSLKYYTQSVKGRFTISRDNAKNTLFLQMDSLSADDTAMYYCTVWYLETPDDGFDIWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 657 bp, Artificial Sequence
  Synthetic Construct
cagctggtcctgactcagccccctagcgcaagcgcctccctgggcgtgtccgtgacactgacctgcacactgtcccacggccacacctctaaggcaatcgcatggcaccagcagcagccaggcaagggccccaggtacctgatgaaggtgaactccgacggctctcacacaaagggagcagcagtgccagataggttctctggcagcacctccggcgccgagagacactttacaatctctaatctgcagagcgacgatgaggccgactactattgccaggcctgggattctggcatctgggtgttcggcggcggaaccaagctgacagtgctgggacagcctaaggcagcaccaagcgtgaccctgtttccccctagctccgaggagctgcaggccaacaaggccaccctggtgtgcctgatcagcgacttctacccaggagcagtgacagtggcatggaaggccgattctagccccgtgaaggccggcgtggagacaacaacccctagcaagcagtccaacaataagtacgccgcctcctcttatctgagcctgacacccgagcagtggaagtcccacaagtcttatagctgccaggtcacccatgaaggaagcacagtggaaaagacagtcgcaccaacagaatgtagctgataa

SEQ ID NO: 6 - protein, 217 aa, Artificial Sequence
  Synthetic Construct
QLVLTQPPSASASLGVSVTLTCTLSHGHTSKAIAWHQQQPGKGPRYLMKVNSDGSHTKGAAVPDRFSGSTSGAERHFTISNLQSDDEADYYCQAWDSGIWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 7 - DNA, 363 bp, Artificial Sequence
  Synthetic Construct
caggtgcagctggtcgagagtggagggggagtggtgcagccaggcaggagcctgagactgtcctgcgccgcctctggcttcgcctttagcaactacggcatgaattgggtgaggcaggcacctggcaagggcctggagtgggtggccgtgatctggcacgacggctccctgaagtactatacacagtctgtgaagggccggttcaccatctcccgcgacaacgccaagaataccctgtttctgcagatggattctctgagcgccgacgataccgccatgtactattgcacagtgtggtacctggagacacctgacgatggcttcgacatctggggcagaggcaccatggtgacagtgagctcc

SEQ ID NO: 8 - protein, 121 aa, Artificial Sequence
  Synthetic Construct
QVQLVESGGGVVQPGRSLRLSCAASGFAFSNYGMNWVRQAPGKGLEWVAVIWHDGSLKYYTQSVKGRFTISRDNAKNTLFLQMDSLSADDTAMYYCTVWYLETPDDGFDIWGRGTMVTVSS

SEQ ID NO: 9 - DNA, 333 bp, Artificial Sequence
  Synthetic Construct
cagctggtcctgactcagccccctagcgcaagcgcctccctgggcgtgtccgtgacactgacctgcacactgtcccacggccacacctctaaggcaatcgcatggcaccagcagcagccaggcaagggccccaggtacctgatgaaggtgaactccgacggctctcacacaaagggagcagcagtgccagataggttctctggcagcacctccggcgccgagagacactttacaatctctaatctgcagagcgacgatgaggccgactactattgccaggcctgggattctggcatctgggtgttcggcggcggaaccaagctgacagtgctg

SEQ ID NO: 10 - protein, 111 aa, Artificial Sequence
  Synthetic Construct
QLVLTQPPSASASLGVSVTLTCTLSHGHTSKAIAWHQQQPGKGPRYLMKVNSDGSHTKGAAVPDRFSGSTSGAERHFTISNLQSDDEADYYCQAWDSGIWVFGGGTKLTVL

SEQ ID NO: 11 - DNA, 996 bp, Artificial Sequence
  Synthetic Construct
gccagcacaaagggcccatccgtgtttccactggccccctctagcaagtccacctctggcggcacagccgccctgggctgtctggtgaaggattatttcccagagcccgtgaccgtgtcttggaacagcggcgccctgaccagcggagtgcacacatttccagccgtgctgcagtcctctggcctgtactccctgagctccgtggtgacagtgccctctagctccctgggcacccagacatatatctgcaacgtgaatcacaagccaagcaataccaaggtggacaagaaggtggagcccaagtcctgtgataagacccacacatgccctccctgtcctgcaccagagctgctgggcggcccaagcgtgttcctgtttccacccaagcctaaggacacactgatgatctctcggacccccgaggtgacatgcgtggtggtggacgtgagccacgaggaccccgaggtgaagttcaactggtacgtggatggcgtggaggtgcacaatgccaagaccaagcccagggaggagcagtacaactctacatatagagtggtgagcgtgctgaccgtgctgcaccaggactggctgaacggcaaggagtataagtgcaaggtgtccaataaggccctgcccgcccctatcgagaagacaatctctaaggcaaagggacagcctcgggagccacaggtgtacaccctgcctccatcccgcgacgagctgacaaagaaccaggtgtctctgacctgtctggtgaagggcttctatccttctgatatcgccgtggagtgggagagcaatggccagccagagaacaattacaagaccacaccccctgtgctggactccgatggctctttctttctgtatagcaagctgaccgtggataagtcccgctggcagcagggcaacgtgttcagctgttccgtgatgcacgaagccctgcacaaccactacacccagaagagcctgtccctgtcccctggcaaatgataa

SEQ ID NO: 12 - protein, 330 aa, Artificial Sequence [sequence omitted]

SEQ ID NO: 13 - DNA, 324 bp, Artificial Sequence
  Synthetic Construct
ggacagcctaaggcagcaccaagcgtgaccctgtttccccctagctccgaggagctgcaggccaacaaggccaccctggtgtgcctgatcagcgacttctacccaggagcagtgacagtggcatggaaggccgattctagccccgtgaaggccggcgtggagacaacaacccctagcaagcagtccaacaataagtacgccgcctcctcttatctgagcctgacacccgagcagtggaagtcccacaagtcttatagctgccaggtcacccatgaaggaagcacagtggaaaagacagtcgcaccaacagaatgtagctgataa

SEQ ID NO: 14 - protein, 106 aa, Artificial Sequence
  Synthetic Construct
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 15 - DNA, 2216 bp, Artificial Sequence
  Synthetic Construct
atggactggacttggaggattctgtttctggtcgccgccgcaactgggactcacgctcaggtgcagctggtcgaatcaggaggaggggtggtgcagcccggcaggtccctgagactgtcttgcgccgccagcggcttcgcctttagcaactacggaatgcactgggtgaggcaggcacctggcaagggcctggagtgggtggccgtgatctggcacgacggctccctgaagtactatacacagtctgtgaagggccggttcaccatctcccgcgacaactctaagaataccctgtacctgcagatgaattccctgagagccgaggataccgccgtgtactattgcacagtgtggtacctggagacacctgacgatggcttcgacatctggggccagggcaccatggtgacagtgagctccgccagcacaaagggcccatccgtgtttccactggccccctctagcaagtctaccagcggcggcacagccgccctgggatgtctggtgaaggattacttcccagagccagtgaccgtgagctggaactccggcgccctgaccagcggagtgcacacatttccagccgtgctgcagtcctctggcctgtactccctgagctccgtggtgacagtgccctctagctccctgggcacccagacatatatctgcaacgtgaatcacaagccaagcaataccaaggtggacaagaaggtggagcccaagtcctgtgataagacccacacatgccctccctgtcctgcaccagagctgctgggcggcccaagcgtgttcctgtttccacccaagcctaaggacacactgatgatctctcggacccccgaggtgacatgcgtggtggtggacgtgagccacgaggaccccgaggtgaagtttaactggtacgtggatggcgtggaggtgcacaatgccaagaccaagcccagggaggagcagtacaactctacatatagagtggtgagcgtgctgaccgtgctgcaccaggactggctgaacggcaaggagtataagtgcaaggtgtccaataaggccctgcccgcccctatcgagaagacaatctctaaggcaaagggacagcctcgggagccacaggtgtacaccctgcctccatcccgcgacgagctgacaaagaaccaggtgtctctgacctgtctggtgaagggcttctatccttctgatatcgccgtggagtgggagagcaatggccagccagagaacaattacaagaccacaccccctgtgctggactctgatggcagcttctttctgtatagcaagctgaccgtggataagtcccgctggcagcagggcaacgtgttcagctgttctgtgatgcacgaagcactgcacaaccactacacccagaagtcactgtcactgtcaccagggaagtgaggggaaggaagaggagaagcggctccggcgccacaaacttcagcctgctgaagcaggcaggcgatgtggaggagaatccaggacctatggcctggactcccctgctgctgcctctgctgactttttgcactgtctccgaagcctcacagctggtcctgactcagccccctagcgcaagcgcctccctgggcgtgtccgtgacactgacctgcacactgtcccacggccacacctctaaggcaatcgcatggcaccagcagcagccaggcaagggccccaggtacctgatgaaggtgaactccgacggctctcacacaaagggagcagcagtgccagataggttctctggcagcacctccggcgccgagagacactttacaatctctaatctgcagagcgacgatgaggccgactactattgccaggcctgggattctggcatctgggtgttcggcggcggaaccaagctgacagtgctgggacagcctaaggcagcaccaagcgtgaccctgtttccccctagctccgaggagctgcaggccaacaaggccaccctggtgtgcctgatcagcgacttctacccaggagcagtgacagtggcatggaaggccgattctagccccgtgaaggccggcgtggagacaacaacccctagcaagcagtccaacaataagtacgccgcctcctcttatctgagcctgacacccgagcagtggaagtcccacaagtcttatagctgccaggtcacccatgaaggaagcacagtggaaaagacagtcgcaccaacagaatgtagctgataa

SEQ ID NO: 16 - protein, 736 aa, Artificial Sequence
  Synthetic Construct
MDWTWRILFLVAAATGTHAQVQLVESGGGVVQPGRSLRLSCAASGFAFSNYGMHWVRQAPGKGLEWVAVIWHDGSLKYYTQSVKGRFTISRDNSKNTLYLQMNSLRAEDTAVYYCTVWYLETPDDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMAWTPLLLPLLTFCTVSEASQLVLTQPPSASASLGVSVTLTCTLSHGHTSKAIAWHQQQPGKGPRYLMKVNSDGSHTKGAAVPDRFSGSTSGAERHFTISNLQSDDEADYYCQAWDSGIWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 17 - DNA, 1359 bp, Artificial Sequence
  Synthetic Construct
caggtgcagctggtcgaatcaggaggaggggtggtgcagcccggcaggtccctgagactgtcttgcgccgccagcggcttcgcctttagcaactacggaatgcactgggtgaggcaggcacctggcaagggcctggagtgggtggccgtgatctggcacgacggctccctgaagtactatacacagtctgtgaagggccggttcaccatctcccgcgacaactctaagaataccctgtacctgcagatgaattccctgagagccgaggataccgccgtgtactattgcacagtgtggtacctggagacacctgacgatggcttcgacatctggggccagggcaccatggtgacagtgagctccgccagcacaaagggcccatccgtgtttccactggccccctctagcaagtctaccagcggcggcacagccgccctgggatgtctggtgaaggattacttcccagagccagtgaccgtgagctggaactccggcgccctgaccagcggagtgcacacatttccagccgtgctgcagtcctctggcctgtactccctgagctccgtggtgacagtgccctctagctccctgggcacccagacatatatctgcaacgtgaatcacaagccaagcaataccaaggtggacaagaaggtggagcccaagtcctgtgataagacccacacatgccctccctgtcctgcaccagagctgctgggcggcccaagcgtgttcctgtttccacccaagcctaaggacacactgatgatctctcggacccccgaggtgacatgcgtggtggtggacgtgagccacgaggaccccgaggtgaagtttaactggtacgtggatggcgtggaggtgcacaatgccaagaccaagcccagggaggagcagtacaactctacatatagagtggtgagcgtgctgaccgtgctgcaccaggactggctgaacggcaaggagtataagtgcaaggtgtccaataaggccctgcccgcccctatcgagaagacaatctctaaggcaaagggacagcctcgggagccacaggtgtacaccctgcctccatcccgcgacgagctgacaaagaaccaggtgtctctgacctgtctggtgaagggcttctatccttctgatatcgccgtggagtgggagagcaatggccagccagagaacaattacaagaccacaccccctgtgctggactctgatggcagcttctttctgtatagcaagctgaccgtggataagtcccgctggcagcagggcaacgtgttcagctgttctgtgatgcacgaagcactgcacaaccactacacccagaagtcactgtcactgtcaccagggaagtgataa

SEQ ID NO: 18 - protein, 451 aa, Artificial Sequence
  Synthetic Construct
QVQLVESGGGVVQPGRSLRLSCAASGFAFSNYGMHWVRQAPGKGLEWVAVIWHDGSLKYYTQSVKGRFTISRDNSKNTLYLQMNSLRAEDTAVYYCTVWYLETPDDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 657 bp, Artificial Sequence; identical to SEQ ID NO: 5
  Synthetic Construct

SEQ ID NO: 20 - protein, 217 aa, Artificial Sequence; identical to SEQ ID NO: 6
  Synthetic Construct

SEQ ID NO: 21 - DNA, 363 bp, Artificial Sequence
  Synthetic Construct
caggtgcagctggtcgaatcaggaggaggggtggtgcagcccggcaggtccctgagactgtcttgcgccgccagcggcttcgcctttagcaactacggaatgcactgggtgaggcaggcacctggcaagggcctggagtgggtggccgtgatctggcacgacggctccctgaagtactatacacagtctgtgaagggccggttcaccatctcccgcgacaactctaagaataccctgtacctgcagatgaattccctgagagccgaggataccgccgtgtactattgcacagtgtggtacctggagacacctgacgatggcttcgacatctggggccagggcaccatggtgacagtgagctcc

SEQ ID NO: 22 - protein, 121 aa, Artificial Sequence
  Synthetic Construct
QVQLVESGGGVVQPGRSLRLSCAASGFAFSNYGMHWVRQAPGKGLEWVAVIWHDGSLKYYTQSVKGRFTISRDNSKNTLYLQMNSLRAEDTAVYYCTVWYLETPDDGFDIWGQGTMVTVSS

SEQ ID NO: 23 - DNA, 2195 bp, Artificial Sequence
  Synthetic Construct
atggactggacttggaggattctgtttctggtcgccgccgcaactgggactcacgctcaggtgcagctggtcgaatcaggaggaggggtggtgcagcccggcaggtccctgagactgtcttgcgccgccagcggcttcgcctttagcaactacggaatgcactgggtgaggcaggcacctggcaagggcctggagtgggtggccgtgatctggcacgacggctccctgaagtactatacacagtctgtgaagggccggttcaccatctcccgcgacaactctaagaataccctgtacctgcagatgaattccctgagagccgaggataccgccgtgtactattgcacagtgtggtacctggagacacctgacgatggcttcgacatctggggccagggcaccatggtgacagtgagctccgccagcacaaagggcccatccgtgtttccactggccccctctagcaagtctaccagcggcggcacagccgccctgggatgtctggtgaaggattacttcccagagccagtgaccgtgagctggaactccggcgccctgaccagcggagtgcacacatttccagccgtgctgcagtcctctggcctgtactccctgagctccgtggtgacagtgccctctagctccctgggcacccagacatatatctgcaacgtgaatcacaagccaagcaataccaaggtggacaagaaggtggagcccaagtcctgtgataagacccacacatgccctccctgtcctgcaccagagctgctgggcggcccaagcgtgttcctgtttccacccaagcctaaggacacactgatgatctctcggacccccgaggtgacatgcgtggtggtggacgtgagccacgaggaccccgaggtgaagtttaactggtacgtggatggcgtggaggtgcacaatgccaagaccaagcccagggaggagcagtacaactctacatatagagtggtgagcgtgctgaccgtgctgcaccaggactggctgaacggcaaggagtataagtgcaaggtgtccaataaggccctgcccgcccctatcgagaagacaatctctaaggcaaagggacagcctcgggagccacaggtgtacaccctgcctccatcccgcgacgagctgacaaagaaccaggtgtctctgacctgtctggtgaagggcttctatccttctgatatcgccgtggagtgggagagcaatggccagccagagaacaattacaagaccacaccccctgtgctggactctgatggcagcttctttctgtatagcaagctgaccgtggataagtcccgctggcagcagggcaacgtgttcagctgttctgtgatgcacgaagcactgcacaaccactacacccagaagtcactgtcactgtcaccagggaagtgggctccggcgccacaaacttcagcctgctgaagcaggcaggcgatgtggaggagaatccaggacctatggcatggacacccctgctgctgcccctgctgactttttgcaccgtctccgaagcctcacagctggtcctgacccagagcccctccgcaagcgcctccctgggagcatccgtgaagctgacatgcaccctgtcccacggccacacatctaaggccatcgcctggcaccagcagcagccagagaagggccccaggtacctgatgaaggtgaactccgacggctctcacacaaagggcgacggcatccccgataggttttctggcagctcctctggcgccgagagatatctgaccatctctaatctgcagagcgaggacgaggccgattactattgccaggcctgggattctggcatctgggtgttcggcggcggaacaaagctgaccgtgctgggacagcctaaggcagcaccaagcgtgacactgtttccccctagctccgaggagctgcaggccaacaaggccaccctggtgtgcctgatctccgacttctacccaggagcagtgaccgtggcatggaaggccgattctagccccgtgaaggccggcgtggagacaacaacccctagcaagcagtccaacaataagtacgccgcctcctcttatctgagcctgacccccgagcagtggaagtcccacaagtcttatagctgtcaggtcacccatgaaggaagcacagtggagaaaaccgtggcaccaaccgaatgtagctgataa

SEQ ID NO: 24 - protein, 736 aa, Artificial Sequence
  Synthetic Construct
MDWTWRILFLVAAATGTHAQVQLVESGGGVVQPGRSLRLSCAASGFAFSNYGMHWVRQAPGKGLEWVAVIWHDGSLKYYTQSVKGRFTISRDNSKNTLYLQMNSLRAEDTAVYYCTVWYLETPDDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMAWTPLLLPLLTFCTVSEASQLVLTQSPSASASLGASVKLTCTLSHGHTSKAIAWHQQQPEKGPRYLMKVNSDGSHTKGDGIPDRFSGSSSGAERYLTISNLQSEDEADYYCQAWDSGIWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 25 - DNA, 1359 bp, Artificial Sequence; identical to SEQ ID NO: 17
  Synthetic Construct

SEQ ID NO: 26 - protein, 451 aa, Artificial Sequence; identical to SEQ ID NO: 18
  Synthetic Construct

SEQ ID NO: 27 - DNA, 657 bp, Artificial Sequence
  Synthetic Construct
cagctggtcctgacccagagcccctccgcaagcgcctccctgggagcatccgtgaagctgacatgcaccctgtcccacggccacacatctaaggccatcgcctggcaccagcagcagccagagaagggccccaggtacctgatgaaggtgaactccgacggctctcacacaaagggcgacggcatccccgataggttttctggcagctcctctggcgccgagagatatctgaccatctctaatctgcagagcgaggacgaggccgattactattgccaggcctgggattctggcatctgggtgttcggcggcggaacaaagctgaccgtgctgggacagcctaaggcagcaccaagcgtgacactgtttccccctagctccgaggagctgcaggccaacaaggccaccctggtgtgcctgatctccgacttctacccaggagcagtgaccgtggcatggaaggccgattctagccccgtgaaggccggcgtggagacaacaacccctagcaagcagtccaacaataagtacgccgcctcctcttatctgagcctgacccccgagcagtggaagtcccacaagtcttatagctgtcaggtcacccatgaaggaagcacagtggagaaaaccgtggcaccaaccgaatgtagctgataa

SEQ ID NO: 28 - protein, 217 aa, Artificial Sequence
  Synthetic Construct
QLVLTQSPSASASLGASVKLTCTLSHGHTSKAIAWHQQQPEKGPRYLMKVNSDGSHTKGDGIPDRFSGSSSGAERYLTISNLQSEDEADYYCQAWDSGIWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 29 - DNA, 333 bp, Artificial Sequence
  Synthetic Construct
cagctggtcctgacccagagcccctccgcaagcgcctccctgggagcatccgtgaagctgacatgcaccctgtcccacggccacacatctaaggccatcgcctggcaccagcagcagccagagaagggccccaggtacctgatgaaggtgaactccgacggctctcacacaaagggcgacggcatccccgataggttttctggcagctcctctggcgccgagagatatctgaccatctctaatctgcagagcgaggacgaggccgattactattgccaggcctgggattctggcatctgggtgttcggcggcggaacaaagctgaccgtgctg

SEQ ID NO: 30 - protein, 111 aa, Artificial Sequence
  Synthetic Construct
QLVLTQSPSASASLGASVKLTCTLSHGHTSKAIAWHQQQPEKGPRYLMKVNSDGSHTKGDGIPDRFSGSSSGAERYLTISNLQSEDEADYYCQAWDSGIWVFGGGTKLTVL

SEQ ID NO: 31 - DNA, 2223 bp, Artificial Sequence
  Synthetic Construct
atggattggacatggagaatcctgttcctggtcgccgccgcaactgggactcacgccgaagtgcagctggtgcagtctggagccgaggtgaagaagcctggcgcctctgtgaaggtgagctgcaaggcctccggctacaccttcacaagctatgcaatccactgggtgaggcaggcaccaggacagagactggagtggatgggctggatcaaggccggcaacggcaatacaaggtacagccagaagtttcaggacagagtgaccatcacaagggacaccagcgcctccacagcctatatggagctgagctccctgcggtccgaggataccgccgtgtactattgcgccctgctgaccgtgctgacaccagacgatgccttcgacatctggggccagggcaccatggtgacagtgtctagcgccagcacaaagggaccatccgtgtttccactggcaccctcctctaagtctaccagcggcggcacagccgccctgggatgtctggtgaaggattacttccccgagcctgtgaccgtgtcttggaacagcggcgccctgaccagcggagtgcacacatttccagccgtgctgcagagctccggcctgtactccctgtctagcgtggtgaccgtgccctcctctagcctgggcacccagacatatatctgcaacgtgaatcacaagccatctaatacaaaggtggacaagaaggtggagcccaagagctgtgataagacccacacatgccctccctgtccagcaccagagctgctgggcggcccatccgtgttcctgtttccacccaagcctaaggacaccctgatgatctcccgcaccccagaggtgacatgcgtggtggtggacgtgtctcacgaggaccccgaggtgaagttcaactggtacgtggatggcgtggaggtgcacaatgccaagaccaagccacgggaggagcagtacaactctacctatcgcgtggtgagcgtgctgacagtgctgcaccaggactggctgaacggcaaggagtataagtgcaaggtgagcaataaggccctgcctgccccaatcgagaagaccatctccaaggccaagggccagcctagggagccacaggtgtacacactgcctccaagcagagacgagctgaccaagaaccaggtgtccctgacatgtctggtgaagggcttctatccttccgatatcgccgtggagtgggagtctaatggccagccagagaacaattacaagaccacaccccctgtgctggactccgatggctctttctttctgtatagcaagctgaccgtggacaagtccaggtggcagcagggcaacgtgttttcctgctctgtgatgcacgaggccctgcacaatcactacacccagaagagcctgtccctgtctccaggcaagaggggaaggaagaggagaagcggctccggcgccacaaacttcagcctgctgaagcaggcaggcgatgtggaggagaatccaggacctatggtgctgcagacccaggtgtttatctccctgctgctgtggatctctggcgcctacggcgacatcgtgatgacccagagccctgattccctggccgtgtctctgggcgagagagccacaatcaactgtaagtcctctcagtccgtgctgtacagctccaacaataagaactacctggcctggtatcagcagaagcccggccagccacccaatctgctgatctattgggcatctaccaggcagagcggagtgcctgaccgcttctctggcagcggctccggcacagacttcaccctgacaatctctagcctgcaggccgaggacgtggccgtgtactattgccaccagtactacagcagccccctgaccttcggcggcggcacaaaggtggagatcaagaggaccgtggcagcaccaagcgtgttcatctttcctccaagcgacgagcagctgaagtccggcacagcctctgtggtgtgcctgctgaacaacttctaccctcgcgaggccaaggtgcagtggaaggtggataacgccctgcagtctggcaatagccaggagtccgtgaccgagcaggactctaaggatagcacatattccctgagctccaccctgacactgagcaaggccgattacgagaagcacaaggtgtatgcctgcgaggtcacccaccagggactgcggagccccgtcacaaagagcttcaacagaggagaatgctgataa

SEQ ID NO: 32 - protein, 739 aa, Artificial Sequence
  Synthetic Construct
MDWTWRILFLVAAATGTHAEVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVLTPDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSPDSLAVSLGERATINCKSSQSVLYSSNNKNYLAWYQQKPGQPPNLLIYWASTRQSGVPDRFSGSGSGTDFTLTISSLQAEDVAVYYCHQYYSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 33 - DNA, 1353 bp, Artificial Sequence
  Synthetic Construct
gaagtgcagctggtgcagtctggagccgaggtgaagaagcctggcgcctctgtgaaggtgagctgcaaggcctccggctacaccttcacaagctatgcaatccactgggtgaggcaggcaccaggacagagactggagtggatgggctggatcaaggccggcaacggcaatacaaggtacagccagaagtttcaggacagagtgaccatcacaagggacaccagcgcctccacagcctatatggagctgagctccctgcggtccgaggataccgccgtgtactattgcgccctgctgaccgtgctgacaccagacgatgccttcgacatctggggccagggcaccatggtgacagtgtctagcgccagcacaaagggaccatccgtgtttccactggcaccctcctctaagtctaccagcggcggcacagccgccctgggatgtctggtgaaggattacttccccgagcctgtgaccgtgtcttggaacagcggcgccctgaccagcggagtgcacacatttccagccgtgctgcagagctccggcctgtactccctgtctagcgtggtgaccgtgccctcctctagcctgggcacccagacatatatctgcaacgtgaatcacaagccatctaatacaaaggtggacaagaaggtggagcccaagagctgtgataagacccacacatgccctccctgtccagcaccagagctgctgggcggcccatccgtgttcctgtttccacccaagcctaaggacaccctgatgatctcccgcaccccagaggtgacatgcgtggtggtggacgtgtctcacgaggaccccgaggtgaagttcaactggtacgtggatggcgtggaggtgcacaatgccaagaccaagccacgggaggagcagtacaactctacctatcgcgtggtgagcgtgctgacagtgctgcaccaggactggctgaacggcaaggagtataagtgcaaggtgagcaataaggccctgcctgccccaatcgagaagaccatctccaaggccaagggccagcctagggagccacaggtgtacacactgcctccaagcagagacgagctgaccaagaaccaggtgtccctgacatgtctggtgaagggcttctatccttccgatatcgccgtggagtgggagtctaatggccagccagagaacaattacaagaccacaccccctgtgctggactccgatggctctttctttctgtatagcaagctgaccgtggacaagtccaggtggcagcagggcaacgtgttttcctgctctgtgatgcacgaggccctgcacaatcactacacccagaagagcctgtccctgtctccaggcaag

SEQ ID NO: 34 - protein, 451 aa, Artificial Sequence
  Synthetic Construct
EVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVLTPDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 666 bp, Artificial Sequence
  Synthetic Construct
gacatcgtgatgacccagagccctgattccctggccgtgtctctgggcgagagagccacaatcaactgtaagtcctctcagtccgtgctgtacagctccaacaataagaactacctggcctggtatcagcagaagcccggccagccacccaatctgctgatctattgggcatctaccaggcagagcggagtgcctgaccgcttctctggcagcggctccggcacagacttcaccctgacaatctctagcctgcaggccgaggacgtggccgtgtactattgccaccagtactacagcagccccctgaccttcggcggcggcacaaaggtggagatcaagaggaccgtggcagcaccaagcgtgttcatctttcctccaagcgacgagcagctgaagtccggcacagcctctgtggtgtgcctgctgaacaacttctaccctcgcgaggccaaggtgcagtggaaggtggataacgccctgcagtctggcaatagccaggagtccgtgaccgagcaggactctaaggatagcacatattccctgagctccaccctgacactgagcaaggccgattacgagaagcacaaggtgtatgcctgcgaggtcacccaccagggactgcggagccccgtcacaaagagcttcaacagaggagaatgctgataa

SEQ ID NO: 36 - protein, 220 aa, Artificial Sequence
  Synthetic Construct
DIVMTQSPDSLAVSLGERATINCKSSQSVLYSSNNKNYLAWYQQKPGQPPNLLIYWASTRQSGVPDRFSGSGSGTDFTLTISSLQAEDVAVYYCHQYYSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 37 - DNA, 363 bp, Artificial Sequence
  Synthetic Construct
gaagtgcagctggtgcagtctggagccgaggtgaagaagcctggcgcctctgtgaaggtgagctgcaaggcctccggctacaccttcacaagctatgcaatccactgggtgaggcaggcaccaggacagagactggagtggatgggctggatcaaggccggcaacggcaatacaaggtacagccagaagtttcaggacagagtgaccatcacaagggacaccagcgcctccacagcctatatggagctgagctccctgcggtccgaggataccgccgtgtactattgcgccctgctgaccgtgctgacaccagacgatgccttcgacatctggggccagggcaccatggtgacagtgtctagc

SEQ ID NO: 38 - protein, 121 aa, Artificial Sequence
  Synthetic Construct
EVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVLTPDDAFDIWGQGTMVTVSS

SEQ ID NO: 39 - DNA, 339 bp, Artificial Sequence
  Synthetic Construct
gacatcgtgatgacccagagccctgattccctggccgtgtctctgggcgagagagccacaatcaactgtaagtcctctcagtccgtgctgtacagctccaacaataagaactacctggcctggtatcagcagaagcccggccagccacccaatctgctgatctattgggcatctaccaggcagagcggagtgcctgaccgcttctctggcagcggctccggcacagacttcaccctgacaatctctagcctgcaggccgaggacgtggccgtgtactattgccaccagtactacagcagccccctgaccttcggcggcggcacaaaggtggagatcaag

SEQ ID NO: 40 - protein, 113 aa, Artificial Sequence
  Synthetic Construct
DIVMTQSPDSLAVSLGERATINCKSSQSVLYSSNNKNYLAWYQQKPGQPPNLLIYWASTRQSGVPDRFSGSGSGTDFTLTISSLQAEDVAVYYCHQYYSSPLTFGGGTKVEIK

SEQ ID NO: 41 - DNA, 327 bp, Artificial Sequence
  Synthetic Construct
aggaccgtggcagcaccaagcgtgttcatctttcctccaagcgacgagcagctgaagtccggcacagcctctgtggtgtgcctgctgaacaacttctaccctcgcgaggccaaggtgcagtggaaggtggataacgccctgcagtctggcaatagccaggagtccgtgaccgagcaggactctaaggatagcacatattccctgagctccaccctgacactgagcaaggccgattacgagaagcacaaggtgtatgcctgcgaggtcacccaccagggactgcggagccccgtcacaaagagcttcaacagaggagaatgctgataa

SEQ ID NO: 42 - protein, 107 aa, Artificial Sequence
  Synthetic Construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 43 - DNA, 2223 bp, Artificial Sequence
  Synthetic Construct
atggattggacatggagaatcctgttcctggtcgccgccgcaactgggactcacgcccaggtgcagctggtgcagtctggagccgaggtgaagaagcctggcgcctctgtgaaggtgagctgcaaggcctccggctacaccttcacaagctatgcaatccactgggtgaggcaggcaccaggacagagactggagtggatgggctggatcaaggccggcaacggcaatacaaggtacagccagaagtttcaggacagagtgaccatcacaagggacaccagcgcctccacagcctatatggagctgagctccctgcggtccgaggataccgccgtgtactattgcgccctgctgaccgtgctgacaccagacgatgccttcgacatctggggccagggcaccatggtgacagtgtctagcgccagcacaaagggaccatccgtgtttccactggcaccctcctctaagtctaccagcggcggcacagccgccctgggatgtctggtgaaggattacttccccgagcctgtgaccgtgtcttggaacagcggcgccctgaccagcggagtgcacacatttccagccgtgctgcagagctccggcctgtactccctgtctagcgtggtgaccgtgccctcctctagcctgggcacccagacatatatctgcaacgtgaatcacaagccatctaatacaaaggtggacaagaaggtggagcccaagagctgtgataagacccacacatgccctccctgtccagcaccagagctgctgggcggcccatccgtgttcctgtttccacccaagcctaaggacaccctgatgatctcccgcaccccagaggtgacatgcgtggtggtggacgtgtctcacgaggaccccgaggtgaagttcaactggtacgtggatggcgtggaggtgcacaatgccaagaccaagccacgggaggagcagtacaactctacctatcgcgtggtgagcgtgctgacagtgctgcaccaggactggctgaacggcaaggagtataagtgcaaggtgagcaataaggccctgcctgccccaatcgagaagaccatctccaaggccaagggccagcctagggagccacaggtgtacacactgcctccaagcagagacgagctgaccaagaaccaggtgtccctgacatgtctggtgaagggcttctatccttccgatatcgccgtggagtgggagtctaatggccagccagagaacaattacaagaccacaccccctgtgctggactccgatggctctttctttctgtatagcaagctgaccgtggacaagtccaggtggcagcagggcaacgtgttttcctgctctgtgatgcacgaggccctgcacaatcactacacccagaagagcctgtccctgtctccaggcaagaggggaaggaagaggagaagcggctccggcgccacaaacttcagcctgctgaagcaggcaggcgatgtggaggagaatccaggacctatggtgctgcagacccaggtgtttatctccctgctgctgtggatctctggcgcctacggcgacatcgtgatgacccagagccctgattccctggccgtgtctctgggcgagagagccacaatcaactgtaagtcctctcagtccgtgctgtacagctccaacaataagaactacctggcctggtatcagcagaagcccggccagccacccaagctgctgatctattgggcatctaccaggcagagcggagtgcctgaccgcttctctggcagcggctccggcacagacttcaccctgacaatctctagcctgcaggccgaggacgtggccgtgtactattgccaccagtactacagcagccccctgaccttcggcggcggcacaaaggtggagatcaagaggaccgtggcagcaccaagcgtgttcatctttcctccaagcgacgagcagctgaagtccggcacagcctctgtggtgtgcctgctgaacaacttctaccctcgcgaggccaaggtgcagtggaaggtggataacgccctgcagtctggcaatagccaggagtccgtgaccgagcaggactctaaggatagcacatattccctgagctccaccctgacactgagcaaggccgattacgagaagcacaaggtgtatgcctgcgaggtcacccaccagggactgcggagccccgtcacaaagagcttcaacagaggagaatgctgataa

SEQ ID NO: 44 - protein, 739 aa, Artificial Sequence
  Synthetic Construct
MDWTWRILFLVAAATGTHAQVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVLTPDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSPDSLAVSLGERATINCKSSQSVLYSSNNKNYLAWYQQKPGQPPKLLIYWASTRQSGVPDRFSGSGSGTDFTLTISSLQAEDVAVYYCHQYYSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 45 - DNA, 1353 bp, Artificial Sequence
  Synthetic Construct
caggtgcagctggtgcagtctggagccgaggtgaagaagcctggcgcctctgtgaaggtgagctgcaaggcctccggctacaccttcacaagctatgcaatccactgggtgaggcaggcaccaggacagagactggagtggatgggctggatcaaggccggcaacggcaatacaaggtacagccagaagtttcaggacagagtgaccatcacaagggacaccagcgcctccacagcctatatggagctgagctccctgcggtccgaggataccgccgtgtactattgcgccctgctgaccgtgctgacaccagacgatgccttcgacatctggggccagggcaccatggtgacagtgtctagcgccagcacaaagggaccatccgtgtttccactggcaccctcctctaagtctaccagcggcggcacagccgccctgggatgtctggtgaaggattacttccccgagcctgtgaccgtgtcttggaacagcggcgccctgaccagcggagtgcacacatttccagccgtgctgcagagctccggcctgtactccctgtctagcgtggtgaccgtgccctcctctagcctgggcacccagacatatatctgcaacgtgaatcacaagccatctaatacaaaggtggacaagaaggtggagcccaagagctgtgataagacccacacatgccctccctgtccagcaccagagctgctgggcggcccatccgtgttcctgtttccacccaagcctaaggacaccctgatgatctcccgcaccccagaggtgacatgcgtggtggtggacgtgtctcacgaggaccccgaggtgaagttcaactggtacgtggatggcgtggaggtgcacaatgccaagaccaagccacgggaggagcagtacaactctacctatcgcgtggtgagcgtgctgacagtgctgcaccaggactggctgaacggcaaggagtataagtgcaaggtgagcaataaggccctgcctgccccaatcgagaagaccatctccaaggccaagggccagcctagggagccacaggtgtacacactgcctccaagcagagacgagctgaccaagaaccaggtgtccctgacatgtctggtgaagggcttctatccttccgatatcgccgtggagtgggagtctaatggccagccagagaacaattacaagaccacaccccctgtgctggactccgatggctctttctttctgtatagcaagctgaccgtggacaagtccaggtggcagcagggcaacgtgttttcctgctctgtgatgcacgaggccctgcacaatcactacacccagaagagcctgtccctgtctccaggcaag

SEQ ID NO: 46 - protein, 451 aa, Artificial Sequence
  Synthetic Construct
QVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVLTPDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - DNA, 666 bp, Artificial Sequence
  Synthetic Construct
gacatcgtgatgacccagagccctgattccctggccgtgtctctgggcgagagagccacaatcaactgtaagtcctctcagtccgtgctgtacagctccaacaataagaactacctggcctggtatcagcagaagcccggccagccacccaagctgctgatctattgggcatctaccaggcagagcggagtgcctgaccgcttctctggcagcggctccggcacagacttcaccctgacaatctctagcctgcaggccgaggacgtggccgtgtactattgccaccagtactacagcagccccctgaccttcggcggcggcacaaaggtggagatcaagaggaccgtggcagcaccaagcgtgttcatctttcctccaagcgacgagcagctgaagtccggcacagcctctgtggtgtgcctgctgaacaacttctaccctcgcgaggccaaggtgcagtggaaggtggataacgccctgcagtctggcaatagccaggagtccgtgaccgagcaggactctaaggatagcacatattccctgagctccaccctgacactgagcaaggccgattacgagaagcacaaggtgtatgcctgcgaggtcacccaccagggactgcggagccccgtcacaaagagcttcaacagaggagaatgctgataa

SEQ ID NO: 48 - protein, 220 aa, Artificial Sequence
  Synthetic Construct
DIVMTQSPDSLAVSLGERATINCKSSQSVLYSSNNKNYLAWYQQKPGQPPKLLIYWASTRQSGVPDRFSGSGSGTDFTLTISSLQAEDVAVYYCHQYYSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 49 - DNA, 339 bp, Artificial Sequence
  Synthetic Construct
gacatcgtgatgacccagagccctgattccctggccgtgtctctgggcgagagagccacaatcaactgtaagtcctctcagtccgtgctgtacagctccaacaataagaactacctggcctggtatcagcagaagcccggccagccacccaagctgctgatctattgggcatctaccaggcagagcggagtgcctgaccgcttctctggcagcggctccggcacagacttcaccctgacaatctctagcctgcaggccgaggacgtggccgtgtactattgccaccagtactacagcagccccctgaccttcggcggcggcacaaaggtggagatcaag

SEQ ID NO: 50 - protein, 113 aa, Artificial Sequence
  Synthetic Construct
DIVMTQSPDSLAVSLGERATINCKSSQSVLYSSNNKNYLAWYQQKPGQPPKLLIYWASTRQSGVPDRFSGSGSGTDFTLTISSLQAEDVAVYYCHQYYSSPLTFGGGTKVEIK

SEQ ID NO: 51 - DNA, 2220 bp, Artificial Sequence
  Synthetic Construct
atggattggacatggaggattctgtttctggtcgccgccgctacaggaactcacgctcaggtgcagctggtgcagtcaggggccgaggtgaagaagcccggcgcctctgtgaaggtgagctgcaaggcctccggctacaccttcacaagctatgccatccactgggtgcggcaggcacctggacagcgcctggagtggatgggctggatcaaggccggcaacggcaataccaggtacagccagaagtttcaggacagggtgaccatcacaagagatacctccgcctctacagcctatatggagctgagctccctgagatccgaggacacagccgtgtactattgcgccctgctgaccgtgctgacacctgacgatgccttcgatatctggggccagggcaccatggtgacagtgtctagcgcctctaccaagggcccaagcgtgtttccactggcaccctcctctaagagcacctccggcggcacagccgccctgggctgtctggtgaaggactacttcccagagcccgtgaccgtgtcctggaactctggcgccctgaccagcggagtgcacacatttccagccgtgctgcagagctccggcctgtactccctgtctagcgtggtgaccgtgccctcctctagcctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacacactgatgatctctcggacccccgaggtgacatgcgtggtggtggacgtgagccacgaggaccccgaggtgaagttcaactggtacgtggatggcgtggaggtgcacaatgccaagaccaagcccagggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggcaaagggacagcctcgggagccacaggtgtacacactgcctccatcccgcgacgagctgaccaagaaccaggtgtctctgacatgtctggtgaagggcttctatccttctgatatcgccgtggagtgggagagcaatggccagccagagaacaattacaagaccacaccccctgtgctggacagcgatggctccttctttctgtatagcaagctgaccgtggataagtcccgctggcagcagggcaacgtgttcagctgtagcgtgatgcacgaagcactgcacaaccattacacccagaaaagcctgtcactgtcacccggcaaaaggggaaggaagaggagaagcggctccggcgccacaaacttcagcctgctgaagcaggcaggcgatgtggaggagaatccaggacctatggtgctgcagactcaggtgtttatttcactgctgctgtggatttcaggcgcttacggcgacattgtgatgacccagagccccgacagcctggccgtgtccctgggagagagggcaaccatcaactgcaagagctcccagtctgtgctgtactctagcaacaataagaattacctggcctggtatcagcagaagcccggccagccccctaagctgctgatctattgggcatctaccaggcagagcggagtgcctgacagattctccggctctggcagcggcacagacttcaccctgacaatctcctctctgcaggccgaggacgtggccgtgtactattgccaccagtactacagcagccccctgaccttcggcggcggcacaaaggtggagatcaagaggaccgtggcagcaccaagcgtgttcatctttccaccctccgacgagcagctgaagtccggcacagcctctgtggtgtgcctgctgaacaacttctacccccgcgaggccaaggtgcagtggaaggtggataacgccctgcagtccggcaattctcaggagagcgtgaccgagcaggacaaggattccacatattctctgtctagcaccctgacactgtccaaggccgattacgagaagcacaaggtgtatgcctgtgaggtcactcaccagggactgtcaagccccgtcacaaaatccttcaatagaggcgaatgctgataa

SEQ ID NO: 52 - protein, 738 aa, Artificial Sequence
  Synthetic Construct
MDWTWRILFLVAAATGTHAQVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVLTPDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSPDSLAVSLGERATINCKSSQSVLYSSNNKNYLAWYQQKPGQPPKLLIYWASTRQSGVPDRFSGSGSGTDFTLTISSLQAEDVAVYYCHQYYSSPLTFGGGTKVEIKRTVAAPSVFIFPPSDEQLKSGTASVVCLLNNFYPREAKVQWKVDNALQSGNSQESVTEQDKDSTYSLSSTLTLSKADYEKHKVYACEVTHQGLSSPVTKSFNRGEC

SEQ ID NO: 53 - DNA, 1359 bp, Artificial Sequence
  Synthetic Construct
caggtgcagctggtgcagtcaggggccgaggtgaagaagcccggcgcctctgtgaaggtgagctgcaaggcctccggctacaccttcacaagctatgccatccactgggtgcggcaggcacctggacagcgcctggagtggatgggctggatcaaggccggcaacggcaataccaggtacagccagaagtttcaggacagggtgaccatcacaagagatacctccgcctctacagcctatatggagctgagctccctgagatccgaggacacagccgtgtactattgcgccctgctgaccgtgctgacacctgacgatgccttcgatatctggggccagggcaccatggtgacagtgtctagcgcctctaccaagggcccaagcgtgtttccactggcaccctcctctaagagcacctccggcggcacagccgccctgggctgtctggtgaaggactacttcccagagcccgtgaccgtgtcctggaactctggcgccctgaccagcggagtgcacacatttccagccgtgctgcagagctccggcctgtactccctgtctagcgtggtgaccgtgccctcctctagcctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacacactgatgatctctcggacccccgaggtgacatgcgtggtggtggacgtgagccacgaggaccccgaggtgaagttcaactggtacgtggatggcgtggaggtgcacaatgccaagaccaagcccagggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggcaaagggacagcctcgggagccacaggtgtacacactgcctccatcccgcgacgagctgaccaagaaccaggtgtctctgacatgtctggtgaagggcttctatccttctgatatcgccgtggagtgggagagcaatggccagccagagaacaattacaagaccacaccccctgtgctggacagcgatggctccttctttctgtatagcaagctgaccgtggataagtcccgctggcagcagggcaacgtgttcagctgtagcgtgatgcacgaagcactgcacaaccattacacccagaaaagcctgtcactgtcacccggcaaatgataa

SEQ ID NO: 54 - protein, 451 aa, Artificial Sequence; identical to SEQ ID NO: 46
  Synthetic Construct

SEQ ID NO: 55 - DNA, 663 bp, Artificial Sequence
  Synthetic Construct
gacattgtgatgacccagagccccgacagcctggccgtgtccctgggagagagggcaaccatcaactgcaagagctcccagtctgtgctgtactctagcaacaataagaattacctggcctggtatcagcagaagcccggccagccccctaagctgctgatctattgggcatctaccaggcagagcggagtgcctgacagattctccggctctggcagcggcacagacttcaccctgacaatctcctctctgcaggccgaggacgtggccgtgtactattgccaccagtactacagcagccccctgaccttcggcggcggcacaaaggtggagatcaagaggaccgtggcagcaccaagcgtgttcatctttccaccctccgacgagcagctgaagtccggcacagcctctgtggtgtgcctgctgaacaacttctacccccgcgaggccaaggtgcagtggaaggtggataacgccctgcagtccggcaattctcaggagagcgtgaccgagcaggacaaggattccacatattctctgtctagcaccctgacactgtccaaggccgattacgagaagcacaaggtgtatgcctgtgaggtcactcaccagggactgtcaagccccgtcacaaaatccttcaatagaggcgaatgctgataa

SEQ ID NO: 56 - protein, 113 aa, Artificial Sequence; identical to SEQ ID NO: 50
  Synthetic Construct

SEQ ID NO: 57 - DNA, 363 bp, Artificial Sequence
  Synthetic Construct
caggtgcagctggtgcagtcaggggccgaggtgaagaagcccggcgcctctgtgaaggtgagctgcaaggcctccggctacaccttcacaagctatgccatccactgggtgcggcaggcacctggacagcgcctggagtggatgggctggatcaaggccggcaacggcaataccaggtacagccagaagtttcaggacagggtgaccatcacaagagatacctccgcctctacagcctatatggagctgagctccctgagatccgaggacacagccgtgtactattgcgccctgctgaccgtgctgacacctgacgatgccttcgatatctggggccagggcaccatggtgacagtgtctagc

SEQ ID NO: 58 - protein, 121 aa, Artificial Sequence
  Synthetic Construct
QVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVLTPDDAFDIWGQGTMVTVSS

SEQ ID NO: 59 - DNA, 324 bp, Artificial Sequence
  Synthetic Construct
aggaccgtggcagcaccaagcgtgttcatctttccaccctccgacgagcagctgaagtccggcacagcctctgtggtgtgcctgctgaacaacttctacccccgcgaggccaaggtgcagtggaaggtggataacgccctgcagtccggcaattctcaggagagcgtgaccgagcaggacaaggattccacatattctctgtctagcaccctgacactgtccaaggccgattacgagaagcacaaggtgtatgcctgtgaggtcactcaccagggactgtcaagccccgtcacaaaatccttcaatagaggcgaatgctgataa

SEQ ID NO: 60 - protein, 106 aa, Artificial Sequence
  Synthetic Construct
RTVAAPSVFIFPPSDEQLKSGTASVVCLLNNFYPREAKVQWKVDNALQSGNSQESVTEQDKDSTYSLSSTLTLSKADYEKHKVYACEVTHQGLSSPVTKSFNRGEC

SEQ ID NO: 61 - DNA, 2220 bp, Artificial Sequence
  Synthetic Construct
atggattggacatggaggattctgtttctggtcgccgccgctacaggaactcacgctcaggtgcagctggtgcagtcaggggccgaggtgaagaagcccggcgcctctgtgaaggtgagctgcaaggcctccggctacaccttcacaagctatgccatccactgggtgcggcaggcacctggacagcgcctggagtggatgggctggatcaaggccggcaacggcaataccaggtacagccagaagtttcaggacagggtgaccatcacaagagatacctccgcctctacagcctatatggagctgagctccctgagatccgaggacacagccgtgtactattgcgccctgctgaccgtgctgacacctgacgatgccttcgatatctggggccagggcaccatggtgacagtgtctagcgcctctaccaagggcccaagcgtgtttccactggcaccctcctctaagagcacctccggcggcacagccgccctgggctgtctggtgaaggactacttcccagagcccgtgaccgtgtcctggaactctggcgccctgaccagcggagtgcacacatttccagccgtgctgcagagctccggcctgtactccctgtctagcgtggtgaccgtgccctcctctagcctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacacactgatgatctctcggacccccgaggtgacatgcgtggtggtggacgtgagccacgaggaccccgaggtgaagttcaactggtacgtggatggcgtggaggtgcacaatgccaagaccaagcccagggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggcaaagggacagcctcgggagccacaggtgtacacactgcctccatcccgcgacgagctgaccaagaaccaggtgtctctgacatgtctggtgaagggcttctatccttctgatatcgccgtggagtgggagagcaatggccagccagagaacaattacaagaccacaccccctgtgctggacagcgatggctccttctttctgtatagcaagctgaccgtggataagtcccgctggcagcagggcaacgtgttcagctgtagcgtgatgcacgaagcactgcacaaccattacacccagaaaagcctgtcactgtcacccggcaaaaggggaaggaagaggagaagcggctccggcgccacaaacttcagcctgctgaagcaggcaggcgatgtggaggagaatccaggacctatggtgctgcagactcaggtgtttatttcactgctgctgtggatttcaggcgcttacggcgacattgtgatgacccagagccccgacagcctggccgtgtccctgggagagagggcaaccatcaactgcaagagctcccagtctgtgctgtactctagcaacaataagaattacctggcctggtatcagcagaagcccggccagccccctaagctgctgatctattgggcatctaccagggagagcggagtgcctgacagattctccggctctggcagcggcacagacttcaccctgacaatctcctctctgcaggccgaggacgtggccgtgtactattgccaccagtactacagcagccccctgaccttcggcggcggcacaaaggtggagatcaagaggaccgtggcagcaccaagcgtgttcatctttccaccctccgacgagcagctgaagtccggcacagcctctgtggtgtgcctgctgaacaacttctacccccgcgaggccaaggtgcagtggaaggtggataacgccctgcagtccggcaattctcaggagagcgtgaccgagcaggacaaggattccacatattctctgtctagcaccctgacactgtccaaggccgattacgagaagcacaaggtgtatgcctgtgaggtcactcaccagggactgtcaagccccgtcacaaaatccttcaatagaggcgaatgctgataa

SEQ ID NO: 62 - protein, 738 aa, Artificial Sequence
  Synthetic Construct
MDWTWRILFLVAAATGTHAQVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVLTPDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSPDSLAVSLGERATINCKSSQSVLYSSNNKNYLAWYQQKPGQPPKLLIYWASTRESGVPDRFSGSGSGTDFTLTISSLQAEDVAVYYCHQYYSSPLTFGGGTKVEIKRTVAAPSVFIFPPSDEQLKSGTASVVCLLNNFYPREAKVQWKVDNALQSGNSQESVTEQDKDSTYSLSSTLTLSKADYEKHKVYACEVTHQGLSSPVTKSFNRGEC

SEQ ID NO: 63 - DNA, 1359 bp, Artificial Sequence; identical to SEQ ID NO: 53
  Synthetic Construct

SEQ ID NO: 64 - protein, 451 aa, Artificial Sequence; identical to SEQ ID NO: 46
  Synthetic Construct

SEQ ID NO: 65 - DNA, 663 bp, Artificial Sequence
  Synthetic Construct
gacattgtgatgacccagagccccgacagcctggccgtgtccctgggagagagggcaaccatcaactgcaagagctcccagtctgtgctgtactctagcaacaataagaattacctggcctggtatcagcagaagcccggccagccccctaagctgctgatctattgggcatctaccagggagagcggagtgcctgacagattctccggctctggcagcggcacagacttcaccctgacaatctcctctctgcaggccgaggacgtggccgtgtactattgccaccagtactacagcagccccctgaccttcggcggcggcacaaaggtggagatcaagaggaccgtggcagcaccaagcgtgttcatctttccaccctccgacgagcagctgaagtccggcacagcctctgtggtgtgcctgctgaacaacttctacccccgcgaggccaaggtgcagtggaaggtggataacgccctgcagtccggcaattctcaggagagcgtgaccgagcaggacaaggattccacatattctctgtctagcaccctgacactgtccaaggccgattacgagaagcacaaggtgtatgcctgtgaggtcactcaccagggactgtcaagccccgtcacaaaatccttcaatagaggcgaatgctgataa

SEQ ID NO: 66 - protein, 219 aa, Artificial Sequence
  Synthetic Construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCHQYYSSPLTFGGGTKVEIKRTVAAPSVFIFPPSDEQLKSGTASVVCLLNNFYPREAKVQWKVDNALQSGNSQESVTEQDKDSTYSLSSTLTLSKADYEKHKVYACEVTHQGLSSPVTKSFNRGEC

SEQ ID NO: 67 - DNA, 339 bp, Artificial Sequence
  Synthetic Construct
gacattgtgatgacccagagccccgacagcctggccgtgtccctgggagagagggcaaccatcaactgcaagagctcccagtctgtgctgtactctagcaacaataagaattacctggcctggtatcagcagaagcccggccagccccctaagctgctgatctattgggcatctaccagggagagcggagtgcctgacagattctccggctctggcagcggcacagacttcaccctgacaatctcctctctgcaggccgaggacgtggccgtgtactattgccaccagtactacagcagccccctgaccttcggcggcggcacaaaggtggagatcaag

SEQ ID NO: 68 - protein, 113 aa, Artificial Sequence
  Synthetic Construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCHQYYSSPLTFGGGTKVEIK

SEQ ID NO: 69 - DNA, 2220 bp, Artificial Sequence
  Synthetic Construct
atggattggacatggaggattctgtttctggtcgccgccgctacaggaactcacgctcaggtgcagctggtgcagtcaggggccgaggtgaagaagcccggcgcctctgtgaaggtgagctgcaaggcctccggctacaccttcacaagctatgccatccactgggtgcggcaggcacctggacagcgcctggagtggatgggctggatcaaggccggcaacggcaataccaggtacagccagaagtttcagggcagggtgaccatcacaagagatacctccgcctctacagcctatatggagctgagctccctgagatccgaggacacagccgtgtactattgcgccctgctgaccgtgctgacacctgacgatgccttcgatatctggggccagggcaccatggtgacagtgtctagcgcctctaccaagggcccaagcgtgtttccactggcaccctcctctaagagcacctccggcggcacagccgccctgggctgtctggtgaaggactacttcccagagcccgtgaccgtgtcctggaactctggcgccctgaccagcggagtgcacacatttccagccgtgctgcagagctccggcctgtactccctgtctagcgtggtgaccgtgccctcctctagcctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacacactgatgatctctcggacccccgaggtgacatgcgtggtggtggacgtgagccacgaggaccccgaggtgaagttcaactggtacgtggatggcgtggaggtgcacaatgccaagaccaagcccagggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggcaaagggacagcctcgggagccacaggtgtacacactgcctccatcccgcgacgagctgaccaagaaccaggtgtctctgacatgtctggtgaagggcttctatccttctgatatcgccgtggagtgggagagcaatggccagccagagaacaattacaagaccacaccccctgtgctggacagcgatggctccttctttctgtatagcaagctgaccgtggataagtcccgctggcagcagggcaacgtgttcagctgtagcgtgatgcacgaagcactgcacaaccattacacccagaaaagcctgtcactgtcacccggcaaaaggggaaggaagaggagaagcggctccggcgccacaaacttcagcctgctgaagcaggcaggcgatgtggaggagaatccaggacctatggtgctgcagactcaggtgtttatttcactgctgctgtggatttcaggcgcttacggcgacattgtgatgacccagagccccgacagcctggccgtgtccctgggagagagggcaaccatcaactgcaagagctcccagtctgtgctgtactctagcaacaataagaattacctggcctggtatcagcagaagcccggccagccccctaagctgctgatctattgggcatctaccaggcagagcggagtgcctgacagattctccggctctggcagcggcacagacttcaccctgacaatctcctctctgcaggccgaggacgtggccgtgtactattgccaccagtactacagcagccccctgaccttcggcggcggcacaaaggtggagatcaagaggaccgtggcagcaccaagcgtgttcatctttccaccctccgacgagcagctgaagtccggcacagcctctgtggtgtgcctgctgaacaacttctacccccgcgaggccaaggtgcagtggaaggtggataacgccctgcagtccggcaattctcaggagagcgtgaccgagcaggacaaggattccacatattctctgtctagcaccctgacactgtccaaggccgattacgagaagcacaaggtgtatgcctgtgaggtcactcaccagggactgtcaagccccgtcacaaaatccttcaatagaggcgaatgctgataa

SEQ ID NO: 70 - protein, 738 aa, Artificial Sequence
  Synthetic Construct
MDWTWRILFLVAAATGTHAQVQLVQSGAEVKKPGASVKVSCKASGYTFTSYAIHWVRQAPGQRLEWMGWIKAGNGNTRYSQKFQGRVTITRDTSASTAYMELSSLRSEDTAVYYCALLTVLTPDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSPDSLAVSLGERATINCKSSQSVLYSSNNKNYLAWYQQKPGQPPKLLIYWASTRQSGVPDRFSGSGSGTDFTLTISSLQAEDVAVYYCHQYYSSPLTFGGGTKVEIKRTVAAPSVFIFPPSDEQLKSGTASVVCLLNNFYPREAKVQWKVDNALQSGNSQESVTEQDKDSTYSLSSTLTLSKADYEKHKVYACEVTHQGLSSPVTKSFNRGEC

SEQ ID NO: 71 - DNA, 1359 bp, Artificial Sequence
  Synthetic Construct
caggtgcagctggtgcagtcaggggccgaggtgaagaagcccggcgcctctgtgaaggtgagctgcaaggcctccggctacaccttcacaagctatgccatccactgggtgcggcaggcacctggacagcgcctggagtggatgggctggatcaaggccggcaacggcaataccaggtacagccagaagtttcagggcagggtgaccatcacaagagatacctccgcctctacagcctatatggagctgagctccctgagatccgaggacacagccgtgtactattgcgccctgctgaccgtgctgacacctgacgatgccttcgatatctggggccagggcaccatggtgacagtgtctagcgcctctaccaagggcccaagcgtgtttccactggcaccctcctctaagagcacctccggcggcacagccgccctgggctgtctggtgaaggactacttcccagagcccgtgaccgtgtcctggaactctggcgccctgaccagcggagtgcacacatttccagccgtgctgcagagctccggcctgtactccctgtctagcgtggtgaccgtgccctcctctagcctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacacactgatgatctctcggacccccgaggtgacatgcgtggtggtggacgtgagccacgaggaccccgaggtgaagttcaactggtacgtggatggcgtggaggtgcacaatgccaagaccaagcccagggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggcaaagggacagcctcgggagccacaggtgtacacactgcctccatcccgcgacgagctgaccaagaaccaggtgtctctgacatgtctggtgaagggcttctatccttctgatatcgccgtggagtgggagagcaatggccagccagagaacaattacaagaccacaccccctgtgctggacagcgatggctccttctttctgtatagcaagctgaccgtggataagtcccgctggcagcagggcaacgtgttcagctgtagcgtgatgcacgaagcactgcacaaccattacacccagaaaagcctgtcactgtcacccggcaaatgataa

SEQ ID NO: 72 - protein, 451 aa, Artificial Sequence
  Synthetic Construct
QVQLVQSGAEVKKPGASVKVSCKASGYTFTSYAIHWVRQAPGQRLEWMGWIKAGNGNTRYSQKFQGRVTITRDTSASTAYMELSSLRSEDTAVYYCALLTVLTPDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - DNA, 663 bp, Artificial Sequence; identical to SEQ ID NO: 55
  Synthetic Construct

SEQ ID NO: 74 - protein, 219 aa, Artificial Sequence
  Synthetic Construct
DIVMTQSPDSLAVSLGERATINCKSSQSVLYSSNNKNYLAWYQQKPGQPPKLLIYWASTRQSGVPDRFSGSGSGTDFTLTISSLQAEDVAVYYCHQYYSSPLTFGGGTKVEIKRTVAAPSVFIFPPSDEQLKSGTASVVCLLNNFYPREAKVQWKVDNALQSGNSQESVTEQDKDSTYSLSSTLTLSKADYEKHKVYACEVTHQGLSSPVTKSFNRGEC

SEQ ID NO: 75 - DNA, 363 bp, Artificial Sequence
  Synthetic Construct
caggtgcagctggtgcagtcaggggccgaggtgaagaagcccggcgcctctgtgaaggtgagctgcaaggcctccggctacaccttcacaagctatgccatccactgggtgcggcaggcacctggacagcgcctggagtggatgggctggatcaaggccggcaacggcaataccaggtacagccagaagtttcagggcagggtgaccatcacaagagatacctccgcctctacagcctatatggagctgagctccctgagatccgaggacacagccgtgtactattgcgccctgctgaccgtgctgacacctgacgatgccttcgatatctggggccagggcaccatggtgacagtgtctagc

SEQ ID NO: 76 - protein, 121 aa, Artificial Sequence
  Synthetic Construct
QVQLVQSGAEVKKPGASVKVSCKASGYTFTSYAIHWVRQAPGQRLEWMGWIKAGNGNTRYSQKFQGRVTITRDTSASTAYMELSSLRSEDTAVYYCALLTVLTPDDAFDIWGQGTMVTVSS

SEQ ID NO: 77 - DNA, 2220 bp, Artificial Sequence
  Synthetic Construct
atggattggacatggaggattctgtttctggtcgccgccgctacaggaactcacgctcaggtgcagctggtgcagtcaggggccgaggtgaagaagcccggcgcctctgtgaaggtgagctgcaaggcctccggctacaccttcacaagctatgccatccactgggtgcggcaggcacctggacagcgcctggagtggatgggctggatcaaggccggcaacggcaataccaggtacagccagaagtttcagggcagggtgaccatcacaagagatacctccgcctctacagcctatatggagctgagctccctgagatccgaggacacagccgtgtactattgcgccctgctgaccgtgctgacacctgacgatgccttcgatatctggggccagggcaccatggtgacagtgtctagcgcctctaccaagggcccaagcgtgtttccactggcaccctcctctaagagcacctccggcggcacagccgccctgggctgtctggtgaaggactacttcccagagcccgtgaccgtgtcctggaactctggcgccctgaccagcggagtgcacacatttccagccgtgctgcagagctccggcctgtactccctgtctagcgtggtgaccgtgccctcctctagcctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacacactgatgatctctcggacccccgaggtgacatgcgtggtggtggacgtgagccacgaggaccccgaggtgaagttcaactggtacgtggatggcgtggaggtgcacaatgccaagaccaagcccagggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggcaaagggacagcctcgggagccacaggtgtacacactgcctccatcccgcgacgagctgaccaagaaccaggtgtctctgacatgtctggtgaagggcttctatccttctgatatcgccgtggagtgggagagcaatggccagccagagaacaattacaagaccacaccccctgtgctggacagcgatggctccttctttctgtatagcaagctgaccgtggataagtcccgctggcagcagggcaacgtgttcagctgtagcgtgatgcacgaagcactgcacaaccattacacccagaaaagcctgtcactgtcacccggcaaaaggggaaggaagaggagaagcggctccggcgccacaaacttcagcctgctgaagcaggcaggcgatgtggaggagaatccaggacctatggtgctgcagactcaggtgtttatttcactgctgctgtggatttcaggcgcttacggcgacattgtgatgacccagagccccgacagcctggccgtgtccctgggagagagggcaaccatcaactgcaagagctcccagtctgtgctgtactctagcaacaataagaattacctggcctggtatcagcagaagcccggccagccccctaagctgctgatctattgggcatctaccagggagagcggagtgcctgacagattctccggctctggcagcggcacagacttcaccctgacaatctcctctctgcaggccgaggacgtggccgtgtactattgccaccagtactacagcagccccctgaccttcggcggcggcacaaaggtggagatcaagaggaccgtggcagcaccaagcgtgttcatctttccaccctccgacgagcagctgaagtccggcacagcctctgtggtgtgcctgctgaacaacttctacccccgcgaggccaaggtgcagtggaaggtggataacgccctgcagtccggcaattctcaggagagcgtgaccgagcaggacaaggattccacatattctctgtctagcaccctgacactgtccaaggccgattacgagaagcacaaggtgtatgcctgtgaggtcactcaccagggactgtcaagccccgtcacaaaatccttcaatagaggcgaatgctgataa

SEQ ID NO: 78 - protein, 738 aa, Artificial Sequence
  Synthetic Construct
MDWTWRILFLVAAATGTHAQVQLVQSGAEVKKPGASVKVSCKASGYTFTSYAIHWVRQAPGQRLEWMGWIKAGNGNTRYSQKFQGRVTITRDTSASTAYMELSSLRSEDTAVYYCALLTVLTPDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSPDSLAVSLGERATINCKSSQSVLYSSNNKNYLAWYQQKPGQPPKLLIYWASTRESGVPDRFSGSGSGTDFTLTISSLQAEDVAVYYCHQYYSSPLTFGGGTKVEIKRTVAAPSVFIFPPSDEQLKSGTASVVCLLNNFYPREAKVQWKVDNALQSGNSQESVTEQDKDSTYSLSSTLTLSKADYEKHKVYACEVTHQGLSSPVTKSFNRGEC

SEQ ID NO: 79 - DNA, 1359 bp, Artificial Sequence; identical to SEQ ID NO: 71
  Synthetic Construct

SEQ ID NO: 80 - protein, 451 aa, Artificial Sequence; identical to SEQ ID NO: 72
  Synthetic Construct

SEQ ID NO: 81 - DNA, 663 bp, Artificial Sequence; identical to SEQ ID NO: 65
  Synthetic Construct

SEQ ID NO: 82 - protein, 219 aa, Artificial Sequence; identical to SEQ ID NO: 66
  Synthetic Construct

SEQ ID NO: 83 - DNA, 21 bp, Artificial Sequence
  Synthetic Construct
aggggaaggaagaggagaagc

SEQ ID NO: 84 - protein, 7 aa, Artificial Sequence
  Synthetic Construct
RGRKRRS

SEQ ID NO: 85 - DNA, 66 bp, Artificial Sequence
  Synthetic Construct
ggctccggcgccacaaacttcagcctgctgaagcaggcaggcgatgtggaggagaatccaggacct

SEQ ID NO: 86 - protein, 22 aa, Artificial Sequence
  Synthetic Construct
GSGATNFSLLKQAGDVEENPGP

SEQ ID NO: 87 - DNA, 57 bp, Artificial Sequence
  Synthetic Construct
atggattggacatggaggattctgtttctggtcgccgccgctacaggaactcacgct

SEQ ID NO: 88 - protein, 19 aa, Artificial Sequence
  Synthetic Construct
MDWTWRILFLVAAATGTHA

SEQ ID NO: 89 - DNA, 60 bp, Artificial Sequence
  Synthetic Construct
atggtgctgcagactcaggtgtttatttcactgctgctgtggatttcaggcgcttacggc

SEQ ID NO: 90 - protein, 20 aa, Artificial Sequence
  Synthetic Construct
MVLQTQVFISLLLWISGAYG

SEQ ID NO: 91 - DNA, 60 bp, Artificial Sequence
  Synthetic Construct
atggcctggactcccctgctgctgcctctgctgactttttgcactgtctccgaagcctca

SEQ ID NO: 92 - protein, 20 aa, Artificial Sequence
  Synthetic Construct
MAWTPLLLPLLTFCTVSEAS

SEQ ID NO: 93 - DNA, 2199 bp, Artificial Sequence
  Synthetic Construct
atggactggacatggagaatcctgttcctggtcgcagccgctactgggactcacgcacagatgcagctggtcgaaagtgggggcggggtggtgcagcctggccggagcctgaggctgtcctgcaccgcatctggcttcaccttttccacatacgccatgcactgggtgcggcagtctccaggacagggcctgcagtgggtggccgtgatcagctatcactccacaaacaagtactatgaggactccgtgaggggcagattcaccatcagccgcgataactccaagaatacactgtacctgcagatgaatagcctgagggcagaggacaccgccgtgtactattgcgcccgcgacggctatagctcctctttctttgatttctggggccagggcaccctggtgacagtgagctccgcctctacaaagggaccaagcgtgtttccactggcaccctctagcaagtctaccagcggcggcacagccgccctgggatgtctggtgaaggattacttcccagagcccgtgaccgtgtcttggaacagcggcgccctgacctctggagtgcacacatttccagccgtgctgcagtcctctggcctgtacagcctgagctccgtggtgaccgtgccctctagctccctgggcacccagacatatatctgcaacgtgaatcacaagccatctaatacaaaggtggacaagaaggtggagcccaagagctgtgataagacccacacatgccctccctgtcctgcaccagagctgctgggcggcccatccgtgttcctgtttccacccaagcctaaggacaccctgatgatctccaggacccccgaggtgacatgcgtggtggtggacgtgtctcacgaggaccccgaggtgaagtttaactggtacgtggatggcgtggaggtgcacaatgccaagacaaagcctagggaggagcagtacaactccacctatagagtggtgtctgtgctgacagtgctgcaccaggactggctgaacggcaaggagtataagtgcaaggtgagcaataaggccctgcccgcccctatcgagaagaccatctccaaggccaagggccagcctagggagccacaggtgtacacactgcctccaagcagagacgagctgaccaagaaccaggtgtccctgacatgtctggtgaagggcttctatccttctgatatcgccgtggagtgggagagcaatggccagccagagaacaattacaagaccacaccccctgtgctggactccgatggctctttctttctgtatagcaagctgaccgtggataagtccagatggcagcagggcaacgtgttcagctgttctgtgatgcacgaagccctgcacaaccactacactcagaaaagcctgtcactgtcacctgggaaaaggggaaggaagaggagaagcggctccggcgccacaaacttcagcctgctgaagcaggcaggcgatgtggaggagaatccaggacctatggtgctgcagactcaggtgtttatttcactgctgctgtggatttcaggagcctacggggacattcagatgactcagagcccttctaccctgtccgcctctgtgggcgacagggtggccatcacatgcagagccagccagtccatctctcggtggctggcctggtaccagcagcagccaggcaaggcaccaaagctgctgatgagcggagcatccgtgctggagtctggagtgcctagccgcttcagcggctccggctctggaaccgagtttaccctgacaatcagctccctgcagccagacgatttcgccacatactattgccagcactacaactcttatttcgtgacctttggccagggcacaaaggtggagatcaagaggaccgtggccgcccccagcgtgttcatctttcccccttccgacgagcagctgaagtccggcacagcctctgtggtgtgcctgctgaacaacttctaccctcgggaggccaaggtgcagtggaaggtggataacgccctgcagtctggcaatagccaggagtccgtgaccgagcaggactctaaggatagcacatattccctgtctagcaccctgacactgtccaaggccgattacgagaagcacaaggtgtatgcctgtgaagtgacccaccagggactgcgaagccccgtgactaaatctttcaacagaggagaatgttgataa

SEQ ID NO: 94 - protein, 730 aa, Artificial Sequence
  Synthetic Construct
MDWTWRILFLVAAATGTHAQMQLVESGGGVVQPGRSLRLSCTASGFTFSTYAMHWVRQSPGQGLQWVAVISYHSTNKYYEDSVRGRFTISRDNSKNTLYLQMNSLRAEDTAVYYCARDGYSSSFFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSTLSASVGDRVAITCRASQSISRWLAWYQQQPGKAPKLLMSGASVLESGVPSRFSGSGSGTEFTLTISSLQPDDFATYYCQHYNSYFVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGE

SEQ ID NO: 95 - DNA, 1353 bp, Artificial Sequence
  Synthetic Construct
cagatgcagctggtcgaaagtgggggcggggtggtgcagcctggccggagcctgaggctgtcctgcaccgcatctggcttcaccttttccacatacgccatgcactgggtgcggcagtctccaggacagggcctgcagtgggtggccgtgatcagctatcactccacaaacaagtactatgaggactccgtgaggggcagattcaccatcagccgcgataactccaagaatacactgtacctgcagatgaatagcctgagggcagaggacaccgccgtgtactattgcgcccgcgacggctatagctcctctttctttgatttctggggccagggcaccctggtgacagtgagctccgcctctacaaagggaccaagcgtgtttccactggcaccctctagcaagtctaccagcggcggcacagccgccctgggatgtctggtgaaggattacttcccagagcccgtgaccgtgtcttggaacagcggcgccctgacctctggagtgcacacatttccagccgtgctgcagtcctctggcctgtacagcctgagctccgtggtgaccgtgccctctagctccctgggcacccagacatatatctgcaacgtgaatcacaagccatctaatacaaaggtggacaagaaggtggagcccaagagctgtgataagacccacacatgccctccctgtcctgcaccagagctgctgggcggcccatccgtgttcctgtttccacccaagcctaaggacaccctgatgatctccaggacccccgaggtgacatgcgtggtggtggacgtgtctcacgaggaccccgaggtgaagtttaactggtacgtggatggcgtggaggtgcacaatgccaagacaaagcctagggaggagcagtacaactccacctatagagtggtgtctgtgctgacagtgctgcaccaggactggctgaacggcaaggagtataagtgcaaggtgagcaataaggccctgcccgcccctatcgagaagaccatctccaaggccaagggccagcctagggagccacaggtgtacacactgcctccaagcagagacgagctgaccaagaaccaggtgtccctgacatgtctggtgaagggcttctatccttctgatatcgccgtggagtgggagagcaatggccagccagagaacaattacaagaccacaccccctgtgctggactccgatggctctttctttctgtatagcaagctgaccgtggataagtccagatggcagcagggcaacgtgttcagctgttctgtgatgcacgaagccctgcacaaccactacactcagaaaagcctgtcactgtcacctgggaaatgataa

SEQ ID NO: 96 - protein, 449 aa, Artificial Sequence
  Synthetic Construct
QMQLVESGGGVVQPGRSLRLSCTASGFTFSTYAMHWVRQSPGQGLQWVAVISYHSTNKYYEDSVRGRFTISRDNSKNTLYLQMNSLRAEDTAVYYCARDGYSSSFFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - DNA, 648 bp, Artificial Sequence
  Synthetic Construct
gacattcagatgactcagagcccttctaccctgtccgcctctgtgggcgacagggtggccatcacatgcagagccagccagtccatctctcggtggctggcctggtaccagcagcagccaggcaaggcaccaaagctgctgatgagcggagcatccgtgctggagtctggagtgcctagccgcttcagcggctccggctctggaaccgagtttaccctgacaatcagctccctgcagccagacgatttcgccacatactattgccagcactacaactcttatttcgtgacctttggccagggcacaaaggtggagatcaagaggaccgtggccgcccccagcgtgttcatctttcccccttccgacgagcagctgaagtccggcacagcctctgtggtgtgcctgctgaacaacttctaccctcgggaggccaaggtgcagtggaaggtggataacgccctgcagtctggcaatagccaggagtccgtgaccgagcaggactctaaggatagcacatattccctgtctagcaccctgacactgtccaaggccgattacgagaagcacaaggtgtatgcctgtgaagtgacccaccagggactgcgaagccccgtgactaaatctttcaacagaggagaatgttgataa

SEQ ID NO: 98 - protein, 214 aa, Artificial Sequence
  Synthetic Construct
DIQMTQSPSTLSASVGDRVAITCRASQSISRWLAWYQQQPGKAPKLLMSGASVLESGVPSRFSGSGSGTEFTLTISSLQPDDFATYYCQHYNSYFVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 99 - DNA, 377 bp, Artificial Sequence
  Synthetic Construct
cagatgcagctggtcgaaagtgggggcggggtggtgcagcctggccggagcctgaggctgtcctgcaccgcatctggcttcaccttttccacatacgccatgcactgggtgcggcagtctccaggacagggcctgcagtgggtggccgtgatcagctatcactccacaaacaagtactatgaggactccgtgaggggcagattcaccatcagccgcgataactccaagaatacactgtacctgcagatgaatagcctgagggcagaggacaccgccgtgtactattgcgcccgcgacggctatagctcctctttctttgatttctggggccagggcaccctggtgacagtgagctccgcctctacaaagggaccaag

SEQ ID NO: 100 - protein, 119 aa, Artificial Sequence
  Synthetic Construct
QMQLVESGGGVVQPGRSLRLSCTASGFTFSTYAMHWVRQSPGQGLQWVAVISYHSTNKYYEDSVRGRFTISRDNSKNTLYLQMNSLRAEDTAVYYCARDGYSSSFFDFWGQGTLVTVSS

SEQ ID NO: 101 - DNA, 321 bp, Artificial Sequence
  Synthetic Construct
gacattcagatgactcagagcccttctaccctgtccgcctctgtgggcgacagggtggccatcacatgcagagccagccagtccatctctcggtggctggcctggtaccagcagcagccaggcaaggcaccaaagctgctgatgagcggagcatccgtgctggagtctggagtgcctagccgcttcagcggctccggctctggaaccgagtttaccctgacaatcagctccctgcagccagacgatttcgccacatactattgccagcactacaactcttatttcgtgacctttggccagggcacaaaggtggagatcaag

SEQ ID NO: 102 - protein, 107 aa, Artificial Sequence
  Synthetic Construct
DIQMTQSPSTLSASVGDRVAITCRASQSISRWLAWYQQQPGKAPKLLMSGASVLESGVPSRFSGSGSGTEFTLTISSLQPDDFATYYCQHYNSYFVTFGQGTKVEIK

SEQ ID NO: 103 - DNA, 2199 bp, Artificial Sequence
  Synthetic Construct
atggactggacatggagaatcctgttcctggtcgcagccgctactgggactcacgcacagatgcagctggtcgaaagtgggggcggggtggtgcagcctggccggagcctgaggctgtcctgcaccgcatctggcttcaccttttccacatacgccatgcactgggtgcggcagtctccaggacagggcctgcagtgggtggccgtgatcagctatcactccacaaacaagtactatgaggactccgtgaggggcagattcaccatcagccgcgataactccaagaatacactgtacctgcagatgaatagcctgagggcagaggacaccgccgtgtactattgcgcccgcgacggctatagctcctctttctttgatttctggggccagggcaccctggtgacagtgagctccgcctctacaaagggaccaagcgtgtttccactggcaccctctagcaagtctaccagcggcggcacagccgccctgggatgtctggtgaaggattacttcccagagcccgtgaccgtgtcttggaacagcggcgccctgacctctggagtgcacacatttccagccgtgctgcagtcctctggcctgtacagcctgagctccgtggtgaccgtgccctctagctccctgggcacccagacatatatctgcaacgtgaatcacaagccatctaatacaaaggtggacaagaaggtggagcccaagagctgtgataagacccacacatgccctccctgtcctgcaccagagctgctgggcggcccatccgtgttcctgtttccacccaagcctaaggacaccctgatgatctccaggacccccgaggtgacatgcgtggtggtggacgtgtctcacgaggaccccgaggtgaagtttaactggtacgtggatggcgtggaggtgcacaatgccaagacaaagcctagggaggagcagtacaactccacctatagagtggtgtctgtgctgacagtgctgcaccaggactggctgaacggcaaggagtataagtgcaaggtgagcaataaggccctgcccgcccctatcgagaagaccatctccaaggccaagggccagcctagggagccacaggtgtacacactgcctccaagcagagacgagctgaccaagaaccaggtgtccctgacatgtctggtgaagggcttctatccttctgatatcgccgtggagtgggagagcaatggccagccagagaacaattacaagaccacaccccctgtgctggactccgatggctctttctttctgtatagcaagctgaccgtggataagtccagatggcagcagggcaacgtgttcagctgttctgtgatgcacgaagccctgcacaaccactacactcagaaaagcctgtcactgtcacctgggaaaaggggaaggaagaggagaagcggctccggcgccacaaacttcagcctgctgaagcaggcaggcgatgtggaggagaatccaggacctatggtgctgcagactcaggtgtttatttcactgctgctgtggatttcaggagcctacggggacattcagatgactcagagcccttctaccctgtccgcctctgtgggcgacagggtgaccatcacatgcagagccagccagtccatctctcggtggctggcctggtaccagcagaagccaggcaaggcaccaaagctgctgatgagcggagcatccgtgctggagtctggagtgcctagccgcttcagcggctccggctctggaaccgagtttaccctgacaatcagctccctgcagccagacgatttcgccacatactattgccagcactacaactcttatttcgtgacctttggccagggcacaaaggtggagatcaagaggaccgtggccgcccccagcgtgttcatctttcccccttccgacgagcagctgaagtccggcacagcctctgtggtgtgcctgctgaacaacttctaccctcgggaggccaaggtgcagtggaaggtggataacgccctgcagtctggcaatagccaggagtccgtgaccgagcaggactctaaggatagcacatattccctgtctagcaccctgacactgtccaaggccgattacgagaagcacaaggtgtatgcctgtgaagtgacccaccagggactgcgaagccccgtgactaaatctttcaacagaggagaatgttgataa

SEQ ID NO: 104 - protein, 731 aa, Artificial Sequence
  Synthetic Construct
MDWTWRILFLVAAATGTHAQMQLVESGGGVVQPGRSLRLSCTASGFTFSTYAMHWVRQSPGQGLQWVAVISYHSTNKYYEDSVRGRFTISRDNSKNTLYLQMNSLRAEDTAVYYCARDGYSSSFFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSTLSASVGDRVTITCRASQSISRWLAWYQQKPGKAPKLLMSGASVLESGVPSRFSGSGSGTEFTLTISSLQPDDFATYYCQHYNSYFVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 105 - DNA, 648 bp, Artificial Sequence
  Synthetic Construct
gacattcagatgactcagagcccttctaccctgtccgcctctgtgggcgacagggtgaccatcacatgcagagccagccagtccatctctcggtggctggcctggtaccagcagaagccaggcaaggcaccaaagctgctgatgagcggagcatccgtgctggagtctggagtgcctagccgcttcagcggctccggctctggaaccgagtttaccctgacaatcagctccctgcagccagacgatttcgccacatactattgccagcactacaactcttatttcgtgacctttggccagggcacaaaggtggagatcaagaggaccgtggccgcccccagcgtgttcatctttcccccttccgacgagcagctgaagtccggcacagcctctgtggtgtgcctgctgaacaacttctaccctcgggaggccaaggtgcagtggaaggtggataacgccctgcagtctggcaatagccaggagtccgtgaccgagcaggactctaaggatagcacatattccctgtctagcaccctgacactgtccaaggccgattacgagaagcacaaggtgtatgcctgtgaagtgacccaccagggactgcgaagccccgtgactaaatctttcaacagaggagaatgttgataa

SEQ ID NO: 106 - protein, 214 aa, Artificial Sequence
  Synthetic Construct
DIQMTQSPSTLSASVGDRVTITCRASQSISRWLAWYQQKPGKAPKLLMSGASVLESGVPSRFSGSGSGTEFTLTISSLQPDDFATYYCQHYNSYFVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 107 - DNA, 321 bp, Artificial Sequence
  Synthetic Construct
gacattcagatgactcagagcccttctaccctgtccgcctctgtgggcgacagggtgaccatcacatgcagagccagccagtccatctctcggtggctggcctggtaccagcagaagccaggcaaggcaccaaagctgctgatgagcggagcatccgtgctggagtctggagtgcctagccgcttcagcggctccggctctggaaccgagtttaccctgacaatcagctccctgcagccagacgatttcgccacatactattgccagcactacaactcttatttcgtgacctttggccagggcacaaaggtggagatcaag

SEQ ID NO: 108 - protein, 107 aa, Artificial Sequence
  Synthetic Construct
DIQMTQSPSTLSASVGDRVTITCRASQSISRWLAWYQQKPGKAPKLLMSGASVLESGVPSRFSGSGSGTEFTLTISSLQPDDFATYYCQHYNSYFVTFGQGTKVEIK

SEQ ID NO: 109 - DNA, 2199 bp, Artificial Sequence
  Synthetic Construct
atggactggacatggagaatcctgttcctggtcgcagccgctactgggactcacgcacaggtgcagctggtcgaaagtgggggcggggtggtgcagcctggccggagcctgaggctgtcctgcgccgcatctggcttcaccttttccacatacgccatgcactgggtgcggcaggccccaggaaagggcctggagtgggtggccgtgatcagctatcactccacaaacaagtactatgccgactccgtgaagggcagattcaccatcagccgcgataactccaagaatacactgtacctgcagatgaatagcctgagggcagaggacaccgccgtgtactattgcgcccgcgacggctatagctcctctttctttgatttctggggccagggcaccctggtgacagtgagctccgcctctacaaagggaccaagcgtgtttccactggcaccctctagcaagtctaccagcggcggcacagccgccctgggatgtctggtgaaggattacttcccagagcccgtgaccgtgtcttggaacagcggcgccctgacctctggagtgcacacatttccagccgtgctgcagtcctctggcctgtacagcctgagctccgtggtgaccgtgccctctagctccctgggcacccagacatatatctgcaacgtgaatcacaagccatctaatacaaaggtggacaagaaggtggagcccaagagctgtgataagacccacacatgccctccctgtcctgcaccagagctgctgggcggcccatccgtgttcctgtttccacccaagcctaaggacaccctgatgatctccaggacccccgaggtgacatgcgtggtggtggacgtgtctcacgaggaccccgaggtgaagtttaactggtacgtggatggcgtggaggtgcacaatgccaagacaaagcctagggaggagcagtacaactccacctatagagtggtgtctgtgctgacagtgctgcaccaggactggctgaacggcaaggagtataagtgcaaggtgagcaataaggccctgcccgcccctatcgagaagaccatctccaaggccaagggccagcctagggagccacaggtgtacacactgcctccaagcagagacgagctgaccaagaaccaggtgtccctgacatgtctggtgaagggcttctatccttctgatatcgccgtggagtgggagagcaatggccagccagagaacaattacaagaccacaccccctgtgctggactccgatggctctttctttctgtatagcaagctgaccgtggataagtccagatggcagcagggcaacgtgttcagctgttctgtgatgcacgaagccctgcacaaccactacactcagaaaagcctgtcactgtcacctgggaaaaggggaaggaagaggagaagcggctccggcgccacaaacttcagcctgctgaagcaggcaggcgatgtggaggagaatccaggacctatggtgctgcagactcaggtgtttatttcactgctgctgtggatttcaggagcctacggggacattcagatgactcagagcccttctaccctgtccgcctctgtgggcgacagggtggccatcacatgcagagccagccagtccatctctcggtggctggcctggtaccagcagcagccaggcaaggcaccaaagctgctgatgagcggagcatccgtgctggagtctggagtgcctagccgcttcagcggctccggctctggaaccgagtttaccctgacaatcagctccctgcagccagacgatttcgccacatactattgccagcactacaactcttatttcgtgacctttggccagggcacaaaggtggagatcaagaggaccgtggccgcccccagcgtgttcatctttcccccttccgacgagcagctgaagtccggcacagcctctgtggtgtgcctgctgaacaacttctaccctcgggaggccaaggtgcagtggaaggtggataacgccctgcagtctggcaatagccaggagtccgtgaccgagcaggactctaaggatagcacatattccctgtctagcaccctgacactgtccaaggccgattacgagaagcacaaggtgtatgcctgtgaagtgacccaccagggactgcgaagccccgtgactaaatctttcaacagaggagaatgttgataa

SEQ ID NO: 110 - protein, 731 aa, Artificial Sequence
  Synthetic Construct
MDWTWRILFLVAAATGTHAQVQLVESGGGVVQPGRSLRLSCAASGFTFSTYAMHWVRQAPGKGLEWVAVISYHSTNKYYADSVKGRFTISRDNSKNTLYLQMNSLRAEDTAVYYCARDGYSSSFFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSTLSASVGDRVAITCRASQSISRWLAWYQQQPGKAPKLLMSGASVLESGVPSRFSGSGSGTEFTLTISSLQPDDFATYYCQHYNSYFVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 111 - DNA, 1353 bp, Artificial Sequence
  Synthetic Construct
caggtgcagctggtcgaaagtgggggcggggtggtgcagcctggccggagcctgaggctgtcctgcgccgcatctggcttcaccttttccacatacgccatgcactgggtgcggcaggccccaggaaagggcctggagtgggtggccgtgatcagctatcactccacaaacaagtactatgccgactccgtgaagggcagattcaccatcagccgcgataactccaagaatacactgtacctgcagatgaatagcctgagggcagaggacaccgccgtgtactattgcgcccgcgacggctatagctcctctttctttgatttctggggccagggcaccctggtgacagtgagctccgcctctacaaagggaccaagcgtgtttccactggcaccctctagcaagtctaccagcggcggcacagccgccctgggatgtctggtgaaggattacttcccagagcccgtgaccgtgtcttggaacagcggcgccctgacctctggagtgcacacatttccagccgtgctgcagtcctctggcctgtacagcctgagctccgtggtgaccgtgccctctagctccctgggcacccagacatatatctgcaacgtgaatcacaagccatctaatacaaaggtggacaagaaggtggagcccaagagctgtgataagacccacacatgccctccctgtcctgcaccagagctgctgggcggcccatccgtgttcctgtttccacccaagcctaaggacaccctgatgatctccaggacccccgaggtgacatgcgtggtggtggacgtgtctcacgaggaccccgaggtgaagtttaactggtacgtggatggcgtggaggtgcacaatgccaagacaaagcctagggaggagcagtacaactccacctatagagtggtgtctgtgctgacagtgctgcaccaggactggctgaacggcaaggagtataagtgcaaggtgagcaataaggccctgcccgcccctatcgagaagaccatctccaaggccaagggccagcctagggagccacaggtgtacacactgcctccaagcagagacgagctgaccaagaaccaggtgtccctgacatgtctggtgaagggcttctatccttctgatatcgccgtggagtgggagagcaatggccagccagagaacaattacaagaccacaccccctgtgctggactccgatggctctttctttctgtatagcaagctgaccgtggataagtccagatggcagcagggcaacgtgttcagctgttctgtgatgcacgaagccctgcacaaccactacactcagaaaagcctgtcactgtcacctgggaaatgataa

SEQ ID NO: 112 - protein, 449 aa, Artificial Sequence
  Synthetic Construct
QVQLVESGGGVVQPGRSLRLSCAASGFTFSTYAMHWVRQAPGKGLEWVAVISYHSTNKYYADSVKGRFTISRDNSKNTLYLQMNSLRAEDTAVYYCARDGYSSSFFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - DNA, 377 bp, Artificial Sequence
  Synthetic Construct
caggtgcagctggtcgaaagtgggggcggggtggtgcagcctggccggagcctgaggctgtcctgcgccgcatctggcttcaccttttccacatacgccatgcactgggtgcggcaggccccaggaaagggcctggagtgggtggccgtgatcagctatcactccacaaacaagtactatgccgactccgtgaagggcagattcaccatcagccgcgataactccaagaatacactgtacctgcagatgaatagcctgagggcagaggacaccgccgtgtactattgcgcccgcgacggctatagctcctctttctttgatttctggggccagggcaccctggtgacagtgagctccgcctctacaaagggaccaag

SEQ ID NO: 114 - protein, 119 aa, Artificial Sequence
  Synthetic Construct
QVQLVESGGGVVQPGRSLRLSCAASGFTFSTYAMHWVRQAPGKGLEWVAVISYHSTNKYYADSVKGRFTISRDNSKNTLYLQMNSLRAEDTAVYYCARDGYSSSFFDFWGQGTLVTVSS

SEQ ID NO: 115 - DNA, 2199 bp, Artificial Sequence
  Synthetic Construct
atggactggacatggagaatcctgttcctggtcgcagccgctactgggactcacgcacaggtgcagctggtcgaaagtgggggcggggtggtgcagcctggccggagcctgaggctgtcctgcgccgcatctggcttcaccttttccacatacgccatgcactgggtgcggcaggccccaggaaagggcctggagtgggtggccgtgatcagctatcactccacaaacaagtactatgccgactccgtgaagggcagattcaccatcagccgcgataactccaagaatacactgtacctgcagatgaatagcctgagggcagaggacaccgccgtgtactattgcgcccgcgacggctatagctcctctttctttgatttctggggccagggcaccctggtgacagtgagctccgcctctacaaagggaccaagcgtgtttccactggcaccctctagcaagtctaccagcggcggcacagccgccctgggatgtctggtgaaggattacttcccagagcccgtgaccgtgtcttggaacagcggcgccctgacctctggagtgcacacatttccagccgtgctgcagtcctctggcctgtacagcctgagctccgtggtgaccgtgccctctagctccctgggcacccagacatatatctgcaacgtgaatcacaagccatctaatacaaaggtggacaagaaggtggagcccaagagctgtgataagacccacacatgccctccctgtcctgcaccagagctgctgggcggcccatccgtgttcctgtttccacccaagcctaaggacaccctgatgatctccaggacccccgaggtgacatgcgtggtggtggacgtgtctcacgaggaccccgaggtgaagtttaactggtacgtggatggcgtggaggtgcacaatgccaagacaaagcctagggaggagcagtacaactccacctatagagtggtgtctgtgctgacagtgctgcaccaggactggctgaacggcaaggagtataagtgcaaggtgagcaataaggccctgcccgcccctatcgagaagaccatctccaaggccaagggccagcctagggagccacaggtgtacacactgcctccaagcagagacgagctgaccaagaaccaggtgtccctgacatgtctggtgaagggcttctatccttctgatatcgccgtggagtgggagagcaatggccagccagagaacaattacaagaccacaccccctgtgctggactccgatggctctttctttctgtatagcaagctgaccgtggataagtccagatggcagcagggcaacgtgttcagctgttctgtgatgcacgaagccctgcacaaccactacactcagaaaagcctgtcactgtcacctgggaaaaggggaaggaagaggagaagcggctccggcgccacaaacttcagcctgctgaagcaggcaggcgatgtggaggagaatccaggacctatggtgctgcagactcaggtgtttatttcactgctgctgtggatttcaggagcctacggggacattcagatgactcagagcccttctaccctgtccgcctctgtgggcgacagggtgaccatcacatgcagagccagccagtccatctctcggtggctggcctggtaccagcagaagccaggcaaggcaccaaagctgctgatgagcggagcatccgtgctggagtctggagtgcctagccgcttcagcggctccggctctggaaccgagtttaccctgacaatcagctccctgcagccagacgatttcgccacatactattgccagcactacaactcttatttcgtgacctttggccagggcacaaaggtggagatcaagaggaccgtggccgcccccagcgtgttcatctttcccccttccgacgagcagctgaagtccggcacagcctctgtggtgtgcctgctgaacaacttctaccctcgggaggccaaggtgcagtggaaggtggataacgccctgcagtctggcaatagccaggagtccgtgaccgagcaggactctaaggatagcacatattccctgtctagcaccctgacactgtccaaggccgattacgagaagcacaaggtgtatgcctgtgaagtgacccaccagggactgcgaagccccgtgactaaatctttcaacagaggagaatgttgataa

SEQ ID NO: 116 - protein, 731 aa, Artificial Sequence
  Synthetic Construct
MDWTWRILFLVAAATGTHAQVQLVESGGGVVQPGRSLRLSCAASGFTFSTYAMHWVRQAPGKGLEWVAVISYHSTNKYYADSVKGRFTISRDNSKNTLYLQMNSLRAEDTAVYYCARDGYSSSFFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSTLSASVGDRVTITCRASQSISRWLAWYQQKPGKAPKLLMSGASVLESGVPSRFSGSGSGTEFTLTISSLQPDDFATYYCQHYNSYFVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 117 - DNA, 2205 bp, Artificial Sequence
  Synthetic Construct
atggattggacatggaggattctgtttctggtcgccgccgcaactggaactcacgcacaggtgcagctggtgcagtcaggagccgaggtgaagaagcccggcgcctctgtgaaggtgagctgcaaggcctccggctacaccttcacaagctatgccatccactgggtgcggcaggcacctggacagcgcctggagtggatgggctggatcaaggccggcaacggcaataccaggtacagccagaagtttcaggacagggtgaccatcacaagagatacctccgcctctacagcctatatggagctgagctccctgagatccgaggacacagccgtgtactattgcgccctgctgaccgtgctgacacctgacgatgccttcgatatctggggccagggcaccatggtgacagtgtctagcgcctctaccaagggcccaagcgtgtttccactggcaccctcctctaagagcacctccggcggcacagccgccctgggctgtctggtgaaggactacttcccagagcccgtgaccgtgtcctggaactctggcgccctgaccagcggagtgcacacatttccagccgtgctgcagagctccggcctgtactccctgtctagcgtggtgaccgtgccctcctctagcctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacacactgatgatctctcggacccccgaggtgacatgcgtggtggtggacgtgagccacgaggaccccgaggtgaagttcaactggtacgtggatggcgtggaggtgcacaatgccaagaccaagcccagggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggcaaagggacagcctcgggagccacaggtgtacacactgcctccatcccgcgacgagctgaccaagaaccaggtgtctctgacatgtctggtgaagggcttctatccttctgatatcgccgtggagtgggagagcaatggccagccagagaacaattacaagaccacaccccctgtgctggacagcgatggctccttctttctgtatagcaagctgaccgtggataagtcccgctggcagcagggcaacgtgttcagctgtagcgtgatgcacggagccctgcacaaccactacactcagaaatccctgtcactgtcccctggcaaaaggggaaggaagaggagaagcggctccggcgccacaaacttcagcctgctgaagcaggcaggcgatgtggaggagaatccaggacctatggtgctgcagactcaggtgtttatttcactgctgctgtggatttcaggagcctacggggacattcagatgactcagagcccttctaccctgtccgcctctgtgggcgacagggtggccatcacatgcagagccagccagtccatctctcggtggctggcctggtaccagcagcagccaggcaaggcaccaaagctgctgatgagcggagcatccgtgctggagtctggagtgcctagccgcttcagcggctccggctctggaaccgagtttaccctgacaatcagctccctgcagccagacgatttcgccacatactattgccagcactacaactcttatttcgtgacctttggccagggcacaaaggtggagatcaagaggaccgtggccgcccccagcgtgttcatctttcccccttccgacgagcagctgaagtccggcacagcctctgtggtgtgcctgctgaacaacttctaccctcgggaggccaaggtgcagtggaaggtggataacgccctgcagtctggcaatagccaggagtccgtgaccgagcaggactctaaggatagcacatattccctgtctagcaccctgacactgtccaaggccgattacgagaagcacaaggtgtatgcctgtgaagtgacccaccagggactgcgaagccccgtgactaaatctttcaacagaggagaatgttgataa

SEQ ID NO: 118 - protein, 733 aa, Artificial Sequence
  Synthetic Construct
MDWTWRILFLVAAATGTHAQVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVLTPDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGKRGRKRRSGSGATNFSLLKQAGDVEENPGPMVLQTQVFISLLLWISGAYGDIQMTQSPSTLSASVGDRVAITCRASQSISRWLAWYQQQPGKAPKLLMSGASVLESGVPSRFSGSGSGTEFTLTISSLQPDDFATYYCQHYNSYFVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 119 - DNA, 1359 bp, Artificial Sequence
  Synthetic Construct
caggtgcagctggtgcagtcaggagccgaggtgaagaagcccggcgcctctgtgaaggtgagctgcaaggcctccggctacaccttcacaagctatgccatccactgggtgcggcaggcacctggacagcgcctggagtggatgggctggatcaaggccggcaacggcaataccaggtacagccagaagtttcaggacagggtgaccatcacaagagatacctccgcctctacagcctatatggagctgagctccctgagatccgaggacacagccgtgtactattgcgccctgctgaccgtgctgacacctgacgatgccttcgatatctggggccagggcaccatggtgacagtgtctagcgcctctaccaagggcccaagcgtgtttccactggcaccctcctctaagagcacctccggcggcacagccgccctgggctgtctggtgaaggactacttcccagagcccgtgaccgtgtcctggaactctggcgccctgaccagcggagtgcacacatttccagccgtgctgcagagctccggcctgtactccctgtctagcgtggtgaccgtgccctcctctagcctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacacactgatgatctctcggacccccgaggtgacatgcgtggtggtggacgtgagccacgaggaccccgaggtgaagttcaactggtacgtggatggcgtggaggtgcacaatgccaagaccaagcccagggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggcaaagggacagcctcgggagccacaggtgtacacactgcctccatcccgcgacgagctgaccaagaaccaggtgtctctgacatgtctggtgaagggcttctatccttctgatatcgccgtggagtgggagagcaatggccagccagagaacaattacaagaccacaccccctgtgctggacagcgatggctccttctttctgtatagcaagctgaccgtggataagtcccgctggcagcagggcaacgtgttcagctgtagcgtgatgcacggagccctgcacaaccactacactcagaaatccctgtcactgtcccctggcaaatgataa

SEQ ID NO: 120 - protein, 451 aa, Artificial Sequence
  Synthetic Construct
QVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVLTPDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 121 - DNA, 363 bp, Artificial Sequence
  Synthetic Construct
caggtgcagctggtgcagtcaggagccgaggtgaagaagcccggcgcctctgtgaaggtgagctgcaaggcctccggctacaccttcacaagctatgccatccactgggtgcggcaggcacctggacagcgcctggagtggatgggctggatcaaggccggcaacggcaataccaggtacagccagaagtttcaggacagggtgaccatcacaagagatacctccgcctctacagcctatatggagctgagctccctgagatccgaggacacagccgtgtactattgcgccctgctgaccgtgctgacacctgacgatgccttcgatatctggggccagggcaccatggtgacagtgtctagc

SEQ ID NO: 122 - protein, 121 aa, Artificial Sequence; identical to SEQ ID NO: 58
  Synthetic Construct

SEQ ID NO: 123 - DNA, 996 bp, Artificial Sequence
  Synthetic Construct
gcctctaccaagggcccaagcgtgtttccactggcaccctcctctaagagcacctccggcggcacagccgccctgggctgtctggtgaaggactacttcccagagcccgtgaccgtgtcctggaactctggcgccctgaccagcggagtgcacacatttccagccgtgctgcagagctccggcctgtactccctgtctagcgtggtgaccgtgccctcctctagcctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacacactgatgatctctcggacccccgaggtgacatgcgtggtggtggacgtgagccacgaggaccccgaggtgaagttcaactggtacgtggatggcgtggaggtgcacaatgccaagaccaagcccagggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggcaaagggacagcctcgggagccacaggtgtacacactgcctccatcccgcgacgagctgaccaagaaccaggtgtctctgacatgtctggtgaagggcttctatccttctgatatcgccgtggagtgggagagcaatggccagccagagaacaattacaagaccacaccccctgtgctggacagcgatggctccttctttctgtatagcaagctgaccgtggataagtcccgctggcagcagggcaacgtgttcagctgtagcgtgatgcacggagccctgcacaaccactacactcagaaatccctgtcactgtcccctggcaaatgataa

SEQ ID NO: 124 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 125 - DNA, 2205 bp, Artificial Sequence
  Synthetic Construct
atggattggacatggaggattctgtttctggtcgccgccgcaactggaactcacgcacaggtgcagctggtgcagtcaggagccgaggtgaagaagcccggcgcctctgtgaaggtgagctgcaaggcctccggctacaccttcacaagctatgccatccactgggtgcggcaggcacctggacagcgcctggagtggatgggctggatcaaggccggcaacggcaataccaggtacagccagaagtttcaggacagggtgaccatcacaagagatacctccgcctctacagcctatatggagctgagctccctgagatccgaggacacagccgtgtactattgcgccctgctgaccgtgctgacacctgacgatgccttcgatatctggggccagggcaccatggtgacagtgtctagcgcctctaccaagggcccaagcgtgtttccactggcaccctcctctaagagcacctccggcggcacagccgccctgggctgtctggtgaaggactacttcccagagcccgtgaccgtgtcctggaactctggcgccctgaccagcggagtgcacacatttccagccgtgctgcagagctccggcctgtactccctgtctagcgtggtgaccgtgccctcctctagcctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacacactgatgatctctcggacccccgaggtgacatgcgtggtggtggacgtgagccacgaggaccccgaggtgaagttcaactggtacgtggatggcgtggaggtgcacaatgccaagaccaagcccagggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggcaaagggacagcctcgggagccacaggtgtacacactgcctccatcccgcgacgagctgaccaagaaccaggtgtctctgacatgtctggtgaagggcttctatccttctgatatcgccgtggagtgggagagcaatggccagccagagaacaattacaagaccacaccccctgtgctggacagcgatggctccttctttctgtatagcaagctgaccgtggataagtcccgctggcagcagggcaacgtgttcagctgtagcgtgatgcacggagccctgcacaaccactacactcagaaatccctgtcactgtcccctggcaaaaggggaaggaagaggagaagcggctccggcgccacaaacttcagcctgctgaagcaggcaggcgatgtggaggagaatccaggacctatggtgctgcagactcaggtgtttatttcactgctgctgtggatttcaggagcctacggggacattcagatgactcagagcccttctaccctgtccgcctctgtgggcgacagggtgaccatcacatgcagagccagccagtccatctctcggtggctggcctggtaccagcagaagccaggcaaggcaccaaagctgctgatgagcggagcatccgtgctggagtctggagtgcctagccgcttcagcggctccggctctggaaccgagtttaccctgacaatcagctccctgcagccagacgatttcgccacatactattgccagcactacaactcttatttcgtgacctttggccagggcacaaaggtggagatcaagaggaccgtggccgcccccagcgtgttcatctttcccccttccgacgagcagctgaagtccggcacagcctctgtggtgtgcctgctgaacaacttctaccctcgggaggccaaggtgcagtggaaggtggataacgccctgcagtctggcaatagccaggagtccgtgaccgagcaggactctaaggatagcacatattccctgtctagcaccctgacactgtccaaggccgattacgagaagcacaaggtgtatgcctgtgaagtgacccaccagggactgcgaagccccgtgactaaatctttcaacagaggagaatgttgataa

SEQ ID NO: 126 - protein, 733 aa, Artificial Sequence
  Synthetic Construct
MDWTWRILFLVAAATGTHAQVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVLTPDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGKRGRKRRSGSGATNFSLLKQAGDVEENPGPMVLQTQVFISLLLWISGAYGDIQMTQSPSTLSASVGDRVTITCRASQSISRWLAWYQQKPGKAPKLLMSGASVLESGVPSRFSGSGSGTEFTLTISSLQPDDFATYYCQHYNSYFVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 127 - DNA, 2205 bp, Artificial Sequence; identical to SEQ ID NO: 117
  Synthetic Construct

SEQ ID NO: 128 - protein, 733 aa, Artificial Sequence
  Synthetic Construct
MDWTWRILFLVAAATGTHAQVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVLTPDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEAPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSTLSASVGDRVAITCRASQSISRWLAWYQQQPGKAPKLLMSGASVLESGVPSRFSGSGSGTEFTLTISSLQPDDFATYYCQHYNSYFVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 129 - DNA, 1359 bp, Artificial Sequence
  Synthetic Construct
caggtgcagctggtgcagtcaggagccgaggtgaagaagcccggcgcctctgtgaaggtgagctgcaaggcctccggctacaccttcacaagctatgccatccactgggtgcggcaggcacctggacagcgcctggagtggatgggctggatcaaggccggcaacggcaataccaggtacagccagaagtttcaggacagggtgaccatcacaagagatacctccgcctctacagcctatatggagctgagctccctgagatccgaggacacagccgtgtactattgcgccctgctgaccgtgctgacacctgacgatgccttcgatatctggggccagggcaccatggtgacagtgtctagcgcctctaccaagggcccaagcgtgtttccactggcaccctcctctaagagcacctccggcggcacagccgccctgggctgtctggtgaaggactacttcccagagcccgtgaccgtgtcctggaactctggcgccctgaccagcggagtgcacacatttccagccgtgctgcagagctccggcctgtactccctgtctagcgtggtgaccgtgccctcctctagcctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacacactgatgatctctcggacccccgaggtgacatgcgtggtggtggacgtgagccacgaggcccccgaggtgaagttcaactggtacgtggatggcgtggaggtgcacaatgccaagaccaagcccagggaggagcagtacaactccacctatagagtggtgtctgtgctgacagtgctgcaccaggactggctgaacggcaaggagtataagtgcgccgtgtccaataaggccctgcccgcccctatcgagaagaccatctctaaggcaaagggacagcctcgggagccacaggtgtacacactgcctccatcccgcgacgagctgaccaagaaccaggtgtctctgacatgtctggtgaagggcttctatccttctgatatcgccgtggagtgggagagcaatggccagccagagaacaattacaagaccacaccccctgtgctggacagcgatggctccttctttctgtatagcaagctgaccgtggataagtcccgctggcagcagggcaacgtgttcagctgtagcgtgatgcacgaggccctgcacaaccactacactcagaaatccctgtcactgtcccctggcaaatgataa

SEQ ID NO: 130 - protein, 451 aa, Artificial Sequence
  Synthetic Construct
QVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVLTPDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEAPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - DNA, 363 bp, Artificial Sequence; identical to SEQ ID NO: 121
  Synthetic Construct

SEQ ID NO: 132 - protein, 121 aa, Artificial Sequence; identical to SEQ ID NO: 58
  Synthetic Construct

SEQ ID NO: 133 - DNA, 996 bp, Artificial Sequence
  Synthetic Construct
gcctctaccaagggcccaagcgtgtttccactggcaccctcctctaagagcacctccggcggcacagccgccctgggctgtctggtgaaggactacttcccagagcccgtgaccgtgtcctggaactctggcgccctgaccagcggagtgcacacatttccagccgtgctgcagagctccggcctgtactccctgtctagcgtggtgaccgtgccctcctctagcctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacacactgatgatctctcggacccccgaggtgacatgcgtggtggtggacgtgagccacgaggcccccgaggtgaagttcaactggtacgtggatggcgtggaggtgcacaatgccaagaccaagcccagggaggagcagtacaactccacctatagagtggtgtctgtgctgacagtgctgcaccaggactggctgaacggcaaggagtataagtgcgccgtgtccaataaggccctgcccgcccctatcgagaagaccatctctaaggcaaagggacagcctcgggagccacaggtgtacacactgcctccatcccgcgacgagctgaccaagaaccaggtgtctctgacatgtctggtgaagggcttctatccttctgatatcgccgtggagtgggagagcaatggccagccagagaacaattacaagaccacaccccctgtgctggacagcgatggctccttctttctgtatagcaagctgaccgtggataagtcccgctggcagcagggcaacgtgttcagctgtagcgtgatgcacgaggccctgcacaaccactacactcagaaatccctgtcactgtcccctggcaaatgataa

SEQ ID NO: 134 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEAPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - DNA, 2205 bp, Artificial Sequence
  Synthetic Construct
atggattggacatggaggattctgtttctggtcgccgccgcaactggaactcacgcacaggtgcagctggtgcagtcaggagccgaggtgaagaagcccggcgcctctgtgaaggtgagctgcaaggcctccggctacaccttcacaagctatgccatccactgggtgcggcaggcacctggacagcgcctggagtggatgggctggatcaaggccggcaacggcaataccaggtacagccagaagtttcaggacagggtgaccatcacaagagatacctccgcctctacagcctatatggagctgagctccctgagatccgaggacacagccgtgtactattgcgccctgctgaccgtgctgacacctgacgatgccttcgatatctggggccagggcaccatggtgacagtgtctagcgcctctaccaagggcccaagcgtgtttccactggcaccctcctctaagagcacctccggcggcacagccgccctgggctgtctggtgaaggactacttcccagagcccgtgaccgtgtcctggaactctggcgccctgaccagcggagtgcacacatttccagccgtgctgcagagctccggcctgtactccctgtctagcgtggtgaccgtgccctcctctagcctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacacactgatgatctctcggacccccgaggtgacatgcgtggtggtggacgtgagccacgaggcccccgaggtgaagttcaactggtacgtggatggcgtggaggtgcacaatgccaagaccaagcccagggaggagcagtacaactccacctatagagtggtgtctgtgctgacagtgctgcaccaggactggctgaacggcaaggagtataagtgcgccgtgtccaataaggccctgcccgcccctatcgagaagaccatctctaaggcaaagggacagcctcgggagccacaggtgtacacactgcctccatcccgcgacgagctgaccaagaaccaggtgtctctgacatgtctggtgaagggcttctatccttctgatatcgccgtggagtgggagagcaatggccagccagagaacaattacaagaccacaccccctgtgctggacagcgatggctccttctttctgtatagcaagctgaccgtggataagtcccgctggcagcagggcaacgtgttcagctgtagcgtgatgcacgaggccctgcacaaccactacactcagaaatccctgtcactgtcccctggcaaaaggggaaggaagaggagaagcggctccggcgccacaaacttcagcctgctgaagcaggcaggcgatgtggaggagaatccaggacctatggtgctgcagactcaggtgtttatttcactgctgctgtggatttcaggagcctacggggacattcagatgactcagagcccttctaccctgtccgcctctgtgggcgacagggtgaccatcacatgcagagccagccagtccatctctcggtggctggcctggtaccagcagaagccaggcaaggcaccaaagctgctgatgagcggagcatccgtgctggagtctggagtgcctagccgcttcagcggctccggctctggaaccgagtttaccctgacaatcagctccctgcagccagacgatttcgccacatactattgccagcactacaactcttatttcgtgacctttggccagggcacaaaggtggagatcaagaggaccgtggccgcccccagcgtgttcatctttcccccttccgacgagcagctgaagtccggcacagcctctgtggtgtgcctgctgaacaacttctaccctcgggaggccaaggtgcagtggaaggtggataacgccctgcagtctggcaatagccaggagtccgtgaccgagcaggactctaaggatagcacatattccctgtctagcaccctgacactgtccaaggccgattacgagaagcacaaggtgtatgcctgtgaagtgacccaccagggactgcgaagccccgtgactaaatctttcaacagaggagaatgttgataa

SEQ ID NO: 136 - protein, 733 aa, Artificial Sequence
  Synthetic Construct
MDWTWRILFLVAAATGTHAQVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVLTPDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEAPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSTLSASVGDRVTITCRASQSISRWLAWYQQKPGKAPKLLMSGASVLESGVPSRFSGSGSGTEFTLTISSLQPDDFATYYCQHYNSYFVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 137 - DNA, 2199 bp, Artificial Sequence
  Synthetic Construct
atggactggacctggagaatcctgttcctggtggcagcagcaaccggaacacacgcccaggtgaagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacagtgtttatgcagatggactctctgcgggccgaggataccgccgtgtactattgccaccgcaatttctacgacggcagcggcccttttgattattggggccagggcaccctggtgacagtgagctccgccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggaccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacgaggccctgcacaatcactacacacagaagagcctgtccctgtctcctggcaagaggggaaggaagaggagaagcggctccggcgccacaaacttcagcctgctgaagcaggcaggcgatgtggaggagaatccaggacctatggtgctgcagacccaggtgttcatcagcctgctgctgtggatctccggcgcctacggcgacatccagatgacccagtctccaagcacactgtccgcctctgtgggcgatagggtgaccatcacatgcagagccagccagtttatctcccggtggctggcctggtaccagcagaagccaggcaaggcccccaagctgctgatctataaggccagctccctggagtctggagtgcctagccgcttcagcggctccggctctgagacacactttaccctgacaatctctagcctgcagccagacgatgtggccacctactattgccaggagtacacctcctatggcaggacattcggccagggcaccaaggtggagatcaagacagtggccgcccccagcgtgttcatctttccccctagcgacgagcagctgaagtccggcaccgcctctgtggtgtgcctgctgaacaacttctaccctcgggaggccaaggtgcagtggaaggtggataacgccctgcagagcggcaattcccaggagtctgtgacagagcaggacagcaaggattccacctattctctgtcctctaccctgacactgtccaaggccgactacgagaagcacaaggtgtatgcctgcgaggtgacacaccagggcctgagctcccccgtgaccaagagcttcaacaggggcgagtgttgataa

SEQ ID NO: 138 - protein, 731 aa, Artificial Sequence
  Synthetic Construct
MDWTWRILFLVAAATGTHAQVKLVESGGGVVQPGRSLRLSCEASGFIFSTYGMHWVRQAPGKGLEWVAVIWFDGSNIYYADSVKGRFTISRDNSKNTVFMQMDSLRAEDTAVYYCHRNFYDGSGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSTLSASVGDRVTITCRASQFISRWLAWYQQKPGKAPKLLIYKASSLESGVPSRFSGSGSETHFTLTISSLQPDDVATYYCQEYTSYGRTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - DNA, 1356 bp, Artificial Sequence
  Synthetic Construct
caggtgaagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacagtgtttatgcagatggactctctgcgggccgaggataccgccgtgtactattgccaccgcaatttctacgacggcagcggcccttttgattattggggccagggcaccctggtgacagtgagctccgccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggaccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacgaggccctgcacaatcactacacacagaagagcctgtccctgtctcctggcaagtgataa

SEQ ID NO: 140 - protein, 450 aa, Artificial Sequence
  Synthetic Construct
QVKLVESGGGVVQPGRSLRLSCEASGFIFSTYGMHWVRQAPGKGLEWVAVIWFDGSNIYYADSVKGRFTISRDNSKNTVFMQMDSLRAEDTAVYYCHRNFYDGSGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - DNA, 646 bp, Artificial Sequence
  Synthetic Construct
cgacatccagatgacccagtctccaagcacactgtccgcctctgtgggcgatagggtgaccatcacatgcagagccagccagtttatctcccggtggctggcctggtaccagcagaagccaggcaaggcccccaagctgctgatctataaggccagctccctggagtctggagtgcctagccgcttcagcggctccggctctgagacacactttaccctgacaatctctagcctgcagccagacgatgtggccacctactattgccaggagtacacctcctatggcaggacattcggccagggcaccaaggtggagatcaagacagtggccgcccccagcgtgttcatctttccccctagcgacgagcagctgaagtccggcaccgcctctgtggtgtgcctgctgaacaacttctaccctcgggaggccaaggtgcagtggaaggtggataacgccctgcagagcggcaattcccaggagtctgtgacagagcaggacagcaaggattccacctattctctgtcctctaccctgacactgtccaaggccgactacgagaagcacaaggtgtatgcctgcgaggtgacacaccagggcctgagctcccccgtgaccaagagcttcaacaggggcgagtgttgataa

SEQ ID NO: 142 - protein, 213 aa, Artificial Sequence
  Synthetic Construct
DIQMTQSPSTLSASVGDRVTITCRASQFISRWLAWYQQKPGKAPKLLIYKASSLESGVPSRFSGSGSETHFTLTISSLQPDDVATYYCQEYTSYGRTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - DNA, 360 bp, Artificial Sequence
  Synthetic Construct
caggtgaagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacagtgtttatgcagatggactctctgcgggccgaggataccgccgtgtactattgccaccgcaatttctacgacggcagcggcccttttgattattggggccagggcaccctggtgacagtgagctcc

SEQ ID NO: 144 - protein, 120 aa, Artificial Sequence
  Synthetic Construct
QVKLVESGGGVVQPGRSLRLSCEASGFIFSTYGMHWVRQAPGKGLEWVAVIWFDGSNIYYADSVKGRFTISRDNSKNTVFMQMDSLRAEDTAVYYCHRNFYDGSGPFDYWGQGTLVTVSS

SEQ ID NO: 145 - DNA, 322 bp, Artificial Sequence
  Synthetic Construct
cgacatccagatgacccagtctccaagcacactgtccgcctctgtgggcgatagggtgaccatcacatgcagagccagccagtttatctcccggtggctggcctggtaccagcagaagccaggcaaggcccccaagctgctgatctataaggccagctccctggagtctggagtgcctagccgcttcagcggctccggctctgagacacactttaccctgacaatctctagcctgcagccagacgatgtggccacctactattgccaggagtacacctcctatggcaggacattcggccagggcaccaaggtggagatcaag

SEQ ID NO: 146 - protein, 107 aa, Artificial Sequence
  Synthetic Construct
DIQMTQSPSTLSASVGDRVTITCRASQFISRWLAWYQQKPGKAPKLLIYKASSLESGVPSRFSGSGSETHFTLTISSLQPDDVATYYCQEYTSYGRTFGQGTKVEIK

SEQ ID NO: 147 - DNA, 996 bp, Artificial Sequence
  Synthetic Construct
gccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggaccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacgaggccctgcacaatcactacacacagaagagcctgtccctgtctcctggcaagtgataa

SEQ ID NO: 148 - protein, 330 aa, Artificial Sequence [sequence omitted]

SEQ ID NO: 149 - DNA, 324 bp, Artificial Sequence
  Synthetic Construct
acagtggccgcccccagcgtgttcatctttccccctagcgacgagcagctgaagtccggcaccgcctctgtggtgtgcctgctgaacaacttctaccctcgggaggccaaggtgcagtggaaggtggataacgccctgcagagcggcaattcccaggagtctgtgacagagcaggacagcaaggattccacctattctctgtcctctaccctgacactgtccaaggccgactacgagaagcacaaggtgtatgcctgcgaggtgacacaccagggcctgagctcccccgtgaccaagagcttcaacaggggcgagtgttgataa

SEQ ID NO: 150 - protein, 106 aa, Artificial Sequence
  Synthetic Construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - DNA, 2199 bp, Artificial Sequence
  Synthetic Construct
atggactggacctggagaatcctgttcctggtggcagcagcaaccggaacacacgcccaggtgaagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacagtgtttatgcagatggactctctgcgggccgaggataccgccgtgtactattgccaccgcaatttctacgacggcagcggcccttttgattattggggccagggcaccctggtgacagtgagctccgccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggaccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacgaggccctgcacaatcactacacacagaagagcctgtccctgtctcctggcaagaggggaaggaagaggagaagcggctccggcgccacaaacttcagcctgctgaagcaggcaggcgatgtggaggagaatccaggacctatggtgctgcagacccaggtgttcatcagcctgctgctgtggatctccggcgcctacggcgacatccagatgacccagtctccaagcacactgtccgcctctgtgggcgatagggtgaccatcacatgcagagccagccagtttatctcccggtggctggcctggtaccagcagaagccaggcaaggcccccaagctgctgatctataaggccagctccctggagtctggagtgcctagccgcttcagcggctccggctctggcacagagtttaccctgacaatctctagcctgcagccagacgatttcgccacctactattgccaggagtacacctcctatggcaggacattcggccagggcaccaaggtggagatcaagacagtggccgcccccagcgtgttcatctttccccctagcgacgagcagctgaagtccggcaccgcctctgtggtgtgcctgctgaacaacttctaccctcgggaggccaaggtgcagtggaaggtggataacgccctgcagagcggcaattcccaggagtctgtgacagagcaggacagcaaggattccacctattctctgtcctctaccctgacactgtccaaggccgactacgagaagcacaaggtgtatgcctgcgaggtgacacaccagggcctgagctcccccgtgaccaagagcttcaacaggggcgagtgttgataa

SEQ ID NO: 152 - protein, 731 aa, Artificial Sequence
  Synthetic Construct
MDWTWRILFLVAAATGTHAQVKLVESGGGVVQPGRSLRLSCEASGFIFSTYGMHWVRQAPGKGLEWVAVIWFDGSNIYYADSVKGRFTISRDNSKNTVFMQMDSLRAEDTAVYYCHRNFYDGSGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSTLSASVGDRVTITCRASQFISRWLAWYQQKPGKAPKLLIYKASSLESGVPSRFSGSGSGTEFTLTISSLQPDDFATYYCQEYTSYGRTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - DNA, 646 bp, Artificial Sequence
  Synthetic Construct
cgacatccagatgacccagtctccaagcacactgtccgcctctgtgggcgatagggtgaccatcacatgcagagccagccagtttatctcccggtggctggcctggtaccagcagaagccaggcaaggcccccaagctgctgatctataaggccagctccctggagtctggagtgcctagccgcttcagcggctccggctctggcacagagtttaccctgacaatctctagcctgcagccagacgatttcgccacctactattgccaggagtacacctcctatggcaggacattcggccagggcaccaaggtggagatcaagacagtggccgcccccagcgtgttcatctttccccctagcgacgagcagctgaagtccggcaccgcctctgtggtgtgcctgctgaacaacttctaccctcgggaggccaaggtgcagtggaaggtggataacgccctgcagagcggcaattcccaggagtctgtgacagagcaggacagcaaggattccacctattctctgtcctctaccctgacactgtccaaggccgactacgagaagcacaaggtgtatgcctgcgaggtgacacaccagggcctgagctcccccgtgaccaagagcttcaacaggggcgagtgttgataa

SEQ ID NO: 154 - protein, 213 aa, Artificial Sequence
  Synthetic Construct
DIQMTQSPSTLSASVGDRVTITCRASQFISRWLAWYQQKPGKAPKLLIYKASSLESGVPSRFSGSGSGTEFTLTISSLQPDDFATYYCQEYTSYGRTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - DNA, 322 bp, Artificial Sequence
  Synthetic Construct
cgacatccagatgacccagtctccaagcacactgtccgcctctgtgggcgatagggtgaccatcacatgcagagccagccagtttatctcccggtggctggcctggtaccagcagaagccaggcaaggcccccaagctgctgatctataaggccagctccctggagtctggagtgcctagccgcttcagcggctccggctctggcacagagtttaccctgacaatctctagcctgcagccagacgatttcgccacctactattgccaggagtacacctcctatggcaggacattcggccagggcaccaaggtggagatcaag

SEQ ID NO: 156 - protein, 107 aa, Artificial Sequence
  Synthetic Construct
DIQMTQSPSTLSASVGDRVTITCRASQFISRWLAWYQQKPGKAPKLLIYKASSLESGVPSRFSGSGSGTEFTLTISSLQPDDFATYYCQEYTSYGRTFGQGTKVEIK

SEQ ID NO: 157 - DNA, 324 bp, Artificial Sequence; identical to SEQ ID NO: 149
  Synthetic Construct

SEQ ID NO: 158 - protein, 106 aa, Artificial Sequence; identical to SEQ ID NO: 150
  Synthetic Construct

SEQ ID NO: 159 - DNA, 2199 bp, Artificial Sequence
  Synthetic Construct
atggactggacctggagaatcctgttcctggtggcagcagcaaccggaacacacgcccaggtgcagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacactgtacctgcagatgaactctctgcgggccgaggataccgccgtgtactattgccaccgcaatttctacgacggcagcggcccttttgattattggggccagggcaccctggtgacagtgagctccgccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggaccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacgaggccctgcacaatcactacacacagaagagcctgtccctgtctcctggcaagaggggaaggaagaggagaagcggctccggcgccacaaacttcagcctgctgaagcaggcaggcgatgtggaggagaatccaggacctatggtgctgcagacccaggtgttcatcagcctgctgctgtggatctccggcgcctacggcgacatccagatgacccagtctccaagcacactgtccgcctctgtgggcgatagggtgaccatcacatgcagagccagccagtttatctcccggtggctggcctggtaccagcagaagccaggcaaggcccccaagctgctgatctataaggccagctccctggagtctggagtgcctagccgcttcagcggctccggctctgagacacactttaccctgacaatctctagcctgcagccagacgatgtggccacctactattgccaggagtacacctcctatggcaggacattcggccagggcaccaaggtggagatcaagacagtggccgcccccagcgtgttcatctttccccctagcgacgagcagctgaagtccggcaccgcctctgtggtgtgcctgctgaacaacttctaccctcgggaggccaaggtgcagtggaaggtggataacgccctgcagagcggcaattcccaggagtctgtgacagagcaggacagcaaggattccacctattctctgtcctctaccctgacactgtccaaggccgactacgagaagcacaaggtgtatgcctgcgaggtgacacaccagggcctgagctcccccgtgaccaagagcttcaacaggggcgagtgttgataa

SEQ ID NO: 160 - protein, 731 aa, Artificial Sequence
  Synthetic Construct
MDWTWRILFLVAAATGTHAQVQLVESGGGVVQPGRSLRLSCEASGFIFSTYGMHWVRQAPGKGLEWVAVIWFDGSNIYYADSVKGRFTISRDNSKNTLYLQMNSLRAEDTAVYYCHRNFYDGSGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSTLSASVGDRVTITCRASQFISRWLAWYQQKPGKAPKLLIYKASSLESGVPSRFSGSGSETHFTLTISSLQPDDVATYYCQEYTSYGRTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - DNA, 1356 bp, Artificial Sequence
  Synthetic Construct
caggtgcagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacactgtacctgcagatgaactctctgcgggccgaggataccgccgtgtactattgccaccgcaatttctacgacggcagcggcccttttgattattggggccagggcaccctggtgacagtgagctccgccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggaccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacgaggccctgcacaatcactacacacagaagagcctgtccctgtctcctggcaagtgataa

SEQ ID NO: 162 - protein, 450 aa, Artificial Sequence
  Synthetic Construct
QVQLVESGGGVVQPGRSLRLSCEASGFIFSTYGMHWVRQAPGKGLEWVAVIWFDGSNIYYADSVKGRFTISRDNSKNTLYLQMNSLRAEDTAVYYCHRNFYDGSGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - DNA, 360 bp, Artificial Sequence
  Synthetic Construct
caggtgcagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacactgtacctgcagatgaactctctgcgggccgaggataccgccgtgtactattgccaccgcaatttctacgacggcagcggcccttttgattattggggccagggcaccctggtgacagtgagctcc

SEQ ID NO: 164 - protein, 120 aa, Artificial Sequence
  Synthetic Construct
QVQLVESGGGVVQPGRSLRLSCEASGFIFSTYGMHWVRQAPGKGLEWVAVIWFDGSNIYYADSVKGRFTISRDNSKNTLYLQMNSLRAEDTAVYYCHRNFYDGSGPFDYWGQGTLVTVSS

SEQ ID NO: 165 - DNA, 996 bp, Artificial Sequence; identical to SEQ ID NO: 147
  Synthetic Construct

SEQ ID NO: 166 - protein, 330 aa, Artificial Sequence [sequence omitted]

SEQ ID NO: 167 - DNA, 2199 bp, Artificial Sequence
  Synthetic Construct
atggactggacctggagaatcctgttcctggtggcagcagcaaccggaacacacgcccaggtgcagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacactgtacctgcagatgaactctctgcgggccgaggataccgccgtgtactattgccaccgcaatttctacgacggcagcggcccttttgattattggggccagggcaccctggtgacagtgagctccgccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggaccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacgaggccctgcacaatcactacacacagaagagcctgtccctgtctcctggcaagaggggaaggaagaggagaagcggctccggcgccacaaacttcagcctgctgaagcaggcaggcgatgtggaggagaatccaggacctatggtgctgcagacccaggtgttcatcagcctgctgctgtggatctccggcgcctacggcgacatccagatgacccagtctccaagcacactgtccgcctctgtgggcgatagggtgaccatcacatgcagagccagccagtttatctcccggtggctggcctggtaccagcagaagccaggcaaggcccccaagctgctgatctataaggccagctccctggagtctggagtgcctagccgcttcagcggctccggctctggcacagagtttaccctgacaatctctagcctgcagccagacgatttcgccacctactattgccaggagtacacctcctatggcaggacattcggccagggcaccaaggtggagatcaagacagtggccgcccccagcgtgttcatctttccccctagcgacgagcagctgaagtccggcaccgcctctgtggtgtgcctgctgaacaacttctaccctcgggaggccaaggtgcagtggaaggtggataacgccctgcagagcggcaattcccaggagtctgtgacagagcaggacagcaaggattccacctattctctgtcctctaccctgacactgtccaaggccgactacgagaagcacaaggtgtatgcctgcgaggtgacacaccagggcctgagctcccccgtgaccaagagcttcaacaggggcgagtgttgataa

SEQ ID NO: 168 - protein, 731 aa, Artificial Sequence
  Synthetic Construct
MDWTWRILFLVAAATGTHAQVQLVESGGGVVQPGRSLRLSCEASGFIFSTYGMHWVRQAPGKGLEWVAVIWFDGSNIYYADSVKGRFTISRDNSKNTLYLQMNSLRAEDTAVYYCHRNFYDGSGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSTLSASVGDRVTITCRASQFISRWLAWYQQKPGKAPKLLIYKASSLESGVPSRFSGSGSGTEFTLTISSLQPDDFATYYCQEYTSYGRTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - DNA, 2199 bp, Artificial Sequence
  Synthetic Construct
atggactggacctggagaatcctgttcctggtggcagcagcaaccggaacacacgcccaggtgaagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacagtgtttatgcagatggactctctgcgggccgaggataccgccgtgtactattgccaccgcaatttctacgacggcagcggcccttttgattattggggccagggcaccctggtgacagtgagctccgccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggaccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacggcgccctgcacaatcactacacacagaagagcctgtccctgtctcctggcaagaggggaaggaagaggagaagcggctccggcgccacaaacttcagcctgctgaagcaggcaggcgatgtggaggagaatccaggacctatggtgctgcagacccaggtgttcatcagcctgctgctgtggatctccggcgcctacggcgacatccagatgacccagtctccaagcacactgtccgcctctgtgggcgatagggtgaccatcacatgcagagccagccagtttatctcccggtggctggcctggtaccagcagaagccaggcaaggcccccaagctgctgatctataaggccagctccctggagtctggagtgcctagccgcttcagcggctccggctctgagacacactttaccctgacaatctctagcctgcagccagacgatgtggccacctactattgccaggagtacacctcctatggcaggacattcggccagggcaccaaggtggagatcaagacagtggccgcccccagcgtgttcatctttccccctagcgacgagcagctgaagtccggcaccgcctctgtggtgtgcctgctgaacaacttctaccctcgggaggccaaggtgcagtggaaggtggataacgccctgcagagcggcaattcccaggagtctgtgacagagcaggacagcaaggattccacctattctctgtcctctaccctgacactgtccaaggccgactacgagaagcacaaggtgtatgcctgcgaggtgacacaccagggcctgagctcccccgtgaccaagagcttcaacaggggcgagtgttgataa

SEQ ID NO: 170 - protein, 731 aa, Artificial Sequence
  Synthetic Construct
MDWTWRILFLVAAATGTHAQVKLVESGGGVVQPGRSLRLSCEASGFIFSTYGMHWVRQAPGKGLEWVAVIWFDGSNIYYADSVKGRFTISRDNSKNTVFMQMDSLRAEDTAVYYCHRNFYDGSGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGKRGRKRRSGSGATNFSLLKQAGDVEENPGPMVLQTQVFISLLLWISGAYGDIQMTQSPSTLSASVGDRVTITCRASQFISRWLAWYQQKPGKAPKLLIYKASSLESGVPSRFSGSGSETHFTLTISSLQPDDVATYYCQEYTSYGRTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - DNA, 1356 bp, Artificial Sequence
  Synthetic Construct
caggtgaagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacagtgtttatgcagatggactctctgcgggccgaggataccgccgtgtactattgccaccgcaatttctacgacggcagcggcccttttgattattggggccagggcaccctggtgacagtgagctccgccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggaccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacggcgccctgcacaatcactacacacagaagagcctgtccctgtctcctggcaagtgataa

SEQ ID NO: 172 - protein, 450 aa, Artificial Sequence
  Synthetic Construct
QVKLVESGGGVVQPGRSLRLSCEASGFIFSTYGMHWVRQAPGKGLEWVAVIWFDGSNIYYADSVKGRFTISRDNSKNTVFMQMDSLRAEDTAVYYCHRNFYDGSGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 173 - DNA, 360 bp, Artificial Sequence; identical to SEQ ID NO: 143
  Synthetic Construct

SEQ ID NO: 174 - protein, 120 aa, Artificial Sequence; identical to SEQ ID NO: 144
  Synthetic Construct

SEQ ID NO: 175 - DNA, 996 bp, Artificial Sequence
  Synthetic Construct
gccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggaccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacggcgccctgcacaatcactacacacagaagagcctgtccctgtctcctggcaagtgataa

SEQ ID NO: 176 - protein, 330 aa, Artificial Sequence; identical to SEQ ID NO: 124
  Synthetic Construct

SEQ ID NO: 177 - DNA, 2199 bp, Artificial Sequence
  Synthetic Construct
atggactggacctggagaatcctgttcctggtggcagcagcaaccggaacacacgcccaggtgaagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacagtgtttatgcagatggactctctgcgggccgaggataccgccgtgtactattgccaccgcaatttctacgacggcagcggcccttttgattattggggccagggcaccctggtgacagtgagctccgccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggaccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacggcgccctgcacaatcactacacacagaagagcctgtccctgtctcctggcaagaggggaaggaagaggagaagcggctccggcgccacaaacttcagcctgctgaagcaggcaggcgatgtggaggagaatccaggacctatggtgctgcagacccaggtgttcatcagcctgctgctgtggatctccggcgcctacggcgacatccagatgacccagtctccaagcacactgtccgcctctgtgggcgatagggtgaccatcacatgcagagccagccagtttatctcccggtggctggcctggtaccagcagaagccaggcaaggcccccaagctgctgatctataaggccagctccctggagtctggagtgcctagccgcttcagcggctccggctctggcacagagtttaccctgacaatctctagcctgcagccagacgatttcgccacctactattgccaggagtacacctcctatggcaggacattcggccagggcaccaaggtggagatcaagacagtggccgcccccagcgtgttcatctttccccctagcgacgagcagctgaagtccggcaccgcctctgtggtgtgcctgctgaacaacttctaccctcgggaggccaaggtgcagtggaaggtggataacgccctgcagagcggcaattcccaggagtctgtgacagagcaggacagcaaggattccacctattctctgtcctctaccctgacactgtccaaggccgactacgagaagcacaaggtgtatgcctgcgaggtgacacaccagggcctgagctcccccgtgaccaagagcttcaacaggggcgagtgttgataa

SEQ ID NO: 178 - protein, 731 aa, Artificial Sequence
  Synthetic Construct
MDWTWRILFLVAAATGTHAQVKLVESGGGVVQPGRSLRLSCEASGFIFSTYGMHWVRQAPGKGLEWVAVIWFDGSNIYYADSVKGRFTISRDNSKNTVFMQMDSLRAEDTAVYYCHRNFYDGSGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGKRGRKRRSGSGATNFSLLKQAGDVEENPGPMVLQTQVFISLLLWISGAYGDIQMTQSPSTLSASVGDRVTITCRASQFISRWLAWYQQKPGKAPKLLIYKASSLESGVPSRFSGSGSGTEFTLTISSLQPDDFATYYCQEYTSYGRTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 179 - DNA, 2199 bp, Artificial Sequence
  Synthetic Construct
atggactggacctggagaatcctgttcctggtggcagcagcaaccggaacacacgcccaggtgcagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacactgtacctgcagatgaactctctgcgggccgaggataccgccgtgtactattgccaccgcaatttctacgacggcagcggcccttttgattattggggccagggcaccctggtgacagtgagctccgccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggaccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacggcgccctgcacaatcactacacacagaagagcctgtccctgtctcctggcaagaggggaaggaagaggagaagcggctccggcgccacaaacttcagcctgctgaagcaggcaggcgatgtggaggagaatccaggacctatggtgctgcagacccaggtgttcatcagcctgctgctgtggatctccggcgcctacggcgacatccagatgacccagtctccaagcacactgtccgcctctgtgggcgatagggtgaccatcacatgcagagccagccagtttatctcccggtggctggcctggtaccagcagaagccaggcaaggcccccaagctgctgatctataaggccagctccctggagtctggagtgcctagccgcttcagcggctccggctctgagacacactttaccctgacaatctctagcctgcagccagacgatgtggccacctactattgccaggagtacacctcctatggcaggacattcggccagggcaccaaggtggagatcaagacagtggccgcccccagcgtgttcatctttccccctagcgacgagcagctgaagtccggcaccgcctctgtggtgtgcctgctgaacaacttctaccctcgggaggccaaggtgcagtggaaggtggataacgccctgcagagcggcaattcccaggagtctgtgacagagcaggacagcaaggattccacctattctctgtcctctaccctgacactgtccaaggccgactacgagaagcacaaggtgtatgcctgcgaggtgacacaccagggcctgagctcccccgtgaccaagagcttcaacaggggcgagtgttgataa

SEQ ID NO: 180 - protein, 730 aa, Artificial Sequence
  Synthetic Construct
MDWTWRILFLVAAATGTHAQVQLVESGGGVVQPGRSLRLSCEASGFIFSTYGMHWVRQAPGKGLEWVAVIWFDGSNIYYADSVKGRFTISRDNSKNTLYLQMNSLRAEDTAVYYCHRNFYDGSGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGRGRKRRSGSGATNFSLLKQAGDVEENPGPMVLQTQVFISLLLWISGAYGDIQMTQSPSTLSASVGDRVTITCRASQFISRWLAWYQQKPGKAPKLLIYKASSLESGVPSRFSGSGSETHFTLTISSLQPDDVATYYCQEYTSYGRTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 181 - DNA, 1356 bp, Artificial Sequence
  Synthetic Construct
caggtgcagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacactgtacctgcagatgaactctctgcgggccgaggataccgccgtgtactattgccaccgcaatttctacgacggcagcggcccttttgattattggggccagggcaccctggtgacagtgagctccgccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggaccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacggcgccctgcacaatcactacacacagaagagcctgtccctgtctcctggcaagtgataa

SEQ ID NO: 182 - protein, 450 aa, Artificial Sequence
  Synthetic Construct
QVQLVESGGGVVQPGRSLRLSCEASGFIFSTYGMHWVRQAPGKGLEWVAVIWFDGSNIYYADSVKGRFTISRDNSKNTLYLQMNSLRAEDTAVYYCHRNFYDGSGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 183 - DNA, 360 bp, Artificial Sequence; identical to SEQ ID NO: 163
  Synthetic Construct

SEQ ID NO: 184 - protein, 120 aa, Artificial Sequence; identical to SEQ ID NO: 164
  Synthetic Construct

SEQ ID NO: 185 - DNA, 996 bp, Artificial Sequence; identical to SEQ ID NO: 175
  Synthetic Construct

SEQ ID NO: 186 - protein, 330 aa, Artificial Sequence; identical to SEQ ID NO: 124
  Synthetic Construct

SEQ ID NO: 187 - DNA, 2199 bp, Artificial Sequence
  Synthetic Construct
atggactggacctggagaatcctgttcctggtggcagcagcaaccggaacacacgcccaggtgcagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacactgtacctgcagatgaactctctgcgggccgaggataccgccgtgtactattgccaccgcaatttctacgacggcagcggcccttttgattattggggccagggcaccctggtgacagtgagctccgccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggaccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacggcgccctgcacaatcactacacacagaagagcctgtccctgtctcctggcaagaggggaaggaagaggagaagcggctccggcgccacaaacttcagcctgctgaagcaggcaggcgatgtggaggagaatccaggacctatggtgctgcagacccaggtgttcatcagcctgctgctgtggatctccggcgcctacggcgacatccagatgacccagtctccaagcacactgtccgcctctgtgggcgatagggtgaccatcacatgcagagccagccagtttatctcccggtggctggcctggtaccagcagaagccaggcaaggcccccaagctgctgatctataaggccagctccctggagtctggagtgcctagccgcttcagcggctccggctctggcacagagtttaccctgacaatctctagcctgcagccagacgatttcgccacctactattgccaggagtacacctcctatggcaggacattcggccagggcaccaaggtggagatcaagacagtggccgcccccagcgtgttcatctttccccctagcgacgagcagctgaagtccggcaccgcctctgtggtgtgcctgctgaacaacttctaccctcgggaggccaaggtgcagtggaaggtggataacgccctgcagagcggcaattcccaggagtctgtgacagagcaggacagcaaggattccacctattctctgtcctctaccctgacactgtccaaggccgactacgagaagcacaaggtgtatgcctgcgaggtgacacaccagggcctgagctcccccgtgaccaagagcttcaacaggggcgagtgttgataa

SEQ ID NO: 188 - protein, 731 aa, Artificial Sequence
  Synthetic Construct
MDWTWRILFLVAAATGTHAQVQLVESGGGVVQPGRSLRLSCEASGFIFSTYGMHWVRQAPGKGLEWVAVIWFDGSNIYYADSVKGRFTISRDNSKNTLYLQMNSLRAEDTAVYYCHRNFYDGSGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGKRGRKRRSGSGATNFSLLKQAGDVEENPGPMVLQTQVFISLLLWISGAYGDIQMTQSPSTLSASVGDRVTITCRASQFISRWLAWYQQKPGKAPKLLIYKASSLESGVPSRFSGSGSGTEFTLTISSLQPDDFATYYCQEYTSYGRTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 189 - DNA, 2199 bp, Artificial Sequence
  Synthetic Construct
atggactggacctggagaatcctgttcctggtggcagcagcaaccggaacacacgcccaggtgaagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacagtgtttatgcagatggactctctgcgggccgaggataccgccgtgtactattgccaccgcaatttctacgacggcagcggcccttttgattattggggccagggcaccctggtgacagtgagctccgccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggcccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcgccgtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacgaggccctgcacaatcactacacacagaagagcctgtccctgtctcctggcaagaggggaaggaagaggagaagcggctccggcgccacaaacttcagcctgctgaagcaggcaggcgatgtggaggagaatccaggacctatggtgctgcagacccaggtgttcatcagcctgctgctgtggatctccggcgcctacggcgacatccagatgacccagtctccaagcacactgtccgcctctgtgggcgatagggtgaccatcacatgcagagccagccagtttatctcccggtggctggcctggtaccagcagaagccaggcaaggcccccaagctgctgatctataaggccagctccctggagtctggagtgcctagccgcttcagcggctccggctctgagacacactttaccctgacaatctctagcctgcagccagacgatgtggccacctactattgccaggagtacacctcctatggcaggacattcggccagggcaccaaggtggagatcaagacagtggccgcccccagcgtgttcatctttccccctagcgacgagcagctgaagtccggcaccgcctctgtggtgtgcctgctgaacaacttctaccctcgggaggccaaggtgcagtggaaggtggataacgccctgcagagcggcaattcccaggagtctgtgacagagcaggacagcaaggattccacctattctctgtcctctaccctgacactgtccaaggccgactacgagaagcacaaggtgtatgcctgcgaggtgacacaccagggcctgagctcccccgtgaccaagagcttcaacaggggcgagtgttgataa

SEQ ID NO: 190 - protein, 731 aa, Artificial Sequence
  Synthetic Construct
MDWTWRILFLVAAATGTHAQVKLVESGGGVVQPGRSLRLSCEASGFIFSTYGMHWVRQAPGKGLEWVAVIWFDGSNIYYADSVKGRFTISRDNSKNTVFMQMDSLRAEDTAVYYCHRNFYDGSGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEAPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSTLSASVGDRVTITCRASQFISRWLAWYQQKPGKAPKLLIYKASSLESGVPSRFSGSGSETHFTLTISSLQPDDVATYYCQEYTSYGRTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 191 - DNA, 1356 bp, Artificial Sequence
  Synthetic Construct
caggtgaagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacagtgtttatgcagatggactctctgcgggccgaggataccgccgtgtactattgccaccgcaatttctacgacggcagcggcccttttgattattggggccagggcaccctggtgacagtgagctccgccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggcccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcgccgtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacgaggccctgcacaatcactacacacagaagagcctgtccctgtctcctggcaagtgataa

SEQ ID NO: 192 - protein, 450 aa, Artificial Sequence
  Synthetic Construct
QVKLVESGGGVVQPGRSLRLSCEASGFIFSTYGMHWVRQAPGKGLEWVAVIWFDGSNIYYADSVKGRFTISRDNSKNTVFMQMDSLRAEDTAVYYCHRNFYDGSGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEAPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - DNA, 360 bp, Artificial Sequence; identical to SEQ ID NO: 143
  Synthetic Construct

SEQ ID NO: 194 - protein, 120 aa, Artificial Sequence; identical to SEQ ID NO: 144
  Synthetic Construct

SEQ ID NO: 195 - DNA, 996 bp, Artificial Sequence
  Synthetic Construct
gccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggcccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcgccgtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacgaggccctgcacaatcactacacacagaagagcctgtccctgtctcctggcaagtgataa

SEQ ID NO: 196 - protein, 329 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEAPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 197 - DNA, 2199 bp, Artificial Sequence
  Synthetic Construct
atggactggacctggagaatcctgttcctggtggcagcagcaaccggaacacacgcccaggtgaagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacagtgtttatgcagatggactctctgcgggccgaggataccgccgtgtactattgccaccgcaatttctacgacggcagcggcccttttgattattggggccagggcaccctggtgacagtgagctccgccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggcccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcgccgtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacgaggccctgcacaatcactacacacagaagagcctgtccctgtctcctggcaagaggggaaggaagaggagaagcggctccggcgccacaaacttcagcctgctgaagcaggcaggcgatgtggaggagaatccaggacctatggtgctgcagacccaggtgttcatcagcctgctgctgtggatctccggcgcctacggcgacatccagatgacccagtctccaagcacactgtccgcctctgtgggcgatagggtgaccatcacatgcagagccagccagtttatctcccggtggctggcctggtaccagcagaagccaggcaaggcccccaagctgctgatctataaggccagctccctggagtctggagtgcctagccgcttcagcggctccggctctggcacagagtttaccctgacaatctctagcctgcagccagacgatttcgccacctactattgccaggagtacacctcctatggcaggacattcggccagggcaccaaggtggagatcaagacagtggccgcccccagcgtgttcatctttccccctagcgacgagcagctgaagtccggcaccgcctctgtggtgtgcctgctgaacaacttctaccctcgggaggccaaggtgcagtggaaggtggataacgccctgcagagcggcaattcccaggagtctgtgacagagcaggacagcaaggattccacctattctctgtcctctaccctgacactgtccaaggccgactacgagaagcacaaggtgtatgcctgcgaggtgacacaccagggcctgagctcccccgtgaccaagagcttcaacaggggcgagtgttgataa

SEQ ID NO: 198 - protein, 731 aa, Artificial Sequence
  Synthetic Construct
MDWTWRILFLVAAATGTHAQVKLVESGGGVVQPGRSLRLSCEASGFIFSTYGMHWVRQAPGKGLEWVAVIWFDGSNIYYADSVKGRFTISRDNSKNTVFMQMDSLRAEDTAVYYCHRNFYDGSGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEAPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSTLSASVGDRVTITCRASQFISRWLAWYQQKPGKAPKLLIYKASSLESGVPSRFSGSGSGTEFTLTISSLQPDDFATYYCQEYTSYGRTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - DNA, 2199 bp, Artificial Sequence
  Synthetic Construct
atggactggacctggagaatcctgttcctggtggcagcagcaaccggaacacacgcccaggtgcagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacactgtacctgcagatgaactctctgcgggccgaggataccgccgtgtactattgccaccgcaatttctacgacggcagcggcccttttgattattggggccagggcaccctggtgacagtgagctccgccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggcccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcgccgtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacgaggccctgcacaatcactacacacagaagagcctgtccctgtctcctggcaagaggggaaggaagaggagaagcggctccggcgccacaaacttcagcctgctgaagcaggcaggcgatgtggaggagaatccaggacctatggtgctgcagacccaggtgttcatcagcctgctgctgtggatctccggcgcctacggcgacatccagatgacccagtctccaagcacactgtccgcctctgtgggcgatagggtgaccatcacatgcagagccagccagtttatctcccggtggctggcctggtaccagcagaagccaggcaaggcccccaagctgctgatctataaggccagctccctggagtctggagtgcctagccgcttcagcggctccggctctgagacacactttaccctgacaatctctagcctgcagccagacgatgtggccacctactattgccaggagtacacctcctatggcaggacattcggccagggcaccaaggtggagatcaagacagtggccgcccccagcgtgttcatctttccccctagcgacgagcagctgaagtccggcaccgcctctgtggtgtgcctgctgaacaacttctaccctcgggaggccaaggtgcagtggaaggtggataacgccctgcagagcggcaattcccaggagtctgtgacagagcaggacagcaaggattccacctattctctgtcctctaccctgacactgtccaaggccgactacgagaagcacaaggtgtatgcctgcgaggtgacacaccagggcctgagctcccccgtgaccaagagcttcaacaggggcgagtgttgataa

SEQ ID NO: 200 - protein, 731 aa, Artificial Sequence
  Synthetic Construct
MDWTWRILFLVAAATGTHAQVQLVESGGGVVQPGRSLRLSCEASGFIFSTYGMHWVRQAPGKGLEWVAVIWFDGSNIYYADSVKGRFTISRDNSKNTLYLQMNSLRAEDTAVYYCHRNFYDGSGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEAPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSTLSASVGDRVTITCRASQFISRWLAWYQQKPGKAPKLLIYKASSLESGVPSRFSGSGSETHFTLTISSLQPDDVATYYCQEYTSYGRTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - DNA, 1356 bp, Artificial Sequence
  Synthetic Construct
caggtgcagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacactgtacctgcagatgaactctctgcgggccgaggataccgccgtgtactattgccaccgcaatttctacgacggcagcggcccttttgattattggggccagggcaccctggtgacagtgagctccgccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggcccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcgccgtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacgaggccctgcacaatcactacacacagaagagcctgtccctgtctcctggcaagtgataa

SEQ ID NO: 202 - protein, 450 aa, Artificial Sequence
  Synthetic Construct
QVQLVESGGGVVQPGRSLRLSCEASGFIFSTYGMHWVRQAPGKGLEWVAVIWFDGSNIYYADSVKGRFTISRDNSKNTLYLQMNSLRAEDTAVYYCHRNFYDGSGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEAPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - DNA, 360 bp, Artificial Sequence; identical to SEQ ID NO: 163
  Synthetic Construct

SEQ ID NO: 204 - protein, 120 aa, Artificial Sequence; identical to SEQ ID NO: 164
  Synthetic Construct

SEQ ID NO: 205 - DNA, 996 bp, Artificial Sequence; identical to SEQ ID NO: 195
  Synthetic Construct

SEQ ID NO: 206 - protein, 330 aa, Artificial Sequence; identical to SEQ ID NO: 134
  Synthetic Construct

SEQ ID NO: 207 - DNA, 2199 bp, Artificial Sequence
  Synthetic Construct
atggactggacctggagaatcctgttcctggtggcagcagcaaccggaacacacgcccaggtgcagctggtggagagcggcggcggcgtggtgcagccaggcaggtccctgagactgtcttgcgaggccagcggcttcatctttagcacctacggaatgcactgggtgcgccaggcacctggcaagggactggagtgggtggccgtgatctggtttgacggctccaacatctactatgccgattctgtgaagggccggttcaccatctcccgcgacaactctaagaatacactgtacctgcagatgaactctctgcgggccgaggataccgccgtgtactattgccaccgcaatttctacgacggcagcggcccttttgattattggggccagggcaccctggtgacagtgagctccgccagcacaaagggaccatccgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtcctgtgataagacccacacatgccctccctgtcctgcaccagagctgctgggcggcccatccgtgttcctgtttccacccaagcctaaggacaccctgatgatctctaggacccccgaggtgacatgcgtggtggtggacgtgagccacgaggcccccgaggtgaagttcaactggtacgtggatggcgtggaggtgcacaatgccaagacaaagccaagggaggagcagtacaactctacctatagagtggtgagcgtgctgacagtgctgcaccaggactggctgaacggcaaggagtataagtgcgccgtgtccaataaggccctgcccgcccctatcgagaagaccatctctaaggccaagggccagcctagggagccacaggtgtacacactgcctccatccagagacgagctgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtatagcaagctgaccgtggataagtccagatggcagcagggcaacgtgttttcctgttctgtgatgcacgaggccctgcacaatcactacacacagaagagcctgtccctgtctcctggcaagaggggaaggaagaggagaagcggctccggcgccacaaacttcagcctgctgaagcaggcaggcgatgtggaggagaatccaggacctatggtgctgcagacccaggtgttcatcagcctgctgctgtggatctccggcgcctacggcgacatccagatgacccagtctccaagcacactgtccgcctctgtgggcgatagggtgaccatcacatgcagagccagccagtttatctcccggtggctggcctggtaccagcagaagccaggcaaggcccccaagctgctgatctataaggccagctccctggagtctggagtgcctagccgcttcagcggctccggctctggcacagagtttaccctgacaatctctagcctgcagccagacgatttcgccacctactattgccaggagtacacctcctatggcaggacattcggccagggcaccaaggtggagatcaagacagtggccgcccccagcgtgttcatctttccccctagcgacgagcagctgaagtccggcaccgcctctgtggtgtgcctgctgaacaacttctaccctcgggaggccaaggtgcagtggaaggtggataacgccctgcagagcggcaattcccaggagtctgtgacagagcaggacagcaaggattccacctattctctgtcctctaccctgacactgtccaaggccgactacgagaagcacaaggtgtatgcctgcgaggtgacacaccagggcctgagctcccccgtgaccaagagcttcaacaggggcgagtgttgataa

SEQ ID NO: 208 - protein, 731 aa, Artificial Sequence
  Synthetic Construct
MDWTWRILFLVAAATGTHAQVQLVESGGGVVQPGRSLRLSCEASGFIFSTYGMHWVRQAPGKGLEWVAVIWFDGSNIYYADSVKGRFTISRDNSKNTLYLQMNSLRAEDTAVYYCHRNFYDGSGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEAPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSTLSASVGDRVTITCRASQFISRWLAWYQQKPGKAPKLLIYKASSLESGVPSRFSGSGSGTEFTLTISSLQPDDFATYYCQEYTSYGRTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 209 - protein, 114 aa, Artificial Sequence
  Synthetic Construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDAFDIWGQGTMVTVSS

SEQ ID NO: 210 - protein, 111 aa, Artificial Sequence
  Synthetic Construct
QLVLTQSPSASASLGASVKLTCTLSSGHSSYAIAWHQQQPEKGPRYLMKVNSDGSHTKGDGIPDRFSGSSSGAERYLTISSLQSEDEADYYCQTWGTGIWVFGGGTKLTVL

SEQ ID NO: 211 - protein, 114 aa, Artificial Sequence
  Synthetic Construct
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARDAFDIWGFGTMVTVSS

SEQ ID NO: 212 - protein, 121 aa, Artificial Sequence
  Synthetic Construct
QVQLVQSGAEVKKPGASVKVSCKASGYTFTSYAIHWVRQAPGQRLEWMGWIKAGNGNTRYSQKFQDRVTITRDTSTTTAYMELSSLRSEDTAVYYCALLTVLTPDDAFDIWGQGTMVTVSS

SEQ ID NO: 213 - protein, 113 aa, Artificial Sequence
  Synthetic Construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGGGTKVEIK

SEQ ID NO: 214 - protein, 113 aa, Artificial Sequence; identical to SEQ ID NO: 40
  Synthetic Construct

(19) United States
(12) Patent Application Publication
(10) Pub. No.: US 2024/0287166 A1
(43) Pub. Date: Aug. 29, 2024

(54) DNA ANTIBODY CONSTRUCTS FOR USE AGAINST PLASMODIUM PARASITES

(71) Applicants: David Weiner, Merion, PA (US); Nicholas Tursi, Moorestown, NJ (US); Ami Patel, Philadelphia, PA (US)

(72) Inventors: David Weiner, Merion, PA (US); Nicholas Tursi, Moorestown, NJ (US); Ami Patel, Philadelphia, PA (US)

(21) Appl. No.: 18/564,951

(22) PCT Filed: May 27, 2022
(86) PCT No.: PCT/US22/31271

§ 371 Date: Nov. 28, 2023

Related U.S. Application Data
(60) Provisional application No. 63/250,426, filed on Sep. 30, 2021.
(60) Provisional application No. 63/194,268, filed on May 28, 2021.

(51) Int. Cl. C07K 16/20; A61K 39/00; A61K 48/00; A61P 33/06

(52) U.S. Cl. CPC C07K 16/205; A61P 33/06; A61K 2039/505; A61K 48/00; C07K 2317/24; C07K 2317/76

(57) ABSTRACT

Disclosed herein is a composition including a recombinant nucleic acid sequence that encodes an antibody to a Plasmodium parasitic antigen. Also disclosed herein is a method of generating a synthetic antibody in a subject by administering the composition to the subject. The disclosure also provides a method of preventing and/or treating a Plasmodium parasitic infection in a subject using said composition and method of generation.

### CROSS REFERENCE TO RELATED APPLICATIONS

This application claims priority to U.S. Provisional Patent Application No. 63/194,268, filed May 28, 2021 and U.S. Provisional Patent Application No. 63/250,426, filed Sep. 30, 2021, each of which is hereby incorporated by reference herein in its entirety.

### TECHNICAL FIELD

The present invention relates to a composition comprising a recombinant nucleic acid sequence for generating one or more synthetic antibodies, and functional fragments thereof, in vivo, and a method of preventing and/or treating parasitic infection in a subject by administering said composition.

### BACKGROUND

According to the World Health Organization, there were approximately 228 million malaria cases globally, and 405,000 deaths in 2019 (WHO, 2019). The majority of malaria cases occur in Africa, Southeast Asia, and South America (Snow et al., 2005). Multiple species of the Plasmodium parasite, the causative agent of malaria infect humans, with symptoms ranging from fever and chills to anemia, coma, and death (Milner, 2018). In Africa, Plasmodium falciparum: accounts for the majority of malaria cases.

Thus, there is need in the art for improved therapeutics for the prevention and/or treatment of Plasmodium parasite infection. The current invention satisfies this need.

### SUMMARY

In one embodiment, the present invention relates to a nucleic acid molecule comprising one or more nucleotide sequence encoding one or more polypeptide, wherein said one or more polypeptide is one or more selected from the group consisting of: a) a heavy chain polypeptide of an anti-Plasmodium circumsporozoite protein (CSP) antibody; and b) a light chain polypeptide of an anti-Plasmodium CSP antibody.

In one embodiment, said nucleotide sequence encoding the heavy chain polypeptide comprises a nucleotide sequence encoding a variable heavy chain region, wherein said variable heavy chain region comprises an amino acid sequence that is at least 95% identical to an amino acid sequence selected from the group consisting of: SEQ ID NO: 8, SEQ ID NO: 22, SEQ ID NO: 38, SEQ ID NO: 58, SEQ ID NO: 76, SEQ ID NO: 100, SEQ ID NO: 114, SEQ ID NO: 122, SEQ ID NO: 132, SEQ ID NO: 144, SEQ ID NO: 164, SEQ ID NO: 174, SEQ ID NO: 184, SEQ ID NO: 194, and SEQ ID NO: 204. In one embodiment, said nucleotide sequence encoding a variable heavy chain region comprises a nucleotide sequence that is at least 95% identical to a nucleotide sequence selected from the group consisting of: SEQ ID NO: 7, SEQ ID NO: 21, SEQ ID NO: 37, SEQ ID NO: 57, SEQ ID NO: 75, SEQ ID NO: 99, SEQ ID NO: 113, SEQ ID NO: 121, SEQ ID NO: 131, SEQ ID NO: 143, SEQ ID NO: 163, SEQ ID NO: 173, SEQ ID NO: 183, SEQ ID NO: 193, and SEQ ID NO: 203.

In one embodiment, said heavy chain polypeptide comprises an amino acid sequence that is at least 95% identical to an amino acid sequence selected from the group consisting of: SEQ ID NO: 4, SEQ ID NO: 18, SEQ ID NO: 26, SEQ ID NO: 34, SEQ ID NO: 46, SEQ ID NO: 54, SEQ ID NO: 64, SEQ ID NO: 72, SEQ ID NO: 80, SEQ ID NO: 96, SEQ ID NO: 112, SEQ ID NO: 120, SEQ ID NO: 130, SEQ ID NO: 140, SEQ ID NO: 162, SEQ ID NO: 172, SEQ ID NO: 182, SEQ ID NO: 192, and SEQ ID NO: 202. In one embodiment, said nucleotide sequence encoding the heavy chain polypeptide comprises a nucleotide sequence that is at least 95% identical to a nucleotide sequence selected from the group consisting of: SEQ ID NO: 3, SEQ ID NO: 17, SEQ ID NO: 25, SEQ ID NO: 33, SEQ ID NO: 45, SEQ ID NO: 53, SEQ ID NO: 63, SEQ ID NO: 71, SEQ ID NO: 79, SEQ ID NO: 95, SEQ ID NO: 111, SEQ ID NO: 119, SEQ ID NO: 129, SEQ ID NO: 139, SEQ ID NO: 161, SEQ ID NO: 171, SEQ ID NO: 181, SEQ ID NO: 191, and SEQ ID NO: 201.

In one embodiment, said nucleotide sequence encoding the light chain polypeptide comprises a nucleotide sequence encoding a variable light chain region, wherein said variable light chain region comprises an amino acid sequence that is at least 95% identical to an amino acid sequence selected from the group consisting of: SEQ ID NO: 10, SEQ ID NO: 30, SEQ ID NO: 40, SEQ ID NO: 50, SEQ ID NO: 68, SEQ ID NO: 102, SEQ ID NO: 108, SEQ ID NO: 146, and SEQ ID NO: 156. In one embodiment, said nucleotide sequence encoding a variable light chain region comprises a nucleotide sequence that is at least 95% identical to a nucleotide sequence selected from the group consisting of: SEQ ID NO: 9, SEQ ID NO: 29, SEQ ID NO: 39, SEQ ID NO: 49, SEQ ID NO: 67, SEQ ID NO: 101, SEQ ID NO: 107, SEQ ID NO: 145, and SEQ ID NO: 155.

In one embodiment, said light chain polypeptide comprises an amino acid sequence that is at least 95% identical to an amino acid sequence selected from the group consisting of SEQ ID NO: 6, SEQ ID NO: 20, SEQ ID NO: 28, SEQ ID NO: 36, SEQ ID NO: 48, SEQ ID NO: 56, SEQ ID NO: 66, SEQ ID NO: 74, SEQ ID NO: 82, SEQ ID NO: 98, SEQ ID NO: 106, SEQ ID NO: 142, and SEQ ID NO: 154. In one embodiment, said nucleotide sequence encoding the light chain polypeptide comprises a nucleotide sequence that is at least 95% identical to a nucleotide sequence selected from the group consisting of: SEQ ID NO: 5, SEQ ID NO: 19, SEQ ID NO: 27, SEQ ID NO: 35, SEQ ID NO: 47, SEQ ID NO: 55, SEQ ID NO: 65, SEQ ID NO: 73, SEQ ID NO: 81, SEQ ID NO: 97, SEQ ID NO: 105, SEQ ID NO: 141, and SEQ ID NO: 153.

In one embodiment, the present invention relates to a composition comprising at least two nucleic acid molecules wherein: a) at least one nucleic acid molecule comprises a nucleotide sequence encoding a heavy chain polypeptide of an anti-Plasmodium CSP antibody; and b) at least one nucleic acid molecule comprises a nucleotide sequence encoding a light chain polypeptide of an anti-Plasmodium CSP antibody.

In one embodiment, said nucleotide sequence encoding the heavy chain polypeptide comprises a nucleotide sequence encoding a variable heavy chain region, wherein said variable heavy chain region comprises an amino acid sequence that is at least 95% identical to an amino acid sequence selected from the group consisting of: SEQ ID NO: 8, SEQ ID NO: 22, SEQ ID NO: 38, SEQ ID NO: 58, SEQ ID NO: 76, SEQ ID NO: 100, SEQ ID NO: 114, SEQ ID NO: 122, SEQ ID NO: 132, SEQ ID NO: 144, SEQ ID NO: 164, SEQ ID NO: 174, SEQ ID NO: 184, SEQ ID NO: 194, and SEQ ID NO: 204. In one embodiment, said nucleotide sequence encoding a variable heavy chain region comprises a nucleotide sequence that is at least 95% identical to a nucleotide sequence selected from the group consisting of: SEQ ID NO: 7, SEQ ID NO: 21, SEQ ID NO: 37, SEQ ID NO: 57, SEQ ID NO: 75, SEQ ID NO: 99, SEQ ID NO: 113, SEQ ID NO: 121, SEQ ID NO: 131, SEQ ID NO: 143, SEQ ID NO: 163, SEQ ID NO: 173, SEQ ID NO: 183, SEQ ID NO: 193, and SEQ ID NO: 203.

In one embodiment, said heavy chain polypeptide comprises an amino acid sequence that is at least 95% identical to an amino acid sequence selected from the group consisting of: SEQ ID NO: 4, SEQ ID NO: 18, SEQ ID NO: 26, SEQ ID NO: 34, SEQ ID NO: 46, SEQ ID NO: 54, SEQ ID NO: 64, SEQ ID NO: 72, SEQ ID NO: 80, SEQ ID NO: 96, SEQ ID NO: 112, SEQ ID NO: 120, SEQ ID NO: 130, SEQ ID NO: 140, SEQ ID NO: 162, SEQ ID NO: 172, SEQ ID NO: 182, SEQ ID NO: 192, and SEQ ID NO: 202. In one embodiment, said nucleotide sequence encoding the heavy chain polypeptide comprises a nucleotide sequence that is at least 95% identical to a nucleotide sequence selected from the group consisting of: SEQ ID NO: 3, SEQ ID NO: 17, SEQ ID NO: 25, SEQ ID NO: 33, SEQ ID NO: 45, SEQ ID NO: 53, SEQ ID NO: 63, SEQ ID NO: 71, SEQ ID NO: 79, SEQ ID NO: 95, SEQ ID NO: 111, SEQ ID NO: 119, SEQ ID NO: 129, SEQ ID NO: 139, SEQ ID NO: 161, SEQ ID NO: 171, SEQ ID NO: 181, SEQ ID NO: 191, and SEQ ID NO: 201.

In one embodiment, said nucleotide sequence encoding the light chain polypeptide comprises a nucleotide sequence encoding a variable light chain region, wherein said variable light chain region comprises an amino acid sequence that is at least 95% identical to an amino acid sequence selected from the group consisting of: SEQ ID NO: 10, SEQ ID NO: 30, SEQ ID NO: 40, SEQ ID NO: 50, SEQ ID NO: 68, SEQ ID NO: 102, SEQ ID NO: 108, SEQ ID NO: 146, and SEQ ID NO: 156. In one embodiment, said nucleotide sequence encoding a variable light chain region comprises a nucleotide sequence that is at least 95% identical to a nucleotide sequence selected from the group consisting of: SEQ ID NO: 9, SEQ ID NO: 29, SEQ ID NO: 39, SEQ ID NO: 49, SEQ ID NO: 67, SEQ ID NO: 101, SEQ ID NO: 107, SEQ ID NO: 145, and SEQ ID NO: 155.

In one embodiment, said light chain polypeptide comprises an amino acid sequence that is at least 95% identical to an amino acid sequence selected from the group consisting of: SEQ ID NO: 6, SEQ ID NO: 20, SEQ ID NO: 28, SEQ ID NO: 36, SEQ ID NO: 48, SEQ ID NO: 56, SEQ ID NO: 66, SEQ ID NO: 74, SEQ ID NO: 82, SEQ ID NO: 98, SEQ ID NO: 106, SEQ ID NO: 142, and SEQ ID NO: 154. In one embodiment, said nucleotide sequence encoding the light chain polypeptide comprises a nucleotide sequence that is at least 95% identical to a nucleotide sequence selected from the group consisting of: SEQ ID NO: 5, SEQ ID NO: 19, SEQ ID NO: 27, SEQ ID NO: 35, SEQ ID NO: 47, SEQ ID NO: 55, SEQ ID NO: 65, SEQ ID NO: 73, SEQ ID NO: 81, SEQ ID NO: 97, SEQ ID NO: 105, SEQ ID NO: 141, and SEQ ID NO: 153.

In one embodiment, the present invention relates to a method of treating or preventing Plasmodium parasitic infection, the method comprising administering to the subject at least one selected from the group consisting of: a) a nucleic acid molecule comprising a nucleotide sequence encoding at least two polypeptides comprising an anti-Plasmodium CSP antibody; and b) a composition comprising at least two nucleic acid molecules comprising at least two nucleotide sequences encoding at least two polypeptides comprising an anti-Plasmodium CSP antibody.

In one embodiment of the method, said at least two polypeptides comprise a heavy chain polypeptide and a light chain polypeptide, wherein said heavy chain polypeptide comprises a variable heavy chain amino sequence at least 95% identical to an amino acid sequence selected from the group consisting of SEQ ID NO: 8, SEQ ID NO: 22, SEQ ID NO: 38, SEQ ID NO: 58, SEQ ID NO: 76, SEQ ID NO: 100, SEQ ID NO: 114, SEQ ID NO: 122, SEQ ID NO: 132, SEQ ID NO: 144, SEQ ID NO: 164, SEQ ID NO: 174, SEQ ID NO: 184, SEQ ID NO: 194, and SEQ ID NO: 204, encoded by a nucleotide sequence that is at least 90% identical to a nucleotide sequence selected from the group consisting of SEQ ID NO: 7, SEQ ID NO: 21, SEQ ID NO: 37, SEQ ID NO: 57, SEQ ID NO: 75, SEQ ID NO: 99, SEQ ID NO: 113, SEQ ID NO: 121, SEQ ID NO: 131, SEQ ID NO: 143, SEQ ID NO: 163, SEQ ID NO: 173, SEQ ID NO: 183, SEQ ID NO: 193, and SEQ ID NO: 203; and wherein said light chain polypeptide comprises a variable light chain amino sequence at least 95% identical to an amino acid sequence selected from the group consisting of SEQ ID NO: 10, SEQ ID NO: 30, SEQ ID NO: 40, SEQ ID NO: 50, SEQ ID NO: 68, SEQ ID NO: 102, SEQ ID NO: 108, SEQ ID NO: 146, and SEQ ID NO: 156, encoded by a nucleotide sequence that is at least 90% identical to a nucleotide sequence selected from the group consisting of SEQ ID NO: 9, SEQ ID NO: 29, SEQ ID NO: 39, SEQ ID NO: 49, SEQ ID NO: 67, SEQ ID NO: 101, SEQ ID NO: 107, SEQ ID NO: 145, and SEQ ID NO: 155.

### BRIEF DESCRIPTION OF THE DRAWINGS

FIG. 1, comprising FIG. 1A through FIG. 1D, depicts MGU10 DMAb sequence alignment, germline genes, and naming schematic. FIG. 1A depicts the plasmid design schematic. FIG. 1B depicts the sequence alignment of MGU10 DMAb constructs to both germline antibody and original clone sequences. Amino acids different from germline shown in blue; amino acids reverted to germline shown in green. Red boxes indicate residues where mutant(s) have germline reversion(s). CDR alignment based on IGMT analysis to germline gene. FIG. 1C depicts a table showing germline variable and joining genes used in sequence alignment. FIG. 1D depicts a table describing DMAb nomenclature in reference to the individual heavy chain (HC) and light chain (LC) mutants.

FIG. 2, comprising FIG. 2A through FIG. 2F, depicts exemplary results demonstrating that in vitro and in vivo produced MGU10 engineered DMAbs express and bind rCSP. FIG. 2A depicts a ribbon diagram of wild type MGU10 VH (purple) and VL (pink) with CDRs (yellow) and all possible germline reversions (red). FIG. 2B depicts a table comparing VH and VL nucleotide sequence differences of the exemplary wild type MGU10 antibody and all MGU10 engineered DMAb (10e) constructs. FIG. 2C depicts exemplary results of reducing SDS-PAGE showing expression of 10e DMAb constructs in Expi293F cell supernatants. FIG. 2D depicts exemplary results of binding ELISA of pooled supernatants harvested for SDS-PAGE analysis in FIG. 2C to rCSP (mean±SD). FIG. 2E depicts exemplary results of in vivo expression of 10e DMAb constructs in BALB/c mice with CD4+ and CD8+ T cell depletion (n=5 mice/group; mean±SEM). FIG. 2F depicts exemplary results of binding ELISA on pooled sera harvested from mice Day 10 post DMAb administration in FIG. 2E to rCSP (mean±SD).

FIG. 3, comprising FIG. 3A through FIG. 3D, depicts CIS43 DMAb sequence alignment, germline genes, and naming schematic. FIG. 3A depicts the plasmid design schematic. FIG. 3B depicts a sequence alignment of CIS43 DMAb constructs to both germline antibody and original clone sequences. Amino acids different from germline shown in blue; amino acids reverted to germline shown in green. Red boxes indicate residues where mutant(s) have germline reversion(s). CDR alignment based on IGMT analysis to germline gene. FIG. 3C depicts a table showing germline variable and joining genes used in sequence alignment. FIG. 3D depicts a table describing DMAb nomenclature in reference to the individual HC and LC mutants.

FIG. 4, comprising FIG. 4A through FIG. 4F, depicts exemplary results demonstrating in vitro and in vivo produced CIS43 engineered DMAbs express and bind rCSP. FIG. 4A depicts a ribbon diagram of wild type CIS43 VH (blue) and VL (teal) with CDRs (yellow) and all possible germline reversions (red). FIG. 4B depicts a table comparing VH and VL nucleotide sequence differences of exemplary wild type CIS43 antibody and all CIS43 (43e) engineered DMAb constructs. FIG. 4C depicts exemplary results of reducing SDS-PAGE showing expression of all six 43e DMAb constructs in Expi293F cell supernatants. FIG. 4D depicts exemplary results of binding ELISA of pooled supernatants harvested for SDS-PAGE analysis in FIG. 4C to rCSP (mean±SD). FIG. 4E depicts exemplary results demonstrating in vivo expression of 43e DMAb constructs in BALB/c mice with CD4+ and CD8+ T cell depletion (n=5 mice/group; data presented as mean±SEM). FIG. 4F depicts exemplary results of binding ELISA on pooled sera harvested from mice Day 10 post DMAb administration in FIG. 4E to rCSP (mean±SD).

FIG. 5, comprising FIG. 5A through FIG. 5D, depicts exemplary results demonstrating that in vivo produced 43e DMAbs express bind rCSP and sporozoites and exhibit a dosing effect. FIG. 5A depicts results demonstrating long term in vivo expression of 43e DMAb-1 and 43e DMAb-2 constructs in BALB/c mice with co-administration of CD4+ and CD8+ T cell depletion (n=5 mice/group; data presented as mean±SEM). FIG. 5B depicts results of binding ELISA performed on pooled sera harvested from mice Day 13 post DMAb administration in FIG. 5A to recombinant CSP (mean±SD). FIG. 5C depicts results of binding ELISA from sera harvest in FIG. 5A to sporozoites. FIG. 5D depicts exemplary results demonstrating long term in vivo expression with T cell depletion of 43e DMAb-2 at varying doses of synthetic DNA as quantified by ELISA.

FIG. 6, comprising FIG. 6A through FIG. 6D, depicts exemplary results demonstrating DMAb down selection and in vivo expression without T cell depletion. FIG. 6A depicts a graphical representation of down selection. FIG. 6B depicts exemplary results of binding ELISA to rCSP of supernatants containing 43e DMAb-4 and 10e DMAb-2. FIG. 6C depicts the quantification of ELISA on sera from BALB/c mice administered varying doses of 43e DMAb-4 and 10e DMAb-2 (n=5 mice/group; mean±SEM). Significance determined by Tukey's multiple comparisons test. **p<0.01; ***p<0.001; ****p<0.0001. FIG. 6D depicts results of binding ELISA to sporozoites of sera from FIG. 6C compared to recombinant mAb 311 positive control.

FIG. 7, comprising FIG. 7A through FIG. 7D, depicts exemplary results demonstrating that 43e DMAb-4 is protective in a rigorous mosquito bite challenge model. FIG. 7A depicts the experimental layout showing DMAb administration, mosquito challenge, and blood smears post challenge. FIG. 7B depicts exemplary IVIS images showing fluorescence intensity of PbPfLuc parasites in the pVAX, mAb 311, and 43e DMAb-4 groups. FIG. 7C depicts exemplary results of percent inhibition of liver infection relative to infection of pVAX control infected mice. FIG. 7D depicts exemplary results of the percent of blood stage parasite free mice, determined through blood smears beginning on day 4 and up to day 10 post challenge. n=5 mice/group. **p<0.01 versus pVAX by Log-rank (Mantel-Cox) test.

FIG. 8, comprising FIG. 8A through FIG. 8B, depicts exemplary results of composite and individual luminescence data. FIG. 8A depicts bar graphs of exemplary grouped luminescence data with individual mice represented as open circles; groups compared to pVAX control mice via Mann Whitney Test, ** p=0.079 where error bars represent standard deviation from the mean. FIG. 8B) depicts a table showing exemplary results of individual luminescence data per mouse (photons/sec).

FIG. 9, comprising FIG. 9A through FIG. 9E, depicts exemplary results demonstrating the protective capacity of different DNA constructs as measured by liver burden in response to parasitic challenge. Groups of 7 mice were immunized on day 0 according to a previously agreed upon protocol with different constructs of DNA displaying different epitopes of Plasmodium falciparum: circumsporozoite protein (CSP), negative control pVax plasmid, or positive control monoclonal antibody AB-311. Three weeks after the immunization, mice were challenged with a transgenic, chimeric Plasmodium berghei parasite expressing the full-length P. falciparum CSP and luciferase (PbPf-GFPLuc). Forty-two hours after challenge, mice were injected with 100 μl of D-Luciferin (30 mg/mL), anesthetized with isoflurane and imaged with the IVIS spectrum to measure the bioluminescence expressed by the chimeric parasites. FIG. 9A depicts a table of each treatment cohort, individual mouse #, individual bioluminescence radiance of D-Luciferin reactant, and the geometric mean for each cohort. FIGS. 9B and 9C depict the bioluminescence liver burden as total flux in photons per second with error bars. FIG. 9D depicts the results expressed as percent inhibition, with the naïve group being considered as 100% of infection. FIG. 9E depicts a summary of the results including the P value, exactness and significance of the Mann-Whitney statistical test in immunized versus naïve cohorts.

FIG. 10 depicts exemplary results demonstrating the protective capacity of different DNA constructs as measured by parasitemia in response to infected mosquito bite challenge. Mice were immunized with the different constructs of DNA displaying different epitopes of Plasmodium falciparum: circumsporozoite protein (CSP), negative control pVax plasmid, or positive control monoclonal antibody AB-311. Twenty-one days later, immunized mice and controls were anesthetized with 2% Avertin and mosquitoes that had previously fed on mice infected with chimeric Plasmodium berghei parasite expressing the full-length P. falciparum CSP were allowed to blood feed on the experimental mice for −10 minutes. The presence of blood-stage parasitemia was then evaluated on days four through ten post-challenge. A “−” indicates the absence of parasitemia, a “+” indicates the detection of parasitemia, and slashes indicate that mice were no longer further evaluated after parasitemia was detected.

### DETAILED DESCRIPTION

The present invention relates to compositions comprising a recombinant nucleic acid sequence encoding an antibody, a fragment thereof, a variant thereof, or a combination thereof. The composition can be administered to a subject in need thereof to facilitate in vivo expression and formation of a synthetic antibody.

In particular, the heavy chain and light chain polypeptides expressed from the recombinant nucleic acid sequences can assemble into the synthetic antibody. The heavy chain polypeptide and the light chain polypeptide can interact with one another such that assembly results in the synthetic antibody being capable of binding the antigen, being more immunogenic as compared to an antibody not assembled as described herein, and being capable of eliciting or inducing an immune response against the antigen.

Additionally, these synthetic antibodies are generated more rapidly in the subject than antibodies that are produced in response to antigen induced immune response. The synthetic antibodies are able to effectively bind and neutralize a range of antigens. The synthetic antibodies are also able to effectively protect against and/or promote survival of disease.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Antigen” refers to proteins that have the ability to generate an immune response in a host. An antigen may be recognized and bound by an antibody. An antigen may originate from within the body or from the external environment.

“Coding sequence” or “encoding nucleic acid” as used herein may mean refers to the nucleic acid (RNA or DNA molecule) that comprise a nucleotide sequence which encodes an antibody as set forth herein. The coding sequence may also comprise a DNA sequence which encodes an RNA sequence. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

“Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse. In some embodiments, the feedback element comprises a controller.

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Endogenous antibody” as used herein may refer to an antibody that is generated in a subject that is administered an effective dose of an antigen for induction of a humoral immune response.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” may mean a polypeptide fragment of an antibody that is function, i.e., can bind to desired target and have the same intended effect as a full length antibody. A fragment of an antibody may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antibody, excluding any heterologous signal peptide added. The fragment may comprise a fragment of a polypeptide that is 95% or more, 96% or more, 97% or more, 98% or more or 99% or more identical to the antibody and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence that encodes an antibody may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Genetic construct” as used herein refers to the DNA or RNA molecules that comprise a nucleotide sequence which encodes a protein, such as an antibody. The genetic construct may also refer to a DNA molecule which transcribes an RNA. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Impedance” as used herein may be used when discussing the feedback mechanism and can be converted to a current value according to Ohm's law, thus enabling comparisons with the preset current.

“Immune response” as used herein may mean the activation of a host's immune system, e.g., that of a mammal, in response to the introduction of one or more nucleic acids and/or peptides. The immune response can be in the form of a cellular or humoral response, or both.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV 40 late promoter and the CMV IE promoter.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Synthetic antibody” as used herein refers to an antibody that is encoded by the recombinant nucleic acid sequence described herein and is generated in a subject.

“Treatment” or “treating,” as used herein can mean protecting of a subject from a disease through means of preventing, suppressing, repressing, or completely eliminating the disease. Preventing the disease involves administering a vaccine of the present invention to a subject prior to onset of the disease. Suppressing the disease involves administering a vaccine of the present invention to a subject after induction of the disease but before its clinical appearance. Repressing the disease involves administering a vaccine of the present invention to a subject after clinical appearance of the disease.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. COMPOSITION

The present invention relates to a composition comprising one or more recombinant nucleic acid sequence encoding an antibody, a fragment thereof, a variant thereof, or a combination thereof. The composition, when administered to a subject in need thereof, can result in the generation of a synthetic antibody in the subject. The synthetic antibody can bind a target molecule (i.e., an antigen) present in the subject. Such binding can neutralize the antigen, block recognition of the antigen by another molecule, for example, a protein or nucleic acid, and elicit or induce an immune response to the antigen.

In one embodiment, the composition comprises one or more nucleic acid molecule comprising one or more nucleotide sequence encoding a synthetic antibody. In one embodiment, said one or more nucleic acid molecule comprises a nucleotide sequence encoding a variable heavy chain region operably linked to a constant heavy chain region. In one embodiment, said one or more nucleic acid molecule comprises a nucleotide sequence encoding a variable light chain region operably linked to a constant light chain region. In one embodiment, said one or more nucleic acid molecule comprises one or more nucleotide sequence encoding a cleavage domain. In one embodiment, said cleavage domain is an enzymatic cleavage domain. In one embodiment, the enzymatic cleavage domain is a furin cleavage domain. In one embodiment, the furin cleavage domain is encoded by a nucleic acid sequence of SEQ ID NO: 83. In one embodiment, the furin cleavage domain comprises the amino acid sequence of SEQ ID NO: 84. In one embodiment, said cleavage domain is a self-cleaving domain. In one embodiment, the self-cleaving domain is a 2A peptide domain. In one embodiment, the 2A peptide domain is encoded by a nucleic acid sequence of SEQ ID NO: 85. In one embodiment, the 2A peptide domain comprises the amino acid sequence of SEQ ID NO: 86.

In one embodiment, the one or more nucleic acid molecule comprises one or more nucleotide sequence encoding an anti-Plasmodium circumsporozoite protein (CSP) antibody.

In one embodiment, the one or more nucleotide sequence encoding an anti-Plasmodium CSP antibody comprises one or more codon optimized nucleic acid sequence encoding an amino acid sequence at least 95%, at least 96%, at least 97%, at least 98%, or at least 99% identical to an amino acid sequence as set forth in one of SEQ ID NO: 2, SEQ ID NO: 16, SEQ ID NO: 24, SEQ ID NO: 32, SEQ ID NO: 44, SEQ ID NO: 52, SEQ ID NO: 62, SEQ ID NO: 70, SEQ ID NO: 78, SEQ ID NO: 94, SEQ ID NO: 104, SEQ ID NO: 110, SEQ ID NO: 116, SEQ ID NO: 118, SEQ ID NO: 126, SEQ ID NO: 128, SEQ ID NO: 136, SEQ ID NO: 138, SEQ ID NO: 152, SEQ ID NO: 160, SEQ ID NO: 168, SEQ ID NO: 170, SEQ ID NO: 178, SEQ ID NO: 180, SEQ ID NO: 188, SEQ ID NO: 190, SEQ ID NO: 198, SEQ ID NO: 200, SEQ ID NO: 208, a variant thereof, or a fragment thereof.

In one embodiment, the one or more nucleotide sequence encoding an anti-Plasmodium CSP antibody comprises one or more codon optimized nucleic acid sequence encoding an amino acid sequence as set forth in one of SEQ ID NO: 2, SEQ ID NO: 16, SEQ ID NO: 24, SEQ ID NO: 32, SEQ ID NO: 44, SEQ ID NO: 52, SEQ ID NO: 62, SEQ ID NO: 70, SEQ ID NO: 78, SEQ ID NO: 94, SEQ ID NO: 104, SEQ ID NO: 110, SEQ ID NO: 116, SEQ ID NO: 118, SEQ ID NO: 126, SEQ ID NO: 128, SEQ ID NO: 136, SEQ ID NO: 138, SEQ ID NO: 152, SEQ ID NO: 160, SEQ ID NO: 168, SEQ ID NO: 170, SEQ ID NO: 178, SEQ ID NO: 180, SEQ ID NO: 188, SEQ ID NO: 190, SEQ ID NO: 198, SEQ ID NO: 200, SEQ ID NO: 208, a variant thereof, or a fragment thereof.

In one embodiment, the one or more nucleotide sequence encoding an anti-Plasmodium CSP antibody comprises one or more RNA sequence transcribed from one or more DNA sequences encoding an amino acid sequence at least 95%, at least 96%, at least 97%, at least 98%, or at least 99% identical to an amino acid sequence as set forth in one of SEQ ID NO: 2, SEQ ID NO: 16, SEQ ID NO: 24, SEQ ID NO: 32, SEQ ID NO: 44, SEQ ID NO: 52, SEQ ID NO: 62, SEQ ID NO: 70, SEQ ID NO: 78, SEQ ID NO: 94, SEQ ID NO: 104, SEQ ID NO: 110, SEQ ID NO: 116, SEQ ID NO: 118, SEQ ID NO: 126, SEQ ID NO: 128, SEQ ID NO: 136, SEQ ID NO: 138, SEQ ID NO: 152, SEQ ID NO: 160, SEQ ID NO: 168, SEQ ID NO: 170, SEQ ID NO: 178, SEQ ID NO: 180, SEQ ID NO: 188, SEQ ID NO: 190, SEQ ID NO: 198, SEQ ID NO: 200, SEQ ID NO: 208, a variant thereof, or a fragment thereof.

In one embodiment, the one or more nucleotide sequence encoding an anti-Plasmodium CSP antibody comprises one or more RNA sequence transcribed from one or more DNA sequences encoding an amino acid sequence as set forth in one of SEQ ID NO: 2, SEQ ID NO: 16, SEQ ID NO: 24, SEQ ID NO: 32, SEQ ID NO: 44, SEQ ID NO: 52, SEQ ID NO: 62, SEQ ID NO: 70, SEQ ID NO: 78, SEQ ID NO: 94, SEQ ID NO: 104, SEQ ID NO: 110, SEQ ID NO: 116, SEQ ID NO: 118, SEQ ID NO: 126, SEQ ID NO: 128, SEQ ID NO: 136, SEQ ID NO: 138, SEQ ID NO: 152, SEQ ID NO: 160, SEQ ID NO: 168, SEQ ID NO: 170, SEQ ID NO: 178, SEQ ID NO: 180, SEQ ID NO: 188, SEQ ID NO: 190, SEQ ID NO: 198, SEQ ID NO: 200, SEQ ID NO: 208, a variant thereof, or a fragment thereof.

In one embodiment, the one or more nucleotide sequence encoding an anti-Plasmodium CSP antibody comprises one or more codon optimized nucleic acid sequences at least 95%, at least 96%, at least 97%, at least 98%, or at least 99% identical to a nucleic acid sequence as set forth in SEQ ID NO: 1, SEQ ID NO: 15, SEQ ID NO: 23, SEQ ID NO: 31, SEQ ID NO: 43, SEQ ID NO: 51, SEQ ID NO: 61, SEQ ID NO: 69, SEQ ID NO: 77, SEQ ID NO: 93, SEQ ID NO: 103, SEQ ID NO: 109, SEQ ID NO: 115, SEQ ID NO: 117, SEQ ID NO: 125, SEQ ID NO: 127, SEQ ID NO: 135, SEQ ID NO: 137, SEQ ID NO: 151, SEQ ID NO: 159, SEQ ID NO: 167, SEQ ID NO: 169, SEQ ID NO: 177, SEQ ID NO: 179, SEQ ID NO: 187, SEQ ID NO: 189, SEQ ID NO: 197, SEQ ID NO: 199, SEQ ID NO: 207, a variant thereof, or a fragment thereof.

In one embodiment, the one or more nucleotide sequence encoding an anti-Plasmodium CSP antibody comprises one or more codon optimized nucleic acid sequences as set forth in SEQ ID NO: 1, SEQ ID NO: 15, SEQ ID NO: 23, SEQ ID NO: 31, SEQ ID NO: 43, SEQ ID NO: 51, SEQ ID NO: 61, SEQ ID NO: 69, SEQ ID NO: 77, SEQ ID NO: 93, SEQ ID NO: 103, SEQ ID NO: 109, SEQ ID NO: 115, SEQ ID NO: 117, SEQ ID NO: 125, SEQ ID NO: 127, SEQ ID NO: 135, SEQ ID NO: 137, SEQ ID NO: 151, SEQ ID NO: 159, SEQ ID NO: 167, SEQ ID NO: 169, SEQ ID NO: 177, SEQ ID NO: 179, SEQ ID NO: 187, SEQ ID NO: 189, SEQ ID NO: 197, SEQ ID NO: 199, SEQ ID NO: 207, a variant thereof, or a fragment thereof.

In one embodiment, the one or more nucleotide sequence encoding an anti-Plasmodium CSP antibody comprises one or more RNA sequence transcribed from one or more DNA sequences at least 95%, at least 96%, at least 97%, at least 98%, or at least 99% identical to one or more DNA sequences set forth in SEQ ID NO: 1, SEQ ID NO: 15, SEQ ID NO: 23, SEQ ID NO: 31, SEQ ID NO: 43, SEQ ID NO: 51, SEQ ID NO: 61, SEQ ID NO: 69, SEQ ID NO: 77, SEQ ID NO: 93, SEQ ID NO: 103, SEQ ID NO: 109, SEQ ID NO: 115, SEQ ID NO: 117, SEQ ID NO: 125, SEQ ID NO: 127, SEQ ID NO: 135, SEQ ID NO: 137, SEQ ID NO: 151, SEQ ID NO: 159, SEQ ID NO: 167, SEQ ID NO: 169, SEQ ID NO: 177, SEQ ID NO: 179, SEQ ID NO: 187, SEQ ID NO: 189, SEQ ID NO: 197, SEQ ID NO: 199, SEQ ID NO: 207, a variant thereof, or a fragment thereof.

In one embodiment, the one or more nucleotide sequence encoding an anti-Plasmodium CSP antibody comprises one or more RNA sequence transcribed from one or more DNA sequences as set forth in SEQ ID NO: 1, SEQ ID NO: 15, SEQ ID NO: 23, SEQ ID NO: 31, SEQ ID NO: 43, SEQ ID NO: 51, SEQ ID NO: 61, SEQ ID NO: 69, SEQ ID NO: 77, SEQ ID NO: 93, SEQ ID NO: 103, SEQ ID NO: 109, SEQ ID NO: 115, SEQ ID NO: 117, SEQ ID NO: 125, SEQ ID NO: 127, SEQ ID NO: 135, SEQ ID NO: 137, SEQ ID NO: 151, SEQ ID NO: 159, SEQ ID NO: 167, SEQ ID NO: 169, SEQ ID NO: 177, SEQ ID NO: 179, SEQ ID NO: 187, SEQ ID NO: 189, SEQ ID NO: 197, SEQ ID NO: 199, SEQ ID NO: 207, a variant thereof, or a fragment thereof.

In one embodiment, the one or more nucleotide sequence encoding an anti-Plasmodium CSP antibody comprises a nucleotide sequence encoding a heavy chain region, and a nucleotide sequence encoding a light chain region. In one embodiment, a single nucleic acid molecule comprises the nucleotide sequence encoding the heavy chain region, and the nucleotide sequence encoding the light chain region. In one embodiment, a first nucleic acid molecule comprises the nucleotide sequence encoding the heavy chain region, and a second nucleic acid molecule comprises the nucleotide sequence encoding the light chain region.

In one embodiment, the nucleotide sequence encoding the heavy chain region encodes an amino acid sequence at least 95%, at least 96%, at least 97%, at least 98%, or at least 99% identical to an amino acid sequence of SEQ ID NO: 4, SEQ ID NO: 18, SEQ ID NO: 26, SEQ ID NO: 34, SEQ ID NO: 46, SEQ ID NO: 54, SEQ ID NO: 64, SEQ ID NO: 72, SEQ ID NO: 80, SEQ ID NO: 96, SEQ ID NO: 112, SEQ ID NO: 120, SEQ ID NO: 130, SEQ ID NO: 140, SEQ ID NO: 162, SEQ ID NO: 172, SEQ ID NO: 182, SEQ ID NO: 192, or SEQ ID NO: 202. In one embodiment, the nucleotide sequence encoding the heavy chain region comprises a sequence encoding an amino acid sequence of SEQ ID NO: 4, SEQ ID NO: 18, SEQ ID NO: 26, SEQ ID NO: 34, SEQ ID NO: 46, SEQ ID NO: 54, SEQ ID NO: 64, SEQ ID NO: 72, SEQ ID NO: 80, SEQ ID NO: 96, SEQ ID NO: 112, SEQ ID NO: 120, SEQ ID NO: 130, SEQ ID NO: 140, SEQ ID NO: 162, SEQ ID NO: 172, SEQ ID NO: 182, SEQ ID NO: 192, or SEQ ID NO: 202.

In one embodiment, the nucleotide sequence encoding the heavy chain region comprises a nucleic acid sequence at least 95%, at least 96%, at least 97%, at least 98%, or at least 99% identical to a nucleic acid sequence of SEQ ID NO: 3, SEQ ID NO: 17, SEQ ID NO: 25, SEQ ID NO: 33, SEQ ID NO: 45, SEQ ID NO: 53, SEQ ID NO: 63, SEQ ID NO: 71, SEQ ID NO: 79, SEQ ID NO: 95, SEQ ID NO: 111, SEQ ID NO: 119, SEQ ID NO: 129, SEQ ID NO: 139, SEQ ID NO: 161, SEQ ID NO: 171, SEQ ID NO: 181, SEQ ID NO: 191, or SEQ ID NO: 201. In one embodiment, the nucleotide sequence encoding the heavy chain region comprises a nucleic acid sequence of SEQ ID NO: 3, SEQ ID NO: 17, SEQ ID NO: 25, SEQ ID NO: 33, SEQ ID NO: 45, SEQ ID NO: 53, SEQ ID NO: 63, SEQ ID NO: 71, SEQ ID NO: 79, SEQ ID NO: 95, SEQ ID NO: 111, SEQ ID NO: 119, SEQ ID NO: 129, SEQ ID NO: 139, SEQ ID NO: 161, SEQ ID NO: 171, SEQ ID NO: 181, SEQ ID NO: 191, or SEQ ID NO: 201.

In one embodiment, the nucleotide sequence encoding the light chain region encodes an amino acid sequence at least 95%, at least 96%, at least 97%, at least 98%, or at least 99% identical to an amino acid sequence of SEQ ID NO: 6, SEQ ID NO: 20, SEQ ID NO: 28, SEQ ID NO: 36, SEQ ID NO: 48, SEQ ID NO: 56, SEQ ID NO: 66, SEQ ID NO: 74, SEQ ID NO: 82, SEQ ID NO: 98, SEQ ID NO: 106, SEQ ID NO: 142, or SEQ ID NO: 154. In one embodiment, the nucleotide sequence encoding the light chain region comprises a sequence encoding an amino acid sequence of SEQ ID NO: 6, SEQ ID NO: 20, SEQ ID NO: 28, SEQ ID NO: 36, SEQ ID NO: 48, SEQ ID NO: 56, SEQ ID NO: 66, SEQ ID NO: 74, SEQ ID NO: 82, SEQ ID NO: 98, SEQ ID NO: 106, SEQ ID NO: 142, or SEQ ID NO: 154.

In one embodiment, the nucleotide sequence encoding the light chain region comprises a nucleic acid sequence at least 95%, at least 96%, at least 97%, at least 98%, or at least 99% identical to a nucleic acid sequence of SEQ ID NO: 5, SEQ ID NO: 19, SEQ ID NO: 27, SEQ ID NO: 35, SEQ ID NO: 47, SEQ ID NO: 55, SEQ ID NO: 65, SEQ ID NO: 73, SEQ ID NO: 81, SEQ ID NO: 97, SEQ ID NO: 105, SEQ ID NO: 141, or SEQ ID NO: 153. In one embodiment, the nucleotide sequence encoding the light chain region comprises a nucleic acid sequence of SEQ ID NO: 5, SEQ ID NO: 19, SEQ ID NO: 27, SEQ ID NO: 35, SEQ ID NO: 47, SEQ ID NO: 55, SEQ ID NO: 65, SEQ ID NO: 73, SEQ ID NO: 81, SEQ ID NO: 97, SEQ ID NO: 105, SEQ ID NO: 141, or SEQ ID NO: 153.

In one embodiment, the nucleotide sequence encoding the heavy chain region comprises nucleotide sequence encoding a variable heavy chain region and nucleotide sequence encoding a constant heavy chain region. In one embodiment, the nucleotide sequence encoding the light chain region comprises nucleotide sequence encoding a variable light chain region and a nucleotide sequence encoding a constant light chain region.

In one embodiment, the nucleotide sequence encoding the variable heavy chain region encodes an amino acid sequence at least 95%, at least 96%, at least 97%, at least 98%, or at least 99% identical to an amino acid sequence of SEQ ID NO: 8, SEQ ID NO: 22, SEQ ID NO: 38, SEQ ID NO: 58, SEQ ID NO: 76, SEQ ID NO: 100, SEQ ID NO: 114, SEQ ID NO: 122, SEQ ID NO: 132, SEQ ID NO: 144, SEQ ID NO: 164, SEQ ID NO: 174, SEQ ID NO: 184, SEQ ID NO: 194, or SEQ ID NO: 204. In one embodiment, the nucleotide sequence encoding the variable heavy chain region comprises a sequence encoding an amino acid sequence of SEQ ID NO: 8, SEQ ID NO: 22, SEQ ID NO: 38, SEQ ID NO: 58, SEQ ID NO: 76, SEQ ID NO: 100, SEQ ID NO: 114, SEQ ID NO: 122, SEQ ID NO: 132, SEQ ID NO: 144, SEQ ID NO: 164, SEQ ID NO: 174, SEQ ID NO: 184, SEQ ID NO: 194, or SEQ ID NO: 204.

In one embodiment, the nucleotide sequence encoding the variable heavy chain region comprises a nucleic acid sequence at least 95%, at least 96%, at least 97%, at least 98%, or at least 99% identical to a nucleic acid sequence of SEQ ID NO: 7, SEQ ID NO: 21, SEQ ID NO: 37, SEQ ID NO: 57, SEQ ID NO: 75, SEQ ID NO: 99, SEQ ID NO: 113, SEQ ID NO: 121, SEQ ID NO: 131, SEQ ID NO: 143, SEQ ID NO: 163, SEQ ID NO: 173, SEQ ID NO: 183, SEQ ID NO: 193, or SEQ ID NO: 203. In one embodiment, the nucleotide sequence encoding the variable heavy chain region comprises a nucleic acid sequence of SEQ ID NO: 7, SEQ ID NO: 21, SEQ ID NO: 37, SEQ ID NO: 57, SEQ ID NO: 75, SEQ ID NO: 99, SEQ ID NO: 113, SEQ ID NO: 121, SEQ ID NO: 131, SEQ ID NO: 143, SEQ ID NO: 163, SEQ ID NO: 173, SEQ ID NO: 183, SEQ ID NO: 193, or SEQ ID NO: 203.

In one embodiment, the nucleotide sequence encoding the variable light chain region encodes an amino acid sequence at least 95%, at least 96%, at least 97%, at least 98%, or at least 99% identical to an amino acid sequence of SEQ ID NO: 10, SEQ ID NO: 30, SEQ ID NO: 40, SEQ ID NO: 50, SEQ ID NO: 68, SEQ ID NO: 102, SEQ ID NO: 108, SEQ ID NO: 146, or SEQ ID NO: 156. In one embodiment, the nucleotide sequence encoding the variable light chain region comprises a sequence encoding an amino acid sequence of SEQ ID NO: 10, SEQ ID NO: 30, SEQ ID NO: 40, SEQ ID NO: 50, SEQ ID NO: 68, SEQ ID NO: 102, SEQ ID NO: 108, SEQ ID NO: 146, or SEQ ID NO: 156.

In one embodiment, the nucleotide sequence encoding the variable light chain region comprises a nucleic acid sequence at least 95%, at least 96%, at least 97%, at least 98%, or at least 99% identical to a nucleic acid sequence of SEQ ID NO: 9, SEQ ID NO: 29, SEQ ID NO: 39, SEQ ID NO: 49, SEQ ID NO: 67, SEQ ID NO: 101, SEQ ID NO: 107, SEQ ID NO: 145, or SEQ ID NO: 155. In one embodiment, the nucleotide sequence encoding the variable light chain region comprises a nucleic acid sequence of SEQ ID NO: 9, SEQ ID NO: 29, SEQ ID NO: 39, SEQ ID NO: 49, SEQ ID NO: 67, SEQ ID NO: 101, SEQ ID NO: 107, SEQ ID NO: 145, or SEQ ID NO: 155.

In one embodiment, the nucleotide sequence encoding the constant heavy chain region encodes an amino acid sequence at least 95%, at least 96%, at least 97%, at least 98%, or at least 99% identical to an amino acid sequence of SEQ ID NO: 12, SEQ ID NO: 124, SEQ ID NO: 134, SEQ ID NO: 148, SEQ ID NO: 166, SEQ ID NO: 176, SEQ ID NO: 186, SEQ ID NO: 196, or SEQ ID NO: 206. In one embodiment, the nucleotide sequence encoding the constant heavy chain region comprises a sequence encoding an amino acid sequence of SEQ ID NO: 12, SEQ ID NO: 124, SEQ ID NO: 134, SEQ ID NO: 148, SEQ ID NO: 166, SEQ ID NO: 176, SEQ ID NO: 186, SEQ ID NO: 196, or SEQ ID NO: 206.

In one embodiment, the nucleotide sequence encoding the constant heavy chain region comprises a nucleic acid sequence at least 95%, at least 96%, at least 97%, at least 98%, or at least 99% identical to a nucleic acid sequence of SEQ ID NO: 11, SEQ ID NO: 123, SEQ ID NO: 133, SEQ ID NO: 147, SEQ ID NO: 165, SEQ ID NO: 175, SEQ ID NO: 185, SEQ ID NO: 195, or SEQ ID NO: 205. In one embodiment, the nucleotide sequence encoding the constant heavy chain region comprises a nucleic acid sequence of SEQ ID NO: 11, SEQ ID NO: 123, SEQ ID NO: 133, SEQ ID NO: 147, SEQ ID NO: 165, SEQ ID NO: 175, SEQ ID NO: 185, SEQ ID NO: 195, or SEQ ID NO: 205.

In one embodiment, the nucleotide sequence encoding the constant light chain region encodes an amino acid sequence at least 95%, at least 96%, at least 97%, at least 98%, or at least 99% identical to an amino acid sequence of SEQ ID NO: 14, SEQ ID NO: 42, and SEQ ID NO: 60, SEQ ID NO: 150 or SEQ ID NO: 158. In one embodiment, the nucleotide sequence encoding the constant light chain region comprises a sequence encoding an amino acid sequence of SEQ ID NO: 14, SEQ ID NO: 42, SEQ ID NO: 60, SEQ ID NO: 150 or SEQ ID NO: 158.

In one embodiment, the nucleotide sequence encoding the constant light chain region comprises a nucleic acid sequence at least 95%, at least 96%, at least 97%, at least 98%, or at least 99% identical to a nucleic acid sequence of SEQ ID NO: 13, SEQ ID NO: 41, SEQ ID NO: 59, SEQ ID NO: 149, or SEQ ID NO: 157. In one embodiment, the nucleotide sequence encoding the constant light chain region comprises a nucleic acid sequence of SEQ ID NO: 13, SEQ ID NO: 41, SEQ ID NO: 59, SEQ ID NO: 149, or SEQ ID NO: 157.

The composition of the invention can treat, prevent and/or protect against any disease, disorder, or condition associated with Plasmodium infection. In certain embodiments, the composition can treat, prevent, and or/protect against parasitic infection. In certain embodiments, the composition can treat, prevent, and or/protect against a condition associated with Plasmodium infection.

The composition can result in the generation of the synthetic antibody in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the synthetic antibody in the subject within at least about 1 day, 2 days, 3 days, 4 days, 5 days, 6 days, 7 days, 8 days, 9 days, or 10 days of administration of the composition to the subject. The composition can result in generation of the synthetic antibody in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The composition, when administered to the subject in need thereof, can result in the generation of the synthetic antibody in the subject more quickly than the generation of an endogenous antibody in a subject who is administered an antigen to induce a humoral immune response. The composition can result in the generation of the synthetic antibody at least about 1 day, 2 days, 3 days, 4 days, 5 days, 6 days, 7 days, 8 days, 9 days, or 10 days before the generation of the endogenous antibody in the subject who was administered an antigen to induce a humoral immune response.

The composition of the present invention can have features required of effective compositions such as being safe so that the composition does not cause illness or death; being protective against illness; and providing ease of administration, few side effects, biological stability and low cost per dose.

### 3. RECOMBINANT NUCLEIC ACID SEQUENCE

As described above, the composition can comprise a recombinant nucleic acid sequence. The recombinant nucleic acid sequence can encode the antibody, a fragment thereof, a variant thereof, or a combination thereof. The antibody is described in more detail below.

The recombinant nucleic acid sequence can be a heterologous nucleic acid sequence. The recombinant nucleic acid sequence can include one or more heterologous nucleic acid sequences.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; addition of an internal IRES sequence and eliminating to the extent possible cis-acting sequence motifs (i.e., internal TATA boxes).

### Recombinant Nucleic Acid Sequence Construct

The recombinant nucleic acid sequence can include one or more recombinant nucleic acid sequence constructs. The recombinant nucleic acid sequence construct can include one or more components, which are described in more detail below.

The recombinant nucleic acid sequence construct can include a heterologous nucleic acid sequence that encodes a heavy chain polypeptide, a fragment thereof, a variant thereof, or a combination thereof. The recombinant nucleic acid sequence construct can include a heterologous nucleic acid sequence that encodes a light chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can also include a heterologous nucleic acid sequence that encodes an internal ribosome entry site (IRES). An IRES may be either a viral IRES or an eukaryotic IRES. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences. The tag sequence can encode a hemagglutinin (HA) tag.

### (1) Heavy Chain Polypeptide

The recombinant nucleic acid sequence construct can include the heterologous nucleic acid encoding the heavy chain polypeptide, a fragment thereof, a variant thereof, or a combination thereof. The heavy chain polypeptide can include a variable heavy chain (VH) region and/or at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

In some embodiments, the heavy chain polypeptide can include a VH region and a CH1 region. In other embodiments, the heavy chain polypeptide can include a VH region, a CH1 region, a hinge region, a CH2 region, and a CH3 region.

The heavy chain polypeptide can include a complementarity determining region (“CDR”) set. The CDR set can contain three hypervariable regions of the VH region. Proceeding from N-terminus of the heavy chain polypeptide, these CDRs are denoted “CDR1,” “CDR2,” and “CDR3,” respectively. CDR1, CDR2, and CDR3 of the heavy chain polypeptide can contribute to binding or recognition of the antigen.

### (2) Light Chain Polypeptide

The recombinant nucleic acid sequence construct can include the heterologous nucleic acid sequence encoding the light chain polypeptide, a fragment thereof, a variant thereof, or a combination thereof. The light chain polypeptide can include a variable light chain (VL) region and/or a constant light chain (CL) region.

The light chain polypeptide can include a complementarity determining region (“CDR”) set. The CDR set can contain three hypervariable regions of the VL region. Proceeding from N-terminus of the light chain polypeptide, these CDRs are denoted “CDR1,” “CDR2,” and “CDR3,” respectively. CDR1, CDR2, and CDR3 of the light chain polypeptide can contribute to binding or recognition of the antigen.

### (3) Protease Cleavage Site

The recombinant nucleic acid sequence construct can include heterologous nucleic acid sequence encoding a protease cleavage site. The protease cleavage site can be recognized by a protease or peptidase. The protease can be an endopeptidase or endoprotease, for example, but not limited to, furin, elastase, HtrA, calpain, trypsin, chymotrypsin, trypsin, and pepsin. The protease can be furin. In other embodiments, the protease can be a serine protease, a threonine protease, cysteine protease, aspartate protease, metalloprotease, glutamic acid protease, or any protease that cleaves an internal peptide bond (i.e., does not cleave the N-terminal or C-terminal peptide bond).

The protease cleavage site can include one or more amino acid sequences that promote or increase the efficiency of cleavage. The one or more amino acid sequences can promote or increase the efficiency of forming or generating discrete polypeptides. The one or more amino acids sequences can include a 2A peptide sequence.

### (4) Linker Sequence

The recombinant nucleic acid sequence construct can include one or more linker sequences. The linker sequence can spatially separate or link the one or more components described herein. In other embodiments, the linker sequence can encode an amino acid sequence that spatially separates or links two or more polypeptides.

### (5) Promoter

The recombinant nucleic acid sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleic acid sequence construct as it is from the transcription start site in its natural setting. However, variation in this distance may be accommodated without loss of promoter function.

The promoter may be operably linked to the heterologous nucleic acid sequence encoding the heavy chain polypeptide and/or light chain polypeptide. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metalothionein.

The promoter can be a constitutive promoter or an inducible promoter, which initiates transcription only when the host cell is exposed to some particular external stimulus. In the case of a multicellular organism, the promoter can also be specific to a particular tissue or organ or stage of development. The promoter may also be a tissue specific promoter, such as a muscle or skin specific promoter, natural or synthetic. Examples of such promoters are described in US patent application publication no. US20040175727, the contents of which are incorporated herein in its entirety.

The promoter can be associated with an enhancer. The enhancer can be located upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

### (6) Intron

The recombinant nucleic acid sequence construct can include one or more introns. Each intron can include functional splice donor and acceptor sites. The intron can include an enhancer of splicing. The intron can include one or more signals required for efficient splicing.

### (7) Transcription Termination Region

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

### (8) Initiation Codon

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

### (9) Termination Codon

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

### (10) Polyadenylation Signal

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA).

### (11) Leader Sequence

The recombinant nucleic acid sequence construct can include one or more leader sequences. The leader sequence can encode a signal peptide. The signal peptide can be an immunoglobulin (Ig) signal peptide, for example, but not limited to, an IgG signal peptide and a IgE signal peptide. In some embodiments, the signal peptide is encoded by one or more nucleic acid sequence selected from the group consisting of: SEQ ID NO: 87, SEQ ID NO: 89 and SEQ ID NO: 91. In some embodiments, the signal peptide comprises one or more amino acid sequence selected from the group consisting of: SEQ ID NO: 88, SEQ ID NO: 90 and SEQ ID NO: 92.

### Arrangement of the Recombinant Nucleic Acid Sequence Construct

As described above, the recombinant nucleic acid sequence can include one or more recombinant nucleic acid sequence constructs, in which each recombinant nucleic acid sequence construct can include one or more components. The one or more components are described in detail above. The one or more components, when included in the recombinant nucleic acid sequence construct, can be arranged in any order relative to one another. In some embodiments, the one or more components can be arranged in the recombinant nucleic acid sequence construct as described below.

### (12) Arrangement 1

In one arrangement, a first recombinant nucleic acid sequence construct can include the heterologous nucleic acid sequence encoding the heavy chain polypeptide and a second recombinant nucleic acid sequence construct can include the heterologous nucleic acid sequence encoding the light chain polypeptide. For example, in one embodiment, the first recombinant nucleic acid sequence encodes a heavy chain polypeptide having an amino acid sequence at least 95%, at least 96%, at least 97%, at least 98%, or at least 99% identical to an amino acid sequence of SEQ ID NO: 4, SEQ ID NO: 18, SEQ ID NO: 26, SEQ ID NO: 34, SEQ ID NO: 46, SEQ ID NO: 54, SEQ ID NO: 64, SEQ ID NO: 72, SEQ ID NO: 80, SEQ ID NO: 96, SEQ ID NO: 112, SEQ ID NO: 120, SEQ ID NO: 130, SEQ ID NO: 140, SEQ ID NO: 162, SEQ ID NO: 172, SEQ ID NO: 182, SEQ ID NO: 192, or SEQ ID NO: 202. In one embodiment, the first recombinant nucleic acid sequence comprises a nucleic acid sequence at least 95%, at least 96%, at least 97%, at least 98%, or at least 99% identical to a nucleic acid sequence of SEQ ID NO: 3, SEQ ID NO: 17, SEQ ID NO: 25, SEQ ID NO: 33, SEQ ID NO: 45, SEQ ID NO: 53, SEQ ID NO: 63, SEQ ID NO: 71, SEQ ID NO: 79, SEQ ID NO: 95, SEQ ID NO: 111, SEQ ID NO: 119, SEQ ID NO: 129, SEQ ID NO: 139, SEQ ID NO: 161, SEQ ID NO: 171, SEQ ID NO: 181, SEQ ID NO: 191, or SEQ ID NO: 201. In one embodiment, the second recombinant nucleic acid sequence encodes a light chain polypeptide having an amino acid sequence at least 95%, at least 96%, at least 97%, at least 98%, or at least 99% identical to an amino acid sequence of SEQ ID NO: 6, SEQ ID NO: 20, SEQ ID NO: 28, SEQ ID NO: 36, SEQ ID NO: 48, SEQ ID NO: 56, SEQ ID NO: 66, SEQ ID NO: 74, SEQ ID NO: 82, SEQ ID NO: 98, SEQ ID NO: 106, SEQ ID NO: 142, and SEQ ID NO: 154. In one embodiment, the second recombinant nucleic acid sequence comprises a nucleic acid sequence at least 95%, at least 96%, at least 97%, at least 98%, or at least 99% identical to a nucleic acid sequence of SEQ ID NO: 5, SEQ ID NO: 19, SEQ ID NO: 27, SEQ ID NO: 35, SEQ ID NO: 47, SEQ ID NO: 55, SEQ ID NO: 65, SEQ ID NO: 73, SEQ ID NO: 81, SEQ ID NO: 97, SEQ ID NO: 105, SEQ ID NO: 141, or SEQ ID NO: 153.

The first recombinant nucleic acid sequence construct can be placed in a vector. The second recombinant nucleic acid sequence construct can be placed in a second or separate vector. Placement of the recombinant nucleic acid sequence construct into the vector is described in more detail below.

The first recombinant nucleic acid sequence construct can also include the promoter, intron, transcription termination region, initiation codon, termination codon, and/or polyadenylation signal. The first recombinant nucleic acid sequence construct can further include the leader sequence, in which the leader sequence is located upstream (or 5′) of the heterologous nucleic acid sequence encoding the heavy chain polypeptide. Accordingly, the signal peptide encoded by the leader sequence can be linked by a peptide bond to the heavy chain polypeptide.

The second recombinant nucleic acid sequence construct can also include the promoter, initiation codon, termination codon, and polyadenylation signal. The second recombinant nucleic acid sequence construct can further include the leader sequence, in which the leader sequence is located upstream (or 5′) of the heterologous nucleic acid sequence encoding the light chain polypeptide. Accordingly, the signal peptide encoded by the leader sequence can be linked by a peptide bond to the light chain polypeptide.

### (13) Arrangement 2

In a second arrangement, the recombinant nucleic acid sequence construct can include the heterologous nucleic acid sequence encoding the heavy chain polypeptide and the heterologous nucleic acid sequence encoding the light chain polypeptide. The heterologous nucleic acid sequence encoding the heavy chain polypeptide can be positioned upstream (or 5′) of the heterologous nucleic acid sequence encoding the light chain polypeptide. Alternatively, the heterologous nucleic acid sequence encoding the light chain polypeptide can be positioned upstream (or 5′) of the heterologous nucleic acid sequence encoding the heavy chain polypeptide.

The recombinant nucleic acid sequence construct can be placed in the vector as described in more detail below.

The recombinant nucleic acid sequence construct can include a heterologous nucleic acid sequence encoding a cleavage site and/or a linker sequence. If included in the recombinant nucleic acid sequence construct, the heterologous nucleic acid sequence encoding the cleavage site can be positioned between the heterologous nucleic acid sequence encoding the heavy chain polypeptide and the heterologous nucleic acid sequence encoding the light chain polypeptide. Accordingly, the cleavage site allows for separation of the heavy chain polypeptide and the light chain polypeptide into distinct polypeptides upon expression. In other embodiments, if the linker sequence is included in the recombinant nucleic acid sequence construct, then the linker sequence can be positioned between the heterologous nucleic acid sequence encoding the heavy chain polypeptide and the heterologous nucleic acid sequence encoding the light chain polypeptide.

The recombinant nucleic acid sequence construct can also include the promoter, intron, transcription termination region, initiation codon, termination codon, and/or polyadenylation signal. The recombinant nucleic acid sequence construct can include one or more promoters. The recombinant nucleic acid sequence construct can include two promoters such that one promoter can be associated with the heterologous nucleic acid sequence encoding the heavy chain polypeptide and the second promoter can be associated with the heterologous nucleic acid sequence encoding the light chain polypeptide. In still other embodiments, the recombinant nucleic acid sequence construct can include one promoter that is associated with the heterologous nucleic acid sequence encoding the heavy chain polypeptide and the heterologous nucleic acid sequence encoding the light chain polypeptide.

The recombinant nucleic acid sequence construct can further include two leader sequences, in which a first leader sequence is located upstream (or 5′) of the heterologous nucleic acid sequence encoding the heavy chain polypeptide and a second leader sequence is located upstream (or 5′) of the heterologous nucleic acid sequence encoding the light chain polypeptide. Accordingly, a first signal peptide encoded by the first leader sequence can be linked by a peptide bond to the heavy chain polypeptide and a second signal peptide encoded by the second leader sequence can be linked by a peptide bond to the light chain polypeptide.

Expression from the Recombinant Nucleic Acid Sequence Construct

As described above, the recombinant nucleic acid sequence construct can include, amongst the one or more components, the heterologous nucleic acid sequence encoding the heavy chain polypeptide and/or the heterologous nucleic acid sequence encoding the light chain polypeptide. Accordingly, the recombinant nucleic acid sequence construct can facilitate expression of the heavy chain polypeptide and/or the light chain polypeptide.

When arrangement 1 as described above is utilized, the first recombinant nucleic acid sequence construct can facilitate the expression of the heavy chain polypeptide and the second recombinant nucleic acid sequence construct can facilitate expression of the light chain polypeptide. When arrangement 2 as described above is utilized, the recombinant nucleic acid sequence construct can facilitate the expression of the heavy chain polypeptide and the light chain polypeptide.

Upon expression, for example, but not limited to, in a cell, organism, or mammal, the heavy chain polypeptide and the light chain polypeptide can assemble into the synthetic antibody. In particular, the heavy chain polypeptide and the light chain polypeptide can interact with one another such that assembly results in the synthetic antibody being capable of binding the antigen. In other embodiments, the heavy chain polypeptide and the light chain polypeptide can interact with one another such that assembly results in the synthetic antibody being more immunogenic as compared to an antibody not assembled as described herein. In still other embodiments, the heavy chain polypeptide and the light chain polypeptide can interact with one another such that assembly results in the synthetic antibody being capable of eliciting or inducing an immune response against the antigen.

### Vector

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

Vectors include, but are not limited to, plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. In some embodiments, the vector includes linear DNA, enzymatic DNA or synthetic DNA. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleic acid sequence construct is produced by the cellular-transcription and translation machinery ribosomal complexes. The one or more vectors can express large amounts of stable messenger RNA, and therefore proteins.

### (14) Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

### (15) Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif.), which may be used for protein production in Escherichia coli (E. coli). The plasmid may also be pYES2 (Invitrogen, San Diego, Calif), which may be used for protein production in Saccharomyces cerevisiae strains of yeast. The plasmid may also be of the MAXBAC™ complete baculovirus expression system (Invitrogen, San Diego, Calif), which may be used for protein production in insect cells. The plasmid may also be pcDNAI or pcDNA3 (Invitrogen, San Diego, Calif.), which may be used for protein production in mammalian cells such as Chinese hamster ovary (CHO) cells.

### (16) RNA

In one embodiment, the nucleic acid is an RNA molecule. In one embodiment, the RNA molecule is transcribed from a DNA sequence described herein. For example, in some embodiments, the RNA molecule is encoded by a DNA sequence at least 90%, least 91%, least 92%, least 93%, least 94%, at least 95%, at least 96%, at least 97%, at least 98%, or at least 99% identical to one or more DNA sequence set forth in SEQ ID NO: 1, SEQ ID NO: 15, SEQ ID NO: 23, SEQ ID NO: 31, SEQ ID NO: 43, SEQ ID NO: 51, SEQ ID NO: 61, SEQ ID NO: 69, SEQ ID NO: 77, SEQ ID NO: 93, SEQ ID NO: 103, SEQ ID NO: 109, SEQ ID NO: 115, SEQ ID NO: 117, SEQ ID NO: 125, SEQ ID NO: 127, SEQ ID NO: 135, SEQ ID NO: 137, SEQ ID NO: 151, SEQ ID NO: 159, SEQ ID NO: 167, SEQ ID NO: 169, SEQ ID NO: 177, SEQ ID NO: 179, SEQ ID NO: 187, SEQ ID NO: 189, SEQ ID NO: 197, SEQ ID NO: 199, or SEQ ID NO: 207. In another embodiment, the nucleotide sequence comprises an RNA sequence transcribed by a DNA sequence encoding a polypeptide sequence of SEQ ID NO: 1, SEQ ID NO: 15, SEQ ID NO: 23, SEQ ID NO: 31, SEQ ID NO: 43, SEQ ID NO: 51, SEQ ID NO: 61, SEQ ID NO: 69, SEQ ID NO: 77, SEQ ID NO: 93, SEQ ID NO: 103, SEQ ID NO: 109, SEQ ID NO: 115, SEQ ID NO: 117, SEQ ID NO: 125, SEQ ID NO: 127, SEQ ID NO: 135, SEQ ID NO: 137, SEQ ID NO: 151, SEQ ID NO: 159, SEQ ID NO: 167, SEQ ID NO: 169, SEQ ID NO: 177, SEQ ID NO: 179, SEQ ID NO: 187, SEQ ID NO: 189, SEQ ID NO: 197, SEQ ID NO: 199, SEQ ID NO: 207, or a variant thereof or a fragment thereof. Accordingly, in one embodiment, the invention provides an RNA molecule encoding one or more of the DMAbs. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. A RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

### (17) Circular and Linear Vector

The one or more vectors may be circular plasmid, which may transform a target cell by integration into the cellular genome or exist extrachromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject via electroporation and expressing the heavy chain polypeptide and/or light chain polypeptide encoded by the recombinant nucleic acid sequence construct. The LEC may be any linear DNA devoid of any phosphate backbone. The LEC may not contain any antibiotic resistance genes and/or a phosphate backbone. The LEC may not contain other nucleic acid sequences unrelated to the desired gene expression.

The LEC may be derived from any plasmid capable of being linearized. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be pcrM2. The LEC can be pcrNP. pcrNP and pcrMR can be derived from pNP (Puerto Rico/34) and pM2 (New Caledonia/99), respectively.

### (18) Viral Vectors

In one embodiment, viral vectors are provided herein which are capable of delivering a nucleic acid of the invention to a cell. The expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

### (19) Method of Preparing the Vector

Provided herein is a method for preparing the one or more vectors in which the recombinant nucleic acid sequence construct has been placed. After the final subcloning step, the vector can be used to inoculate a cell culture in a large scale fermentation tank, using known methods in the art.

In other embodiments, after the final subcloning step, the vector can be used with one or more electroporation (EP) devices. The EP devices are described below in more detail.

The one or more vectors can be formulated or manufactured using a combination of known devices and techniques, but preferably they are manufactured using a plasmid manufacturing technique that is described in a licensed, co-pending U.S. provisional application U.S. Ser. No. 60/939,792, which was filed on May 23, 2007. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

### 4. ANTIBODY

As described above, the recombinant nucleic acid sequence can encode the antibody, a fragment thereof, a variant thereof, or a combination thereof. The antibody can bind or react with the antigen, which is described in more detail below.

The antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites. Accordingly, the antibody can be the Fab or F(ab′)₂. The Fab can include the heavy chain polypeptide and the light chain polypeptide. The heavy chain polypeptide of the Fab can include the VH region and the CH1 region. The light chain of the Fab can include the VL region and CL region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific antibody as described below in more detail. The antibody can be a bifunctional antibody as also described below in more detail.

As described above, the antibody can be generated in the subject upon administration of the composition to the subject. The antibody may have a half-life within the subject. In some embodiments, the antibody may be modified to extend or shorten its half-life within the subject. Such modifications are described below in more detail.

The antibody can be defucosylated as described in more detail below.

The antibody may be modified to reduce or prevent antibody-dependent enhancement (ADE) of disease associated with the antigen as described in more detail below.

### Bispecific Antibody

The recombinant nucleic acid sequence can encode a bispecific antibody, a fragment thereof, a variant thereof, or a combination thereof. The bispecific antibody can bind or react with two antigens, for example, two of the antigens described below in more detail. The bispecific antibody can be comprised of fragments of two of the antibodies described herein, thereby allowing the bispecific antibody to bind or react with two desired target molecules, which may include the antigen, which is described below in more detail, a ligand, including a ligand for a receptor, a receptor, including a ligand-binding site on the receptor, a ligand-receptor complex, and a marker.

The invention provides novel bispecific antibodies comprising a first antigen-binding site that specifically binds to a first target and a second antigen-binding site that specifically binds to a second target, with particularly advantageous properties such as producibility, stability, binding affinity, biological activity, specific targeting of certain T cells, targeting efficiency and reduced toxicity. In some instances, there are bispecific antibodies, wherein the bispecific antibody binds to the first target with high affinity and to the second target with low affinity. In other instances, there are bispecific antibodies, wherein the bispecific antibody binds to the first target with low affinity and to the second target with high affinity. In other instances, there are bispecific antibodies, wherein the bispecific antibody binds to the first target with a desired affinity and to the second target with a desired affinity.

In one embodiment, the bispecific antibody is a bivalent antibody comprising a) a first light chain and a first heavy chain of an antibody specifically binding to a first antigen, and b) a second light chain and a second heavy chain of an antibody specifically binding to a second antigen.

In some embodiments, one of the binding sites of an antibody molecule according to the invention is able to bind a T-cell specific receptor molecule and/or a natural killer cell (NK cell) specific receptor molecule. A T-cell specific receptor is the so called “T-cell receptor” (TCRs), which allows a T cell to bind to and, if additional signals are present, to be activated by and respond to an epitope/antigen presented by another cell called the antigen-presenting cell or APC. The T cell receptor is known to resemble a Fab fragment of a naturally occurring immunoglobulin. It is generally monovalent, encompassing. alpha.- and .beta.-chains, in some embodiments it encompasses .gamma.-chains and .delta.-chains (supra). Accordingly, in some embodiments the TCR is TCR (alpha/beta) and in some embodiments it is TCR (gamma/delta). The T cell receptor forms a complex with the CD3 T-Cell co-receptor. CD3 is a protein complex and is composed of four distinct chains. In mammals, the complex contains a CD3γ chain, a CD36 chain, and two CD3E chains. These chains associate with a molecule known as the T cell receptor (TCR) and the ζ-chain to generate an activation signal in T lymphocytes. Hence, in some embodiments a T-cell specific receptor is the CD3 T-Cell co-receptor. In some embodiments, a T-cell specific receptor is CD28, a protein that is also expressed on T cells. CD28 can provide co-stimulatory signals, which are required for T cell activation. CD28 plays important roles in T-cell proliferation and survival, cytokine production, and T-helper type-2 development. Yet a further example of a T-cell specific receptor is CD134, also termed Ox40. CD134/OX40 is being expressed after 24 to 72 hours following activation and can be taken to define a secondary costimulatory molecule. Another example of a T-cell receptor is 4-1 BB capable of binding to 4-1 BB-Ligand on antigen presenting cells (APCs), whereby a costimulatory signal for the T cell is generated. Another example of a receptor predominantly found on T-cells is CD5, which is also found on B cells at low levels. A further example of a receptor modifying T cell functions is CD95, also known as the Fas receptor, which mediates apoptotic signaling by Fas-ligand expressed on the surface of other cells. CD95 has been reported to modulate TCR/CD3-driven signaling pathways in resting T lymphocytes.

An example of a NK cell specific receptor molecule is CD16, a low affinity Fc receptor and NKG2D. An example of a receptor molecule that is present on the surface of both T cells and natural killer (NK) cells is CD2 and further members of the CD2-superfamily. CD2 is able to act as a co-stimulatory molecule on T and NK cells.

In some embodiments, the first binding site of the antibody molecule binds a Plasmodium parasite antigen and the second binding site binds a T cell specific receptor molecule and/or a natural killer (NK) cell specific receptor molecule. In some embodiments, the first binding site of the antibody molecule binds a Plasmodium CSP, and the second binding site binds a T cell specific receptor molecule and/or a natural killer (NK) cell specific receptor molecule. In some embodiments, the first binding site of the antibody molecule binds Plasmodium CSP and the second binding site binds one of CD3, the T cell receptor (TCR), CD28, CD16, NKG2D, Ox40, 4-1BB, CD2, CD5 and CD95.

In some embodiments, the first binding site of the antibody molecule binds a T cell specific receptor molecule and/or a natural killer (NK) cell specific receptor molecule and the second binding site binds a Plasmodium antigen. In some embodiments, the first binding site of the antibody binds a T cell specific receptor molecule and/or a natural killer (NK) cell specific receptor molecule and the second binding site binds Plasmodium CSP. In some embodiments, the first binding site of the antibody binds one of CD3, the T cell receptor (TCR), CD28, CD16, NKG2D, Ox40, 4-1BB, CD2, CD5 and CD95, and the second binding site binds Plasmodium CSP.

### Bifunctional Antibody

The recombinant nucleic acid sequence can encode a bifunctional antibody, a fragment thereof, a variant thereof, or a combination thereof. The bifunctional antibody can bind or react with the antigen described below. The bifunctional antibody can also be modified to impart an additional functionality to the antibody beyond recognition of and binding to the antigen. Such a modification can include, but is not limited to, coupling to factor H or a fragment thereof. Factor H is a soluble regulator of complement activation and thus, may contribute to an immune response via complement-mediated lysis (CML).

### Extension of Antibody Half-Life

As described above, the antibody may be modified to extend or shorten the half-life of the antibody in the subject. The modification may extend or shorten the half-life of the antibody in the serum of the subject.

The modification may be present in a constant region of the antibody. The modification may be one or more amino acid substitutions in a constant region of the antibody that extend the half-life of the antibody as compared to a half-life of an antibody not containing the one or more amino acid substitutions. The modification may be one or more amino acid substitutions in the CH2 domain of the antibody that extend the half-life of the antibody as compared to a half-life of an antibody not containing the one or more amino acid substitutions.

In some embodiments, the one or more amino acid substitutions in the constant region may include replacing a methionine residue in the constant region with a tyrosine residue, a serine residue in the constant region with a threonine residue, a threonine residue in the constant region with a glutamate residue, or any combination thereof, thereby extending the half-life of the antibody.

In other embodiments, the one or more amino acid substitutions in the constant region may include replacing a methionine residue in the CH2 domain with a tyrosine residue, a serine residue in the CH2 domain with a threonine residue, a threonine residue in the CH2 domain with a glutamate residue, or any combination thereof, thereby extending the half-life of the antibody.

### Defucosylation

The recombinant nucleic acid sequence can encode an antibody that is not fucosylated (i.e., a defucosylated antibody or a non-fucosylated antibody), a fragment thereof, a variant thereof, or a combination thereof. Fucosylation includes the addition of the sugar fucose to a molecule, for example, the attachment of fucose to N-glycans, O-glycans and glycolipids. Accordingly, in a defucosylated antibody, fucose is not attached to the carbohydrate chains of the constant region. In turn, this lack of fucosylation may improve FcγRIIIa binding and antibody directed cellular cytotoxic (ADCC) activity by the antibody as compared to the fucosylated antibody. Therefore, in some embodiments, the non-fucosylated antibody may exhibit increased ADCC activity as compared to the fucosylated antibody.

The antibody may be modified so as to prevent or inhibit fucosylation of the antibody. In some embodiments, such a modified antibody may exhibit increased ADCC activity as compared to the unmodified antibody. The modification may be in the heavy chain, light chain, or a combination thereof. The modification may be one or more amino acid substitutions in the heavy chain, one or more amino acid substitutions in the light chain, or a combination thereof.

### Reduced ADE Response

The antibody may be modified to reduce or prevent antibody-dependent enhancement (ADE) of disease associated with the antigen, but still neutralize the antigen.

In some embodiments, the antibody may be modified to include one or more amino acid substitutions that reduce or prevent binding of the antibody to FcγR1a. The one or more amino acid substitutions may be in the constant region of the antibody. The one or more amino acid substitutions may include replacing a leucine residue with an alanine residue in the constant region of the antibody, i.e., also known herein as LA, LA mutation or LA substitution. The one or more amino acid substitutions may include replacing two leucine residues, each with an alanine residue, in the constant region of the antibody and also known herein as LALA, LALA mutation, or LALA substitution. The presence of the LALA substitutions may prevent or block the antibody from binding to FcγR1a, and thus, the modified antibody does not enhance or cause ADE of disease associated with the antigen, but still neutralizes the antigen.

### 5. ANTIGEN

The synthetic antibody is directed to the antigen or fragment or variant thereof. The antigen can be a nucleic acid sequence, an amino acid sequence, a polysaccharide or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The polysaccharide can be a nucleic acid encoded polysaccharide.

The antigen can be from a parasite. The antigen can be associated with parasitic infection. In one embodiment, the antigen can be associated with Plasmodium infection. In one embodiment, the antigen can be a Plasmodium circumsporozoite protein (CSP).

In one embodiment, a synthetic antibody of the invention targets two or more antigens. In one embodiment, at least one antigen of a bispecific antibody is selected from the antigens described herein. In one embodiment, the two or more antigens are selected from the antigens described herein.

### Parasitic Antigens

The parasitic antigen can be a parasite antigen or fragment or variant thereof. The parasite can be a disease causing parasite. The parasite can be a Plasmodium parasite.

The antigen may be a Plasmodium parasitic antigen, or fragment thereof, or variant thereof. The Plasmodium antigen can be from a factor that allows the parasite to replicate, infect or survive. In one embodiment, a Plasmodium parasitic antigen is Plasmodium CSP.

The asymptomatic sporozoite stage occurs after parasite delivery and before liver invasion. The predominant surface protein of the sporozoite is the circumsporozoite protein (CSP). CSP is associated with infectivity of sporozoites and contains both B and T cell epitopes.

### 6. EXCIPIENTS AND OTHER COMPONENTS OF THE COMPOSITION

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition may further comprise a genetic facilitator agent as described in U.S. Ser. No. 021,579 filed Apr. 1, 1994, which is fully incorporated by reference.

The composition may comprise DNA at quantities of from about 1 nanogram to 100 milligrams; about 1 microgram to about 10 milligrams; or preferably about 0.1 microgram to about 10 milligrams; or more preferably about 1 milligram to about 2 milligram. In some preferred embodiments, composition according to the present invention comprises about 5 nanogram to about 1000 micrograms of DNA. In some preferred embodiments, composition can contain about 10 nanograms to about 800 micrograms of DNA. In some preferred embodiments, the composition can contain about 0.1 to about 500 micrograms of DNA. In some preferred embodiments, the composition can contain about 1 to about 350 micrograms of DNA. In some preferred embodiments, the composition can contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of DNA.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

### 7. METHOD OF GENERATING THE SYNTHETIC ANTIBODY

The present invention also relates a method of generating the synthetic antibody. The method can include administering the composition to the subject in need thereof by using the method of delivery described in more detail below. Accordingly, the synthetic antibody is generated in the subject or in vivo upon administration of the composition to the subject.

The method can also include introducing the composition into one or more cells, and therefore, the synthetic antibody can be generated or produced in the one or more cells. The method can further include introducing the composition into one or more tissues, for example, but not limited to, skin and muscle, and therefore, the synthetic antibody can be generated or produced in the one or more tissues.

### 8. METHOD OF IDENTIFYING OR SCREENING FOR THE ANTIBODY

The present invention further relates to a method of identifying or screening for the antibody described above, which is reactive to or binds the antigen described above. The method of identifying or screening for the antibody can use the antigen in methodologies known in those skilled in art to identify or screen for the antibody. Such methodologies can include, but are not limited to, selection of the antibody from a library (e.g., phage display) and immunization of an animal followed by isolation and/or purification of the antibody.

### 9. METHOD OF DELIVERY OF THE COMPOSITION

The present invention also relates to a method of delivering the composition to the subject in need thereof. The method of delivery can include, administering the composition to the subject. Administration can include, but is not limited to, DNA injection with and without in vivo electroporation, liposome mediated delivery, and nanoparticle facilitated delivery.

The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

### Electroporation

Administration of the composition via electroporation may be accomplished using electroporation devices that can be configured to deliver to a desired tissue of a mammal, a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA EP system (Inovio Pharmaceuticals, Plymouth Meeting, PA) or Elgen electroporator (Inovio Pharmaceuticals, Plymouth Meeting, PA) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the composition of the present invention, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the composition include those provided in co-pending and co-owned U.S. patent application Ser. No. 11/874,072, filed Oct. 17, 2007, which claims the benefit under 35 USC 119(e) to U.S. Provisional Applications Ser. No. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference.

The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to method of injecting DNA are contemplated herein. The above-patents are incorporated by reference in their entirety.

### 10. METHOD OF TREATMENT

Also provided herein is a method of treating, protecting against, and/or preventing disease in a subject in need thereof by generating the synthetic antibody in the subject. The method can include administering the composition to the subject. Administration of the composition to the subject can be done using the method of delivery described above.

In certain embodiments, the invention provides a method of treating protecting against, and/or preventing a Plasmodium parasitic infection. In one embodiment, the method treats, protects against, and/or prevents a disease associated with Plasmodium parasite infection. In one embodiment, the Plasmodium parasite includes, but it is not limited to, Plasmodium falciparum, Plasmodium vivax, Plasmodium ovale, Plasmodium malariae, and Plasmodium knowlesi.

Upon generation of the synthetic antibody in the subject, the synthetic antibody can bind to or react with the antigen. Such binding can neutralize the antigen, block recognition of the antigen by another molecule, for example, a protein or nucleic acid, and elicit or induce an immune response to the antigen, thereby treating, protecting against, and/or preventing the disease associated with the antigen in the subject.

The synthetic antibody can treat, prevent, and/or protect against disease in the subject administered the composition. The synthetic antibody by binding the antigen can treat, prevent, and/or protect against disease in the subject administered the composition. The synthetic antibody can promote survival of subject administered the composition. In one embodiment, the synthetic antibody can provide increased survival of the subject over the expected survival of a subject having the disease who has not been administered the synthetic antibody. In various embodiments, the synthetic antibody can provide at least about a 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or a 100% increase in survival of subjects administered the composition over the expected survival in the absence of the composition. In one embodiment, the synthetic antibody can provide increased protection against the disease in the subject over the expected protection of a subject who has not been administered the synthetic antibody. In various embodiments, the synthetic antibody can protect against disease in at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% of subjects administered the composition over the expected protection in the absence of the composition.

The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

### 11. USE IN COMBINATION

The present invention also provides a method of treating, protecting against, and/or preventing disease in a subject in need thereof by administering a combination of the synthetic antibody and a therapeutic agent. In one embodiment, the therapeutic agent is an antiparasitic agent.

The synthetic antibody and a therapeutic agent may be administered using any suitable method such that a combination of the synthetic antibody and therapeutic agent are both present in the subject. In one embodiment, the method may comprise administration of a first composition comprising a synthetic antibody of the invention by any of the methods described in detail above and administration of a second composition comprising a therapeutic agent less than 1, less than 2, less than 3, less than 4, less than 5, less than 6, less than 7, less than 8, less than 9 or less than 10 days following administration of the synthetic antibody. In one embodiment, the method may comprise administration of a first composition comprising a synthetic antibody of the invention by any of the methods described in detail above and administration of a second composition comprising a therapeutic agent more than 1, more than 2, more than 3, more than 4, more than 5, more than 6, more than 7, more than 8, more than 9 or more than 10 days following administration of the synthetic antibody. In one embodiment, the method may comprise administration of a first composition comprising a therapeutic agent and administration of a second composition comprising a synthetic antibody of the invention by any of the methods described in detail above less than 1, less than 2, less than 3, less than 4, less than 5, less than 6, less than 7, less than 8, less than 9 or less than 10 days following administration of the therapeutic agent. In one embodiment, the method may comprise administration of a first composition comprising a therapeutic agent and administration of a second composition comprising a synthetic antibody of the invention by any of the methods described in detail above more than 1, more than 2, more than 3, more than 4, more than 5, more than 6, more than 7, more than 8, more than 9 or more than 10 days following administration of the therapeutic agent. In one embodiment, the method may comprise administration of a first composition comprising a synthetic antibody of the invention by any of the methods described in detail above and a second composition comprising a therapeutic agent concurrently. In one embodiment, the method may comprise administration of a first composition comprising a synthetic antibody of the invention by any of the methods described in detail above and a second composition comprising a therapeutic agent concurrently. In one embodiment, the method may comprise administration of a single composition comprising a synthetic antibody of the invention and a therapeutic agent.

Non-limiting examples of antiparasitic therapeutic agents that can be used in combination with the synthetic antibody of the invention to protect against Plasmodium infection include Atovaquone, Proguanil, Chloroquine, Doxycycline, Mefloquine, Primaquine, and Tafenoquine. Non-limiting examples of antiparasitic therapeutic agents that can be used in combination with the synthetic antibody of the invention to treat Plasmodium infection include Artemether, Lumefantrine, Atovaquone, Proguanil, Quinine sulfate, doxycycline, tetracycline, clindamycin, Mefloquine, Chloroquine phosphate, Hydroxychloroquine, and Primaquine phosphate.

### 12. GENERATION OF SYNTHETIC ANTIBODIES IN VITRO AND EX VIVO

In one embodiment, the synthetic antibody is generated in vitro or ex vivo. For example, in one embodiment, a nucleic acid encoding a synthetic antibody can be introduced and expressed in an in vitro or ex vivo cell. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

### 13. EXAMPLES

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

There have been numerous attempts to develop malaria vaccines or therapeutics targeting different aspects of the parasite life cycle (Mahmoudi and Keshavarz, 2017). The asymptomatic sporozoite stage occurs after parasite delivery and before liver invasion. The predominant surface protein of the sporozoite is the circumsporozoite protein (CSP). CSP is associated with infectivity of sporozoites and contains both B and T cell epitopes (Florens et al., 2002, Ménard et al., 1997, Aldrich et al., 2012, Zavala et al., 1985). RTS,S/ASO1 is an advanced malaria vaccine candidate that has recently been introduced in a clinical setting in several African countries. Phase III trial results demonstrated that RTS,S, which contains portions of the central repeat domain and C terminal T cell epitopes of Plasmodium falciparum: CSP (PfCSP) scaffolded to the Hepatitis B virus surface antigen (HBsAg), induced a reduction in malaria cases in children over a span of 5 years (Stoute et al., 1997, RTS S Clinical Trials Partnership, 2015, Mahmoudi and Keshavarz, 2017). Another vaccine developed by Sanaria, PfSPZ, contains attenuated whole sporozoites. One distinct advantage of PfSPZ is that it presents PfCSP in its native conformation; PfSPZ has demonstrated protection in a Phase 1 trial (Hoffman et al., 2010, Seder et al., 2013). Correlates of protection from irradiated sporozoites include both antibody and T cell responses (Sissoko et al., 2017, Roestenberg et al., 2020). However, challenges with sporozoite vaccine models persist, as sporozoites may not be effective at priming immune responses if over-irradiated or an active infection may ensue if under-irradiated (Goh et al., 2019), supporting that additional tools may be important.

Monoclonal antibodies (mAbs) have also been isolated from malaria vaccinees and have defined new sites of vulnerability on the CSP antigen. MGU10 is a human IgG3 mAb isolated from a volunteer vaccinated with the PfSPZ vaccine. MGU10 exhibits dual specificity to PfCSP through binding to both the NANP repeat region as well as the junction between the N terminus and central repeat region. MGU10 demonstrated potent reduction in liver burden in mice (Tan et al., 2018b). CIS43 is a human IgG1 mAb isolated from a volunteer immunized with the PfSPZ vaccine. This mAb, like MGU10, exhibits preferential binding to a distinct epitope spanning the N terminus and NANP repeat junction of PfCSP as well as binding to the classically immunodominant NANP repeat region. Passive transfer of CIS43 also demonstrated protection in murine models of malaria (Kisalu et al., 2018).

Recombinant mAbs for infectious disease prophylaxis/therapy are becoming more common, yet there are distinct technological limitations including the high cost of production and the short half-life of the molecules in vivo (Patel et al., 2020, Kelley, 2009). DNA-encoded monoclonal antibodies (DMAbs) are directly launched in vivo and result in systemic, prolonged circulation of intact antibodies (Patel et al., 2020). Several DMAbs have been engineered and advanced, targeting a variety of important immune targets, which can impact outcomes in various disease models (Patel et al., 2017, Patel et al., 2018, Elliott et al., 2017, Wise et al., 2020, Flingai et al., 2015, Muthumani et al., 2016).

Herein, the design and development of anti-PfCSP DMAbs based on recombinant pre-erythrocytic mAbs CIS43 and MGU10 is described and they were evaluated in vitro, in vivo, and in malaria challenge models. Following optimization studies and down selection of specific constructs encoding CIS43 and MGU10-based DMAbs, it was observed that 43e DMAb-4 was the most potent in vivo and was subsequently advanced to study its ability to impact infection in a mosquito bite challenge model. Importantly, 43e DMAb-4 exhibited similar inhibition of liver infection as the positive control monoclonal antibody, mAb 311 (Oyen et al., 2017). In addition, 60% of mice receiving 43e DMAb-4 developed sterilizing immunity to Plasmodium infection.

The materials and methods of the present Example are now herein described.

### DMAb Construction and Plasmid Synthesis

The sequences used for all DMAbs were derived from the sequences for anti-PfCSP monoclonal antibodies CIS43 and MGU10 (Kisalu et al., 2018, Tan et al., 2018b). Exemplary nucleotide and constituent amino acid sequences were obtained from patents (CIS43 US20190375831A1; MGU10 US20200093909A1). The nucleotide sequences for both heavy and light chain Fab and Fc regions of each clone were codon-optimized for mouse and human to enhance transgene expression. In addition, N terminal framework modifications were introduced in both heavy and light chains. Based on an analysis using the IGMT DomainGapAlign Tool, select amino acid residues in the framework region were reverted back to the germline immunoglobulin gene sequence of highest similarity (Ehrenmann et al., 2009, Ehrenmann and Lefranc, 2011, Scaviner et al., 1999). The optimized HC and LC sequences were inserted into a single plasmid or dual plasmid system with HC and LC encoded on separate plasmids. In either case, a pVAX plasmid DNA expression vector was used. The genes were under the control of a human cytomegalovirus promoter as well as a bovine growth hormone polyA. For single plasmid constructs, the HC and LC genes were encoded in cis, with a furin cleavage site and porcine teschovirus-1 2A (P2A) peptide separating the two. For dual plasmid constructs, the HC and LC were encoded separately into pVAX expression vectors. The CIS43 molecular model was adapted from PDB 6B5M and MGU10 wild type amino acid sequence was input into Lymphocyte Receptor Automated Modeling (LYRA) (Klausen et al., 2015). PyMOL Molecular Graphics System V2.4.1 was used to label and generate 3D model.

### Cell Lines and Transfection

Expi293F transfection kit (Thermo Fisher Scientific, Waltham, MA) was used for all transfections. Protocol was followed per manufacturers specifications. Briefly, Expi293F cells were maintained in Expi293 Expression Medium. Cells were incubated on an orbital shaker at 37° C. in 8% CO₂ conditions. For transfection, 2×10⁶ Expi293F cells were plated in 2 mL Expression Medium per well above 95% viability. DNA plasmids were added to Opti-MEM media separately from ExpiFectamine transfection reagent. After a 5 minute incubation, the DNA and ExpiFectamine were combined for 20 minutes to allow for complexation. The DNA plasmid complex was then added to Expi293F cells. After 18 hours, Transfection Enhancers were added. Three days after the addition of Transfection Enhancers, cell supernatants were collected for further experimentation.

### Western Blot

For detection of antibody in transfected cell supernatants, approximately 50 μg of total supernatant protein was run on NuPAGE™ 4-12% Bis-Tris gels (ThermoFisher) in 1× NuPAGE™ MOPS buffer. Samples were prepared using NuPAGE™ LDS sample buffer and deionized water. If samples were reduced, NuPAGE™ Sample Reducing Agent was added. Samples were heated at 70° C. for 10 minutes in before being loaded onto the gel. The contents of the gel were transferred to a methanol-activated PVDF membrane via iBlot 2 Dry Blotting System (Life Technologies, Carlsbad, CA, USA). Upon transfer completion, the PVDF membrane was blocking with Odyssey Blocking Buffer (LI-COR) for one hour at room temperature (RT). After blocking was completed, the membrane was incubated with a murine anti-0 actin monoclonal antibody overnight at 4° C. The following day, the membrane was washed with PBS+0.1% Tween-20 three times followed by PBS alone. The membrane was then incubated with goat anti-mouse and goat anti-human IRDye labelled secondary antibody (LI-COR) in Intercept Blocking Buffer (LI-COR), 10% SDS (ThermoFisher), and Tween-20 for one hour at RT. Following a second series of washes, the membrane was imaged using LI-COR Odyssey CLx.

### Quantification ELISA

For quantification of transfection supernatants and sera, ThermoFisher MaxiSorp 96-well plates (Thermo Fisher Scientific, Waltham, MA) were coated with 5 μg mL⁻¹ goat anti-human IgG-Fc (Bethyl Laboratories, Montgomery, TX) overnight at 4° C. The following day, each plate was washed with PBS (Corning Inc., Corning, NY)+0.01% Tween-20 (ThermoFisher, Waltham, MA) (PBS-T) four times (4×). Plates were then blocked with 5% milk in PBS for 2 hours at RT. Upon completion of blocking, plates were washed again 4× with PBS-T and samples diluted in 1% NCS in PBS-T were transferred to plates for a 1 hour incubation at RT. A standard curve was generated using purified human IgGx or IgG, (Bethyl Laboratories, Montgomery, TX). Plates were subsequently washed and goat anti-human IgGx or IgGk HRP-conjugated secondary antibody (Bethyl Laboratories) was diluted to 1:10,000 and transferred onto plates for 1 hour at RT. After secondary incubation, plates were washed 4× and developed using OPD Substrate Tablets (Thermo Scientific) for 10 minutes in the dark and stopped with 2N H₂SO₄. The Biotek Synergy 2 plate reader was used to read absorbance at 450 nm.

### Binding ELISA

For binding detection of transfection supernatants and sera, the ELISA protocol is as described above. However, recombinant CSP (rCSP) at a concentration of 1 μg mL⁻¹ was used as coat protein. The secondary antibody used was HRP-conjugated goat anti-human Fe (Bethyl Laboratories). In addition, 1-Step™ Ultra TMB (Thermo Scientific) was used as detection substrate and allowed to incubate for 5 minutes before quenching with 2N H₂SO₄. Finally, the sera incubation occurred for 1 hr at 37° C. rather than at RT. The Biotek Synergy 2 plate reader was used to read absorbance at 450 nm.

### Sporozoite ELISA

For binding detection of sera to sporozoites, P. falciparum sporozoite-coated plates were prepared as described (Zavala et al., 1983). Briefly, plates were kept at −80° C. until time of assay. Plates were allowed to thaw at room temperature, media was removed and plates were washed 4× with PBS. Plates were blocked, washed, incubated, and developed as shown in Binding ELISA protocol above.

### Animal Experiments and Immunizations

Female BALB/cJ mice at 6 to 8 weeks of age were purchased from the Jackson Laboratory. Animal experiments were conducted under protocol #201236 approved by the Wistar Institute Institutional Animal Care and Use Committee (IACUC). All animals were housed in the Wistar Institute Animal Facility. For the in vivo expression experiments, mice were immunized with DNA plasmids intramuscularly (IM) at four sites: the left and right tibialis anterior (TA) muscle and left and right quadriceps. A total of 100 μg or 200 μg of DMAb plasmid was administered depending on the study, with a maximum DNA dose of 100 μg per leg. DNA mixed in sterile water was co-formulated with hyaluronidase (200 U/L, Sigma Aldrich, Saint Louis, MO) at a 1:1 ratio. A total of 30 μL was injected IM at each site. After IM injection, mice were electroporated at each injection site using the CELLECTRA 3P adaptive electroporation device (Inovio Pharmaceuticals, Bluebell, PA). For studies with CD4+ and CD8+ T cell depletion, 200 μg each of anti-CD4 (Bio X Cell clone GK1.5) and anti-CD8 (Bio X Cell clone YTS169.4) mAbs were administered intraperitoneally 5 minutes prior to DMAb administration to avoid an anti-human antibody response as we have described (Patel et al., 2017, Patel et al., 2018).

### Mosquito Bite Challenge Model

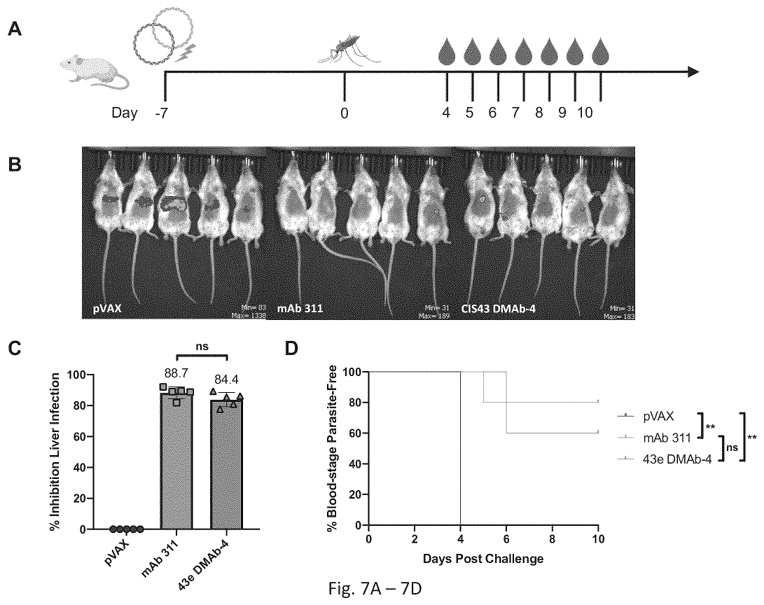

Seven days prior to challenge, mice were administered 200 μg of 43e DMAb-4 or 25 μg of pVAX empty vector negative control. Sixteen hours prior to challenge, the positive control group received 100 μg of mAb 311 intraperitoneally. On the day of challenge, mice were challenged as described (Flores-Garcia et al., 2019) by exposure to the bites of 5 Anopheles stephensi mosquitos infected with transgenic Plasmodium berghei sporozoites expressing full-length Plasmodium falciparum: CSP, as well as a luciferase reporter to visually quantify liver parasite burden (PbPfLuc). Following challenge, mosquitos were evaluated for being positive for blood meal. Liver parasite burden was examined 42 hours post mosquito bite challenge using IVIS Spectrum In Vivo Imaging System (PerkinElmer). Mice were intraperitoneally administered 100 μL of D-Luciferin at a concentration of 30 mg/mL prior to isoflurane anesthetization and imaging using IVIS Spectrum. Readout was bioluminescence expressed by the PbPfLuc parasites as measured in photons/sec. Beginning at four and proceeding daily until ten days post challenge, blood smears were conducted to determine the appearance of blood-stage infection (FIG. 7A).

### Statistical Analyses

All statistical computations were performed using PRISM v8.4.3. To achieve statistical significance in animal expression and challenge studies, n=5 mice/group was used. Kaplan-Meier survival curves were used to present percent blood-stage parasitemia-free mice in challenged groups. We performed ordinary one-way analysis of variance (ANOVA) to determine statistical significance between groups of three or more (Tukey's multiple comparison test) where necessary, as well as Mann Whitney Tests where treatment groups were compared to negative control. Percent inhibition of liver infection was calculated by dividing the geometric mean bioluminescence of a treatment group by the geometric mean bioluminescence of the pVAX negative control and subtracting the result from 1. Survival curves were analyzed using Log-rank (Mantel Cox) test. For these data to be considered statistically significant, p<0.05.

The Experimental results are now herein described.

### Engineered 10e DMAbs Express and Bind Recombinant CSP Both In Vitro and In Vivo

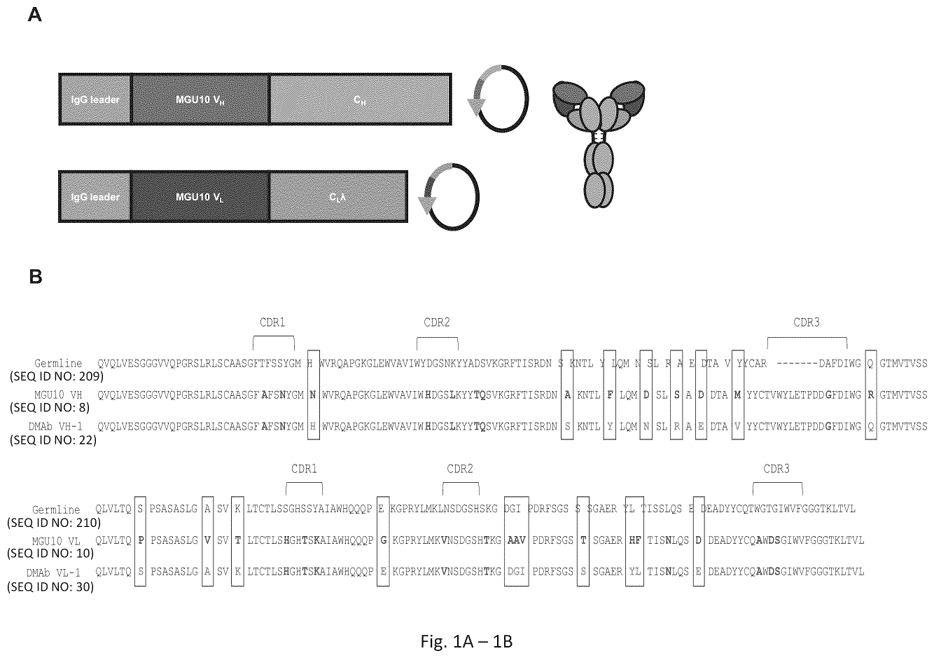

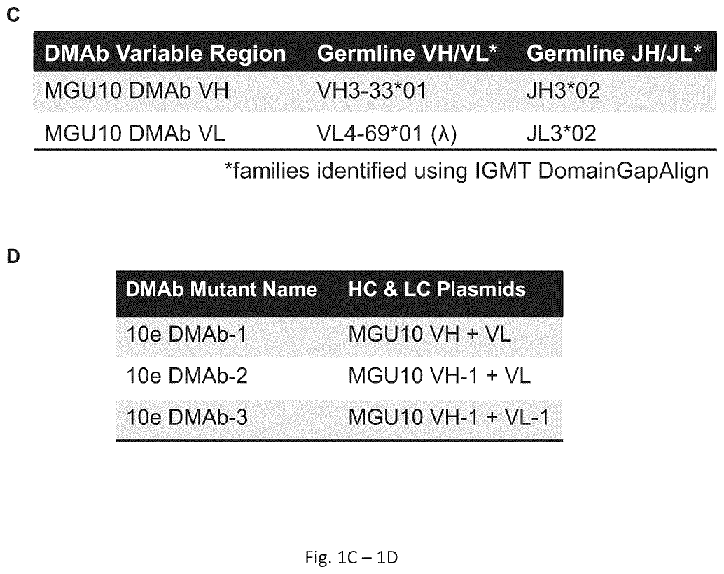

MGU10, a monoclonal antibody with dual specificity for both the NANP repeats as well as the junction between the N terminus and NPNA repeats of CSP, was chosen as a clone for first generation DMAb engineering and synthesis (Tan et al., 2018b). A dual plasmid approach where heavy and light chains are encoded onto separate plasmids was used (FIG. 1A). It has been observed that encoding DMAbs on two plasmids can improve in vivo expression (Elliott et al., 2017, Patel et al., 2018). Variable regions were cloned in after a human IgG leader sequence and preceding a human IgG1 constant region to extend antibody half-life (Mankarious et al., 1988).

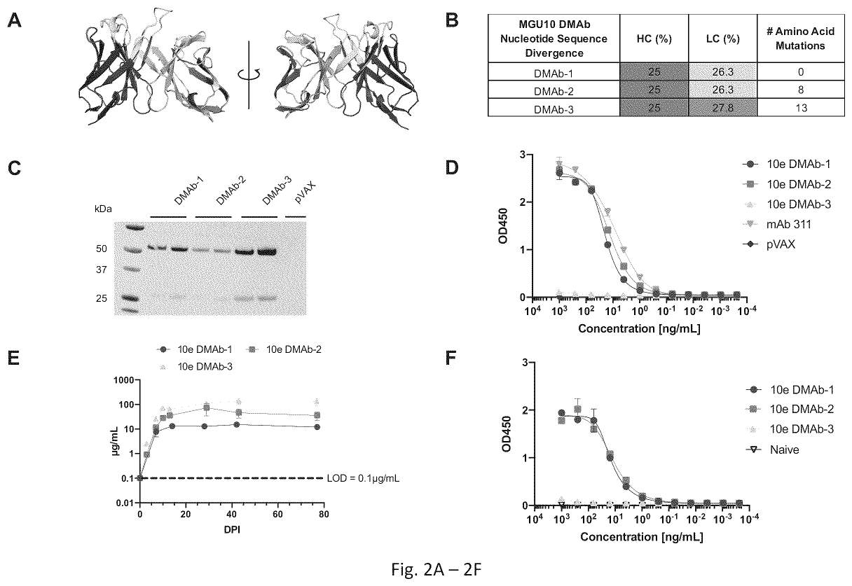

In total, three MGU10-based engineered DMAb (10e DMAb) constructs were designed: one retained the original clone amino acid sequence, while the other two contained point mutations where amino acid residues were selectively reverted to their germline configuration (FIG. 1B). It has been observed that reverting specific residues in the framework region away from the hypervariable regions back to their germline configuration can be an important tool to improve DMAb expression (Patel et al., 2018). Sequences were analyzed using IMGT DomainGapAlign tool (Ehrenmann et al., 2009, Ehrenmann and Lefranc, 2011, Scaviner et al., 1999) to identify the immunoglobulin gene family as well as sites for potential modification (Ehrenmann et al., 2009, Ehrenmann and Lefranc, 2011, Scaviner et al., 1999). Sites throughout the framework region were selectively mutated; a graphical representation based on predictive modeling using LYRA shows all potential sites of mutation, and each DMAb construct has a subset of these changes (FIG. 2A) (Klausen et al., 2015). A table illustrating the germline genes associated with the clone that were used for germline engineering can be found in FIG. 1C-D. In addition to amino acid changes, codon optimization for expression in murine and human systems was performed, changing the identity of various nucleotides in 10e DMAbs compared to heavy and light chain variable regions of WT MGU10 (FIG. 2B). Heavy chain variable region nucleotide divergence was 25% for all clones, but light chain variable region nucleotide divergence varied from 26.3% to 27.8%. However, on the amino acid level, the DMAbs range from 0% to 5.6% divergent, with an average variable region amino acid divergence of 3%.

To probe for in vitro expression of the 10e DMAb constructs, Expi293F cells were transfected with synthetic DNA plasmids encoding HC and LC of each DMAb construct. Supernatants were collected for a subsequent analysis by Western Blot and binding ELISA. All three 10e DMAb constructs expressed in vitro with heavy and light chain bands occurring at approximately 50 and 25 kDa respectively (FIG. 2C). In addition, supernatants containing 10e DMAb-1 and DMAb-2 constructs potently bound recombinant CSP (rCSP), whereas 10e DMAb-3 exhibited low binding (FIG. 2D). Supernatants containing 10e DMAb-1 and 2 exhibited binding to rCSP similar to that of mAb 311, a recombinant monoclonal antibody targeting the NANP repeats of CSP (FIG. 2D) (Oyen et al., 2018). After the preliminary in vitro screening determined the expression and binding profile of the 10e DMAbs, each construct was evaluated in vivo. Sera isolated from DMAb-administered mice (n=5 mice/group) demonstrated expression of each 10e DMAb construct with peak concentration in mouse sera occurring approximately 28 days post DMAb inoculation as quantified by ELISA. All three 10e DMAbs expressed in sera for approximately 2.5 months post DMAb administration, and their total serum concentrations varied based on construct identity (FIG. 2E). A general trend emerged that serum concentration of 10e DMAbs increased as number of germline reversion amino acid substitutions increased. As with the results seen in vitro, 10e DMAb-1 and 2 demonstrated binding to rCSP when isolated from sera, whereas 10e DMAb-3 did not elicit binding to rCSP even at the highest concentration measured (FIG. 2F).

In Vitro and In Vivo Produced 43e DMAbs Express and Bind rCSP

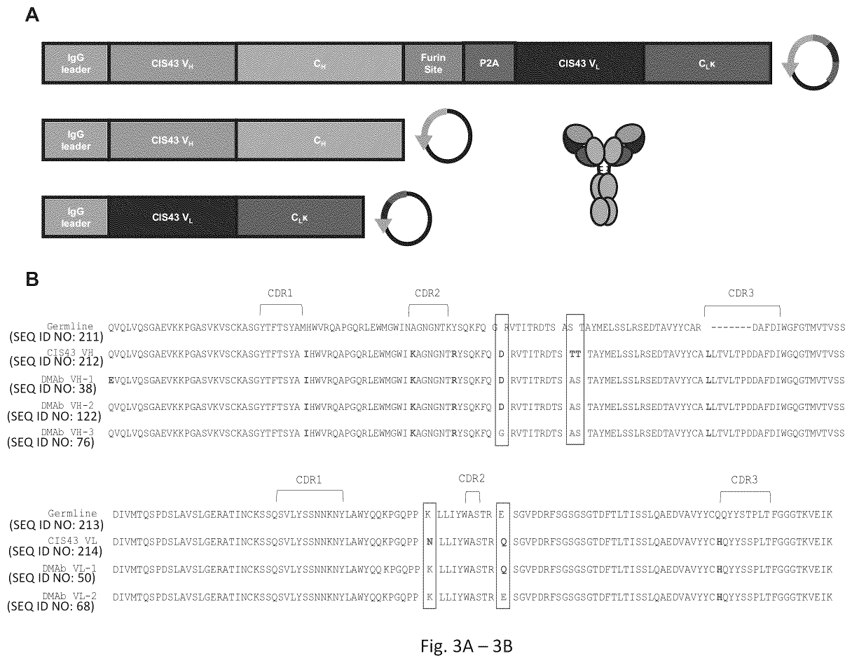

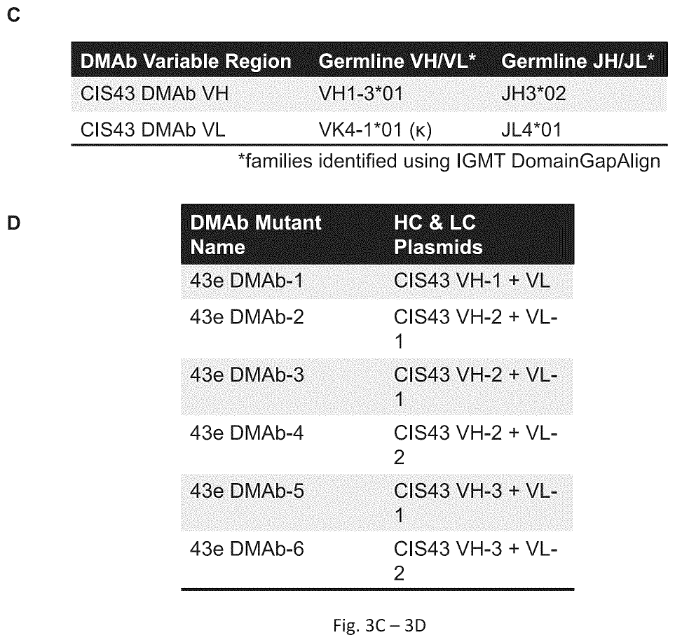

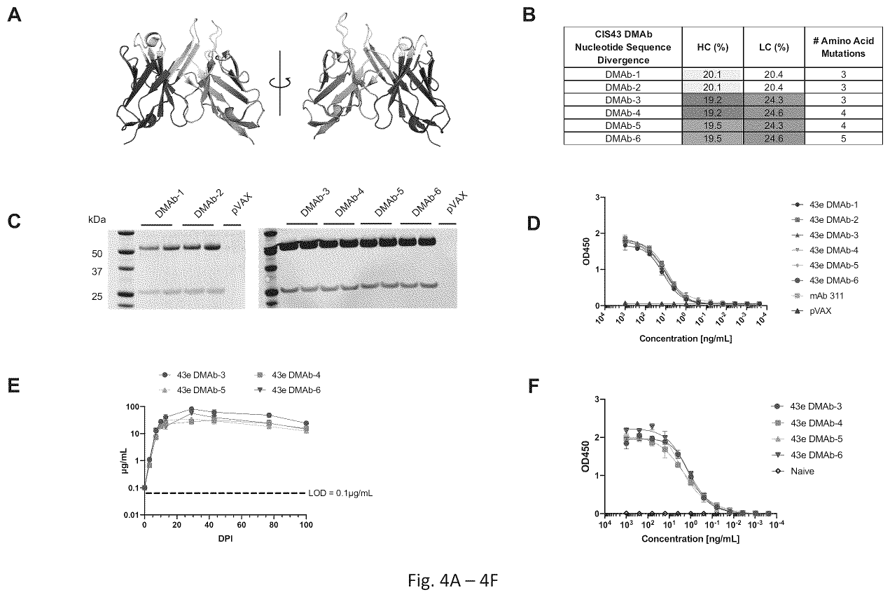

Based on the expression and binding profile observed with the 10e DMAb constructs, we wanted to test another anti-CSP mAb. As such, we designed DMAb constructs based on CIS43, an anti-CSP human mAb targeting the junctional epitope between the N terminus and NANP major repeats (Kisalu et al., 2018). In total, six CIS43-based engineered DMAb (43e DMAb) constructs were designed; two constructs (43e DMAb-1 and 2) were cloned into a single pVAX vector plasmid and four constructs (43e DMAb-3, 4, 5, and 6) were cloned into dual pVAX vector plasmids (FIG. 3A). For 43e DMAb-1 and 2, the single pVAX vector plasmid consisted of an IgG leader sequence with a furin cleavage site and porcine techovirus-1 2A peptide separating heavy and light chains. A human IgG1 constant region was cloned in after the variable regions. For 43e DMAb-3 through 6, 10e DMAbs were used as a model, and the heavy and light chain variable regions were encoded into separate plasmids flanked by IgG leader sequence and requisite constant regions. All 43e DMAbs possessed amino acid point mutations where residues in the framework region were selectively reverted to their germline configuration (FIG. 3B); this analysis was carried out using the IMGT DomainGapAlign tool. Sites throughout the variable framework region were selectively mutated. A molecular model for CIS43 was available through Protein Data Bank and was used to illustrate all sites for potential modification, but each DMAb construct may have a subset of these changes (FIG. 4A) (Kisalu et al., 2018) (PDB 6B5M). Amino acid changes were made according to specific variable heavy and light germline genes (FIG. 3C). In addition to amino acid changes, codon optimization for expression in murine and human systems was performed, changing the identity of various nucleotides in 43e DMAbs compared to heavy and light chain variable regions of WT CIS43 (FIG. 4B). Heavy chain variable region nucleotide divergence ranged from 19.2% to 20.1%, and light chain variable region nucleotide divergence varied from 20.4% to 24.6%. However, on the amino acid level, the DMAbs range from 1.3% to 2.6% different, with an average variable region amino acid divergence of 1.7%.

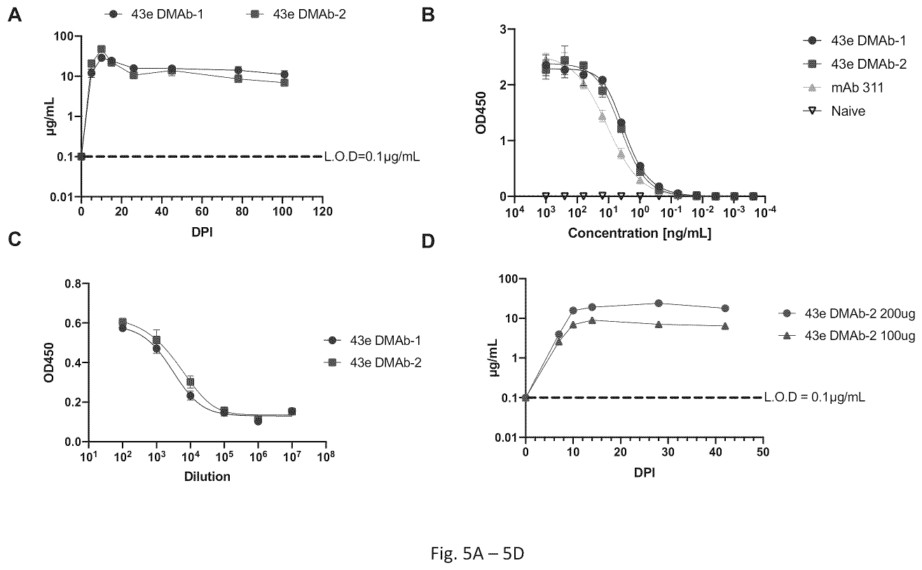

To study the expression of 43e DMAb constructs in vitro, Expi293F cells were transfected and supernatants were harvested for Western Blot analysis. Under reduced conditions, supernatants from cells transfected with all six 43e DMAbs expressed, with bands at approximately 50 kDa and 25 kDa respectively (FIG. 4C). Supernatants containing all six CIS43 demonstrated potent binding to rCSP through specific binding ELISA (FIG. 4D). In addition, supernatants containing all six 43e DMAbs exhibited binding to rCSP similar to that of mAb 311, a recombinant NANP-binding positive control. Following the in vitro screen, 43e DMAbs were administered to mice to evaluate in vivo expression and binding of in vivo produced DMAbs Sera isolated from inoculated mice (n=5 mice/group) was used for quantification of in vivo production and assessment of binding activity. All six DMAbs expressed in vivo, with variable serum concentrations and antibody half-life kinetics. Single plasmid 43e DMAb-1 and DMAb-2 exhibited peak concentration approximately 13 days post inoculation whereas dual plasmid 43e DMAb-3, 4, 5, and 6 peaked at 29 days post inoculation (FIG. 4E, FIG. 5A). Serum concentration of all six 43e DMAbs was quantified by ELISA beginning 26 days post inoculation, and we observed expression in vivo for over 3 months post a single administration. However, 43e DMAb-3 through DMAb-6 exhibited higher average serum concentration overall compared to 43e DMAb-1 and 2, with average peak concentrations of approximately 50 μg/mL compared to 38 μg/mL. In addition, we sought to understand whether sera isolated from inoculated mice retains functional activity. As assessed by binding ELISA, all six 43e DMAbs exhibit potent binding to recombinant CSP (FIG. 4F, FIG. 5B). A subset of 43e DMAbs were assessed for binding against P. falciparum sporozoites, which present CSP in its native conformation. In vivo produced 43e DMAbs bind to sporozoites as measured by ELISA (FIG. 5C). In addition, we sought to determine how modulating the dose of DNA would impact expression. 43e DMAb-2 shows a dosing effect; both constructs reach their peak expression and plateau 10 days post single DMAb inoculation, where the 200 μg group maintains approximately 2-3 fold higher expression than the 100 μg group (FIG. 5D).

10e and 43e DMAb Down Selection and In Vivo Expression without T Cell Depletion

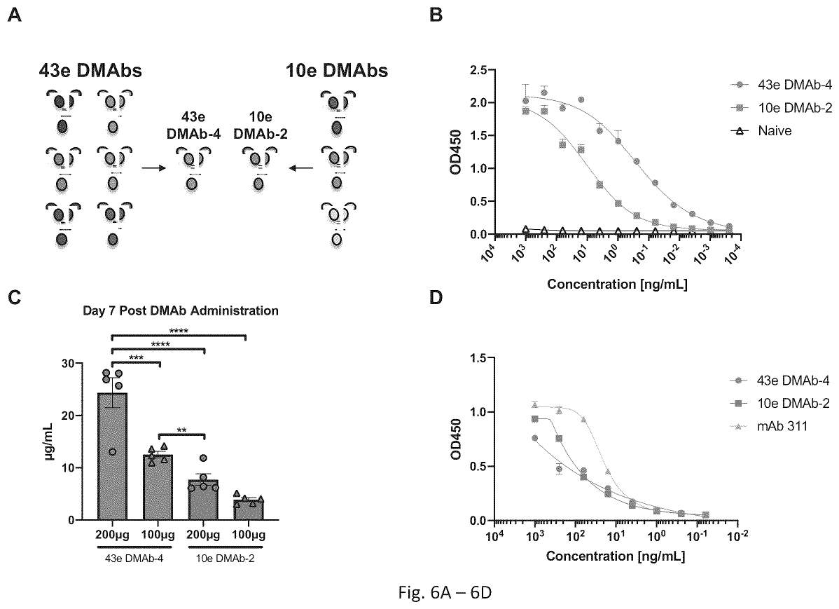

Following in vitro and in vivo screening of both 10e DMAbs, constructs were down selected. With regard to the 10e DMAb clones, all three 10e DMAbs demonstrated potent expression in vivo, with increasing serum concentration of antibody as the number of germline reversions increased. However, only 10e DMAb-1 and 2 showed binding to recombinant CSP. As such, between 10e DMAb-1 and 2, 10e DMAb-2 was chosen for down selection due to its similar binding but higher serum expression than 10e DMAb-1. Among the CIS43 clones, each 43e DMAb showed potent expression in vivo as well as binding to recombinant CSP; 43e DMAb-4 was chosen for down selection (FIG. 6A).

First, binding of in vitro produced 10e DMAb-2 and 43e DMAb-4 to rCSP were compared against each other. As assessed by ELISA, 43e DMAb-4 bound rCSP more potently than 10e DMAb-2 (FIG. 6B). In addition, how altering the dose of DNA would impact expression was examined. To do so, mice received 200 μg (100 μg each of HC and LC DNA plasmids) or 100 μg (50 μg each of HC and LC DNA plasmids) of 43e DMAb-4 and 10e DMAb-2. Quantification 43e DMAb-4 and 10e DMAb-2 in sera at Day 7 post DMAb administration was determined by ELISA and shown for both 200 μg and 100 μg doses (FIG. 6C). As determined by ANOVA, the 200 μg dose group 43e DMAb-4 exhibited significantly higher expression compared to the both the 200 μg and 100 μg dose groups for 10e DMAb-2 as well as the 100 μg dose group of 43e DMAb-4. Additionally, sera isolated from Day 7 post DMAb administration was evaluated for binding to sporozoites. 43e DMAb-4 and 10e DMAb-2 both demonstrated binding to sporozoites (FIG. 6D). However, given that 43e DMAb-4 expressed approximately three times more than 10e DMAb-2 without T cell depletion, 43e DMAb-4 was advanced to further studies in a murine mosquito bite challenge model.

43e DMAb-4 Confers Sterile Protection from Mosquito Bite Challenge Model

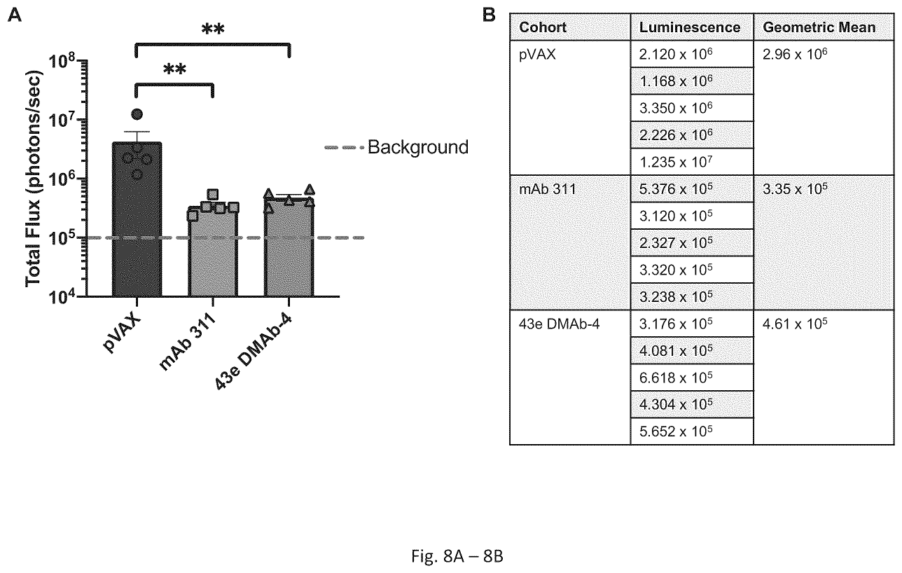

The 200 μg dose group of 43e DMAb-4 was advanced into a mosquito bite challenge model with two important functional readouts: inhibition of liver infection and blood stage parasitemia (Raghunandan et al., 2020). Seven days prior to mosquito bite challenge, mice were administered 200 μg of 43e DMAb-4 intramuscularly without CD4+ or CD8+ T cell depletion. Sixteen hours prior to challenge, 100 μg of mAb 311 was administered to the positive control group intraperitoneally. On the day of challenge, mice were exposed to the bites of 5 A. stephensi mosquitoes infected with PbPfLuc transgenic parasites. Forty-two hours post challenge, liver burden was assessed through intraperitoneal administration of D-luciferin. The bioluminescence generated by the transgenic parasites was measured for individual mice via total flux using IVIS (FIG. 7B). Both the positive control, 100 μg of recombinant mAb 311, and 200 μg of 43e DMAb-4 groups showed significantly decreased bioluminescence compared to the negative control (FIG. 8A). From the bioluminescence data, percent inhibition of liver infection can be calculated relative to infection seen in the pVAX negative control mice (FIG. 8B). To that end, mice administered 100 μg recombinant mAb 311 and 200 μg 43e DMAb-4 demonstrated 88.7% and 84.4% inhibition of liver infection respectively. Importantly, the DMAb group achieved protection as strong as the positive control, both of which were statistically significantly improved over the negative control (FIG. 7C). To evaluate blood stage parasitemia, blood smears were taken beginning four days post challenge and up to ten days post challenge (FIG. 7A). In the pVAX negative control group, all mice developed blood stage infection four days post challenge. Comparatively, 80% of mice administered 100 μg recombinant mAb 311 achieved sterile protection. Finally, 60% of mice administered 200 μg 43e DMAb-4 achieved sterile protection form blood stage parasitemia (FIG. 7D).

It is paramount to develop improved vaccines and prophylactics for malaria. The surface protein CSP is a viable sporozoite stage target (Yoshida et al., 1980). The licensed malaria vaccine RTS,S/AS01 targets CSP. However, over a median four year follow-up period, the estimate of protective efficacy for RTS,S is 28% (Olotu et al., 2016). Trials utilizing CSP-based vaccines have established a possible relationship between their efficacy and anti-CSP antibodies. A distinct correlate of protection is still unknown; these studies focus attention on protection generated by serological immunity to CSP (Foquet et al., 2014, Dobaño et al., 2019, McCall et al., 2018). In addition to vaccine candidates targeting CSP, monoclonal antibodies have been isolated from malaria survivors and vaccinees against various CSP epitopes. The central NANP repeat region of CSP is conserved and immunodominant, representing a common target for anti-CSP antibodies (Oyen et al., 2017, Tan et al., 2018a, Julien and Wardemann, 2019). However, other regions of CSP, such as the junctional epitope bridging the N terminus and NANP repeats, have elicited potent IgG antibodies. Antibodies isolated from vaccinees targeting this junctional epitope demonstrate protection in multiple murine models of malaria (Kisalu et al., 2018, Tan et al., 2018b).

Monoclonal antibodies have been evaluated for infectious disease prophylaxis or therapy. Palivizumab is an FDA-approved humanized murine mAb targeting the F protein of respiratory syncytial virus (The IMpact-RSV Study Group, 1998, Boeckh et al., 2001). In addition, TY014 is a human therapeutic mAb targeting yellow fever currently being evaluated in Phase I clinical trials (Low et al., 2020) (ClinicalTrials.gov NCT03776786). A modified version of CSP junctional-epitope targeting mAb CIS43, CIS43LS, has become the first anti-malarial mAb to enter Phase I clinical trials. CIS43LS contains mutations designed to increase plasma half-life, and some participants will undergo CHMI to assess protective efficacy (ClinicalTrials.gov NCT04206332).

It has been demonstrated that human mAbs administered using a synthetic DNA platform can elicit expression both in vitro and in vivo. Further, amino acid modifications, as well as encoding heavy and light chains on dual plasmids, allowed for enhanced DMAb expression, showing the importance of in silico modifications to enhance systemic human IgG expression in this in vivo system. In addition, DMAbs show binding to rCSP as well as sporozoites, which present CSP in its native conformation. Following down selection, 43e DMAb-4 was chosen for evaluation in a rigorous mosquito bite challenge model (Oyen et al., 2017, Pholcharee et al., 2020, Raghunandan et al., 2020). As a control, 200 μg 43e DMAb-4 was compared to 100 μg recombinant human monoclonal antibody mAb 311; it has been shown that 311 confers dose-dependent protection in rigorous mosquito bite challenge models (Raghunandan et al., 2020). 43e DMAb-4, administered 7 days prior to challenge, elicited sterile protection similar to recombinant mAb 311 administered 16 hours before challenge. 43e DMAb-4 required lower serum titer levels than WT CIS43 for comparable protective efficacy (Kisalu et al., 2018, Wang et al., 2020). These differences may result from the nature of administration, assembly, and secretion. Recombinant mAbs, which are produced in vitro using human cells, are being constantly degraded in vivo post infusion. However, DNA-encoded mAbs are being assembled and secreted from transfected cells in vivo, enabling a unique production and degradation rate.

Previous studies have demonstrated that DMAb cocktails can be delivered to target multiple different antigens on the same pathogen (Flingai et al., 2015, Patel et al., 2018). Such an approach could be employed to target multiple antigens in the same or different stages in the parasitic life cycle. While monoclonal antibody infusions may provide short term protection for an activity such as travel, an anti-PfCSP DMAb such as 43e DMAb-4 could be useful due to its potential for long-term transient expression. In addition, a combination vaccine and immunotherapy approach using a vaccine with a DMAb could provide short and long term field immunity, as initial studies have demonstrated with a DNA vaccine and DMAb targeting chikungunya virus (Muthumani et al., 2016).

This Example demonstrates the potential of a synthetic DNA delivered mAb biologic targeting PfCSP for malaria prophylaxis. This is the first demonstration of a DNA-encoded antibody in the malaria space, providing benefits such as prolonged serum half-life as well as protection in a transient fashion. Leveraging the safety profile of DNA vaccines in humans as well as recent advances in DNA vaccine technology, the DMAb platform allows for rapid design, in silico modifications, and in vivo production of antibody. Prolonged systemic expression of human IgG could be of value to avoid various production costs and challenges with recombinant mAb development and administration, and provide a valuable addition for malaria elimination.

### Example 2

To further evaluate the protective capacity of different DMAb constructs, liver burden and parasitemia were measured simultaneously in immunized mice. Groups of 7 mice were immunized on day 0 with different constructs of DNA displaying different epitopes of Plasmodium falciparum: circumsporozoite protein (CSP), negative control pVax plasmid, or positive control monoclonal antibody AB-311.

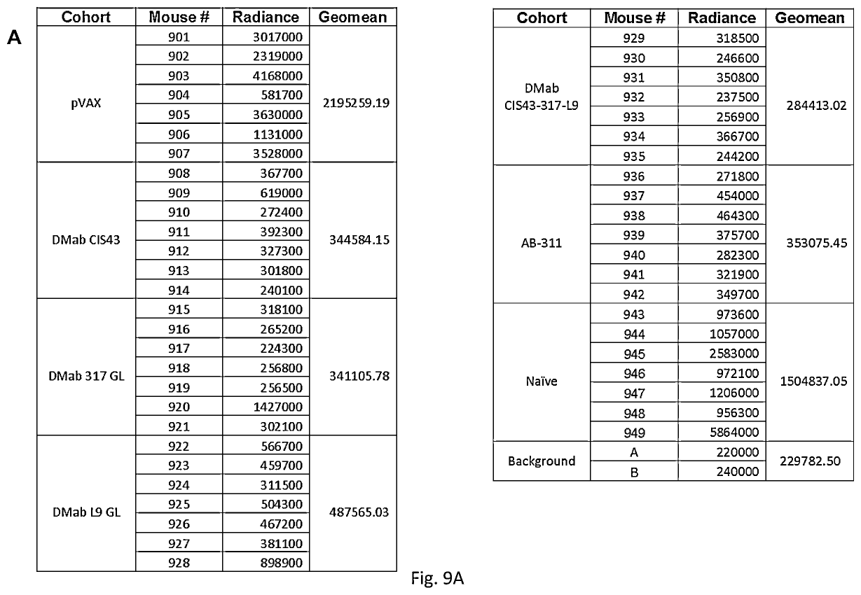

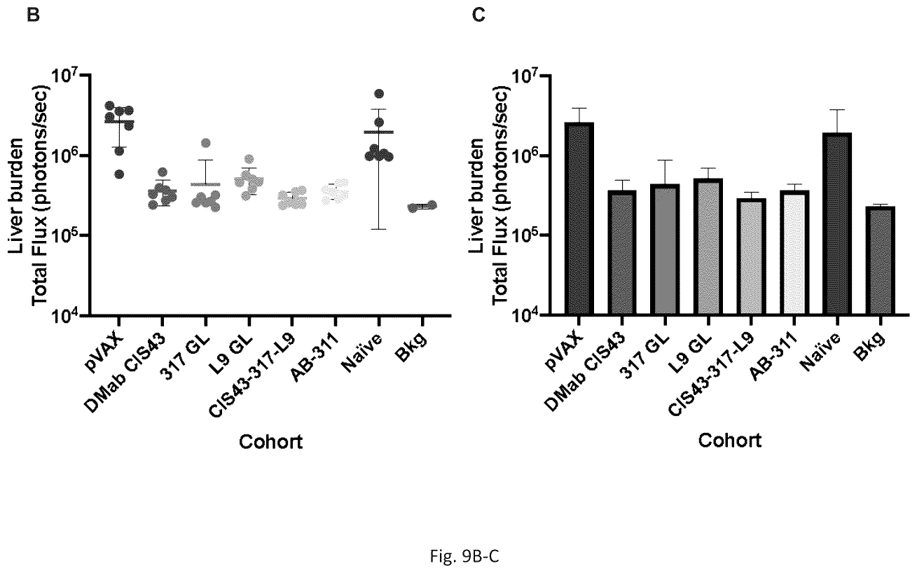

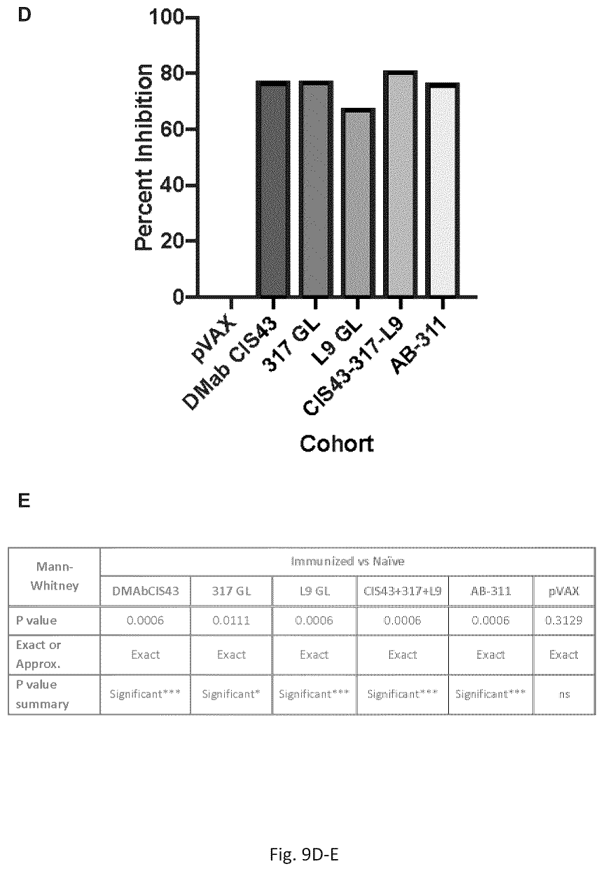

To evaluate liver burden, three weeks after the immunization mice were challenged with a transgenic, chimeric Plasmodium berghei parasite expressing the full-length P. falciparum CSP and luciferase (PbPf-GFPLuc). Forty-two hours after challenge, mice were injected with 100 μl of D-Luciferin (30 mg/mL), anesthetized with isoflurane and imaged with the IVIS spectrum to measure the bioluminescence of the D-Luciferin reactant processed by Luciferase expressed by the chimeric parasites. The results in FIG. 9 demonstrate a significant reduction in liver burden of mice immunized with the DMAb constructs displaying different P. falciparum CSP epitopes, which were comparable in protection to positive control monoclonal antibody AB-311.

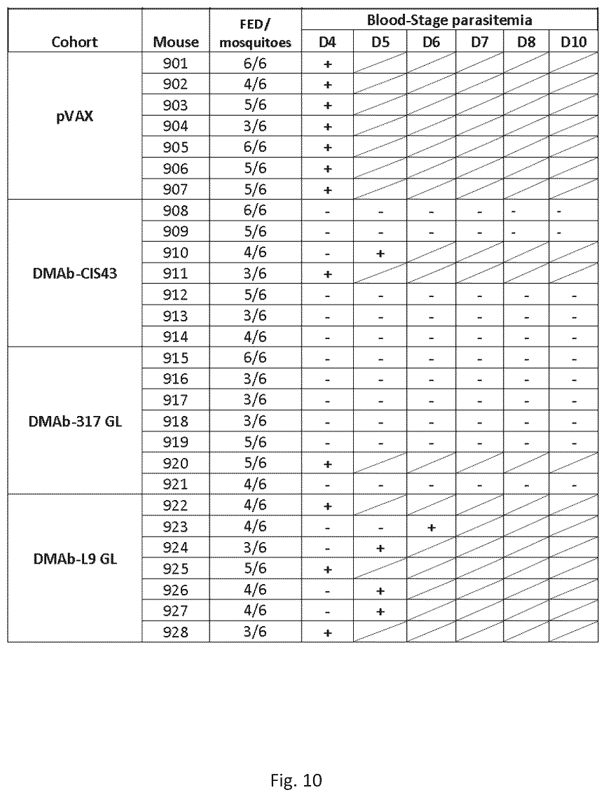

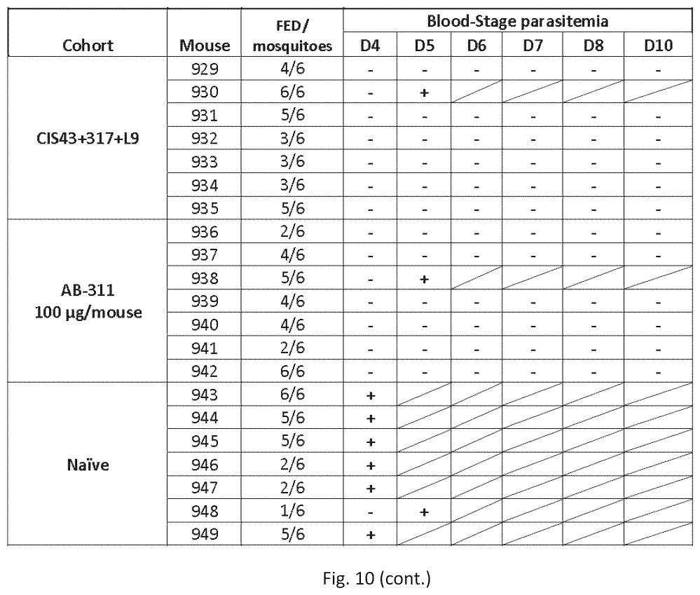

To evaluate parasitemia, first a preliminary experiment was performed to determine the approximate probability of mosquito infection after feeding on mice infected with chimeric Plasmodium berghei parasite expressing the full-length P. falciparum CSP (Pb-Pf). Twenty days after blood feeding on Pb-Pf infected mice, 80% of the mosquitoes were infected (16 out of 20 infected salivary glands). Based on this calculation, it was determined that 6 mosquitoes were needed to challenge mice with ˜5 infected mosquito bites. Next, mice were immunized with above-mentioned DNA constructs and controls, anesthetized and challenged with Pb-Pf exposed mosquitoes for approximately 10 minutes. On days four through ten post-challenge, mice were evaluated for blood-stage parasitemia. The results in FIG. 10 demonstrate significant protection against blood-stage parasitemia in mice immunized with the DMAb constructs displaying different P. falciparum CSP epitopes. Notably, immunizing with DMAb 317 GL or all three DMAb constructs provided comparable protection to immunization with positive control monoclonal antibody AB-311.

### Example 3

A sequence listing provided herewith contains a list of sequences as described in Tables 1-30 below. NA: nucleic acid sequence; AA: amino acid sequence; VH: variable heavy chain region; VL: variable light chain region; P2A: porcine teschovirus-1 2A peptide.

TABLE 1
10e DMAb-1

| NA/AA | Type | SEQ ID NO |
|---|---|---|
| NA | Heavy + Light | 1 |
| AA | Heavy + Light | 2 |
| NA | Heavy Chain | 3 |
| AA | Heavy Chain | 4 |
| NA | Light Chain | 5 |
| AA | Light Chain | 6 |
| NA | MGU10 VH | 7 |
| AA | MGU10 VH | 8 |
| NA | MGU10 VL | 9 |
| AA | MGU10 VL | 10 |
| NA | Heavy Constant | 11 |
| AA | Heavy Constant | 12 |
| NA | Light Constant | 13 |
| AA | Light Constant | 14 |

TABLE 2
10e DMAb-2

| NA/AA | Type | SEQ ID NO |
|---|---|---|
| NA | Heavy + Light | 15 |
| AA | Heavy + Light | 16 |
| NA | Heavy Chain | 17 |
| AA | Heavy Chain | 18 |
| NA | Light Chain | 19 |
| AA | Light Chain | 20 |
| NA | MGU10 VH-1 | 21 |
| AA | MGU10 VH-1 | 22 |
| NA | MGU10 VL | 9 |
| AA | MGU10 VL | 10 |
| NA | Heavy Constant | 11 |
| AA | Heavy Constant | 12 |
| NA | Light Constant | 13 |
| AA | Light Constant | 14 |

TABLE 3
10e DMAb-3

| NA/AA | Type | SEQ ID NO |
|---|---|---|
| NA | Heavy + Light | 23 |
| AA | Heavy + Light | 24 |
| NA | Heavy Chain | 25 |
| AA | Heavy Chain | 26 |
| NA | Light Chain | 27 |
| AA | Light Chain | 28 |
| NA | MGU10 VH-1 | 21 |
| AA | MGU10 VH-1 | 22 |
| NA | MGU10 VL-1 | 29 |
| AA | MGU10 VL-1 | 30 |
| NA | Heavy Constant | 11 |
| AA | Heavy Constant | 12 |
| NA | Light Constant | 13 |
| AA | Light Constant | 14 |

TABLE 4
43e DMAb-1

| NA/AA | Type | SEQ ID NO |
|---|---|---|
| NA | Heavy + Light | 31 |
| AA | Heavy + Light | 32 |
| NA | Heavy Chain | 33 |
| AA | Heavy Chain | 34 |
| NA | Light Chain | 35 |
| AA | Light Chain | 36 |
| NA | CIS43 VH-1 | 37 |
| AA | CIS43 VH-1 | 38 |
| NA | CIS43 VL | 39 |
| AA | CIS43 VL | 40 |
| NA | Heavy Constant | 11 |
| AA | Heavy Constant | 12 |
| NA | Light Constant | 41 |
| AA | Light Constant | 42 |

TABLE 5
43e DMAb-2

| NA/AA | Type | SEQ ID NO |
|---|---|---|
| NA | Heavy + Light | 43 |
| AA | Heavy + Light | 44 |
| NA | Heavy Chain | 45 |
| AA | Heavy Chain | 46 |
| NA | Light Chain | 47 |
| AA | Light Chain | 48 |
| NA | CIS43 VH-2 | 37 |
| AA | CIS43 VH-2 | 38 |
| NA | CIS43 VL-1 | 49 |
| AA | CIS43 VL-1 | 50 |
| NA | Heavy Constant | 11 |
| AA | Heavy Constant | 12 |
| NA | Light Constant | 41 |
| AA | Light Constant | 42 |

TABLE 6
43e DMAb-3

| NA/AA | Type | SEQ ID NO |
|---|---|---|
| NA | Heavy + Light | 51 |
| AA | Heavy + Light | 52 |
| NA | Heavy Chain | 53 |
| AA | Heavy Chain | 54 |
| NA | Light Chain | 55 |
| AA | Light Chain | 56 |
| NA | CIS43 VH-2 | 57 |
| AA | CIS43 VH-2 | 58 |
| NA | CIS43 VL-1 | 49 |
| AA | CIS43 VL-1 | 50 |
| NA | Heavy Constant | 11 |
| AA | Heavy Constant | 12 |
| NA | Light Constant | 59 |
| AA | Light Constant | 60 |

TABLE 7
43e DMAb-4

| NA/AA | Type | SEQ ID NO |
|---|---|---|
| NA | Heavy + Light | 61 |
| AA | Heavy + Light | 62 |
| NA | Heavy Chain | 63 |
| AA | Heavy Chain | 64 |
| NA | Light Chain | 65 |
| AA | Light Chain | 66 |
| NA | CIS43 VH-2 | 57 |
| AA | CIS43 VH-2 | 58 |
| NA | CIS43 VL-2 | 67 |
| AA | CIS43 VL-2 | 68 |
| NA | Heavy Constant | 11 |
| AA | Heavy Constant | 12 |
| NA | Light Constant | 59 |
| AA | Light Constant | 60 |

TABLE 8
43e DMAb-5

| NA/AA | Type | SEQ ID NO |
|---|---|---|
| NA | Heavy + Light | 69 |
| AA | Heavy + Light | 70 |
| NA | Heavy Chain | 71 |
| AA | Heavy Chain | 72 |
| NA | Light Chain | 73 |
| AA | Light Chain | 74 |
| NA | CIS43 VH-3 | 75 |
| AA | CIS43 VH-3 | 76 |
| NA | CIS43 VL-1 | 49 |
| AA | CIS43 VL-1 | 50 |
| NA | Heavy Constant | 11 |
| AA | Heavy Constant | 12 |
| NA | Light Constant | 59 |
| AA | Light Constant | 60 |

TABLE 9
43e DMAb-6

| NA/AA | Type | SEQ ID NO |
|---|---|---|
| NA | Full | 77 |
| AA | Full | 78 |
| NA | Heavy Chain | 79 |
| AA | Heavy Chain | 80 |
| NA | Light Chain | 81 |
| AA | Light Chain | 82 |
| NA | CIS43 VH-3 | 75 |
| AA | CIS43 VH-3 | 76 |
| NA | CIS43 VL-2 | 67 |
| AA | CIS43 VL-2 | 68 |
| NA | Heavy Constant | 11 |
| AA | Heavy Constant | 12 |
| NA | Light Constant | 59 |
| AA | Light Constant | 60 |

TABLE 10
317 DMAb-1

| NA/AA | Type | SEQ ID NO |
|---|---|---|
| NA | Full | 93 |
| AA | Full | 94 |
| NA | Heavy Chain | 95 |
| AA | Heavy Chain | 96 |
| NA | Light Chain | 97 |
| AA | Light Chain | 98 |
| NA | 317 VH-1 | 99 |
| AA | 317 VH-1 | 100 |
| NA | 317 VL-1 | 101 |
| AA | 317 VL-1 | 102 |
| NA | Heavy Constant | 11 |
| AA | Heavy Constant | 12 |
| NA | Light Constant | 41 |
| AA | Light Constant | 42 |

TABLE 11
317 DMAb-2

| NA/AA | Type | SEQ ID NO |
|---|---|---|
| NA | Full | 103 |
| AA | Full | 104 |
| NA | Heavy Chain | 95 |
| AA | Heavy Chain | 96 |
| NA | Light Chain | 105 |
| AA | Light Chain | 106 |
| NA | 317 VH-1 | 99 |
| AA | 317 VH-1 | 100 |
| NA | 317 VL-2 | 107 |
| AA | 317 VL-2 | 108 |
| NA | Heavy Constant | 11 |
| AA | Heavy Constant | 12 |
| NA | Light Constant | 41 |
| AA | Light Constant | 42 |

TABLE 12
317 DMAb-3

| NA/AA | Type | SEQ ID NO |
|---|---|---|
| NA | Full | 109 |
| AA | Full | 110 |
| NA | Heavy Chain | 111 |
| AA | Heavy Chain | 112 |
| NA | Light Chain | 97 |
| AA | Light Chain | 98 |
| NA | 317 VH-2 | 113 |
| AA | 317 VH-2 | 114 |
| NA | 317 VL-1 | 101 |
| AA | 317 VL-1 | 102 |
| NA | Heavy Constant | 11 |
| AA | Heavy Constant | 12 |
| NA | Light Constant | 41 |
| AA | Light Constant | 42 |

TABLE 13
317 DMAb-4

| NA/AA | Type | SEQ ID NO |
|---|---|---|
| NA | Full | 115 |
| AA | Full | 116 |
| NA | Heavy Chain | 111 |
| AA | Heavy Chain | 112 |
| NA | Light Chain | 105 |
| AA | Light Chain | 106 |
| NA | 317 VH-2 | 113 |
| AA | 317 VH-2 | 114 |
| NA | 317 VL-2 | 107 |
| AA | 317 VL-2 | 108 |
| NA | Heavy Constant | 11 |
| AA | Heavy Constant | 12 |
| NA | Light Constant | 41 |
| AA | Light Constant | 42 |

TABLE 14
43e E430G DMAb-1

| NA/AA | Type | SEQ ID NO |
|---|---|---|
| NA | Full | 117 |
| AA | Full | 118 |
| NA | Heavy Chain | 119 |
| AA | Heavy Chain | 120 |
| NA | Light Chain | 97 |
| AA | Light Chain | 98 |
| NA | 43e CV VH-1 | 121 |
| AA | 43e CV VH-1 | 122 |
| NA | 43e CV VL-1 | 101 |
| AA | 43e CV VL-1 | 102 |
| NA | Heavy Constant | 123 |
| AA | Heavy Constant | 124 |
| NA | Light Constant | 41 |
| AA | Light Constant | 42 |

TABLE 15
43e E430G DMAb-2

| NA/AA | Type | SEQ ID NO |
|---|---|---|
| NA | Full | 125 |
| AA | Full | 126 |
| NA | Heavy Chain | 119 |
| AA | Heavy Chain | 120 |
| NA | Light Chain | 105 |
| AA | Light Chain | 106 |
| NA | 43e CV VH-1 | 121 |
| AA | 43e CV VH-1 | 122 |
| NA | 43e CV VL-2 | 107 |
| AA | 43e CV VL-2 | 108 |
| NA | Heavy Constant | 123 |
| AA | Heavy Constant | 124 |
| NA | Light Constant | 41 |
| AA | Light Constant | 42 |

TABLE 16
43e D270A/K322A DMAb-1

| NA/AA | Type | SEQ ID NO |
|---|---|---|
| NA | Full | 127 |
| AA | Full | 128 |
| NA | Heavy Chain | 129 |
| AA | Heavy Chain | 130 |
| NA | Light Chain | 97 |
| AA | Light Chain | 98 |
| NA | 43e CV VH-2 | 131 |
| AA | 43e CV VH-2 | 132 |
| NA | 43e CV VL-1 | 101 |
| AA | 43e CV VL-1 | 102 |
| NA | Heavy Constant | 133 |
| AA | Heavy Constant | 134 |
| NA | Light Constant | 41 |
| AA | Light Constant | 42 |

TABLE 17
43e D270A/K322A DMAb-2

| NA/AA | Type | SEQ ID NO |
|---|---|---|
| NA | Full | 135 |
| AA | Full | 136 |
| NA | Heavy Chain | 129 |
| AA | Heavy Chain | 130 |
| NA | Light Chain | 105 |
| AA | Light Chain | 106 |
| NA | 43e CV VH-2 | 131 |
| AA | 43e CV VH-2 | 132 |
| NA | 43e CV VL-2 | 107 |
| AA | 43e CV VL-2 | 108 |
| NA | Heavy Constant | 133 |
| AA | Heavy Constant | 134 |
| NA | Light Constant | 41 |
| AA | Light Constant | 42 |

TABLE 18
L9 DMAb-1

| NA/AA | Type | SEQ ID NO |
|---|---|---|
| NA | Full | 137 |
| AA | Full | 138 |
| NA | Heavy Chain | 139 |
| AA | Heavy Chain | 140 |
| NA | Light Chain | 141 |
| AA | Light Chain | 142 |
| NA | L9 VH-1 | 143 |
| AA | L9 VH-1 | 144 |
| NA | L9 VL-1 | 145 |
| AA | L9 VL-1 | 146 |
| NA | Heavy Constant | 147 |
| AA | Heavy Constant | 148 |
| NA | Light Constant | 149 |
| AA | Light Constant | 150 |

TABLE 19
L9 DMAb-2

| NA/AA | Type | SEQ ID NO |
|---|---|---|
| NA | Full | 151 |
| AA | Full | 152 |
| NA | Heavy Chain | 139 |
| AA | Heavy Chain | 140 |
| NA | Light Chain | 153 |
| AA | Light Chain | 154 |
| NA | L9 VH-1 | 143 |
| AA | L9 VH-1 | 144 |
| NA | L9 VL-2 | 155 |
| AA | L9 VL-2 | 156 |
| NA | Heavy Constant | 147 |
| AA | Heavy Constant | 148 |
| NA | Light Constant | 157 |
| AA | Light Constant | 158 |

TABLE 20
L9 DMAb-3

| NA/AA | Type | SEQ ID NO |
|---|---|---|
| NA | Full | 159 |
| AA | Full | 160 |
| NA | Heavy Chain | 161 |
| AA | Heavy Chain | 162 |
| NA | Light Chain | 141 |
| AA | Light Chain | 142 |
| NA | L9 VH-2 | 163 |
| AA | L9 VH-2 | 164 |
| NA | L9 VL-1 | 145 |
| AA | L9 VL-1 | 146 |
| NA | Heavy Constant | 165 |
| AA | Heavy Constant | 166 |
| NA | Light Constant | 149 |
| AA | Light Constant | 150 |

TABLE 21
L9 DMAb-4

| NA/AA | Type | SEQ ID NO |
|---|---|---|
| NA | Full | 167 |
| AA | Full | 168 |
| NA | Heavy Chain | 161 |
| AA | Heavy Chain | 162 |
| NA | Light Chain | 153 |
| AA | Light Chain | 154 |
| NA | L9 VH-2 | 163 |
| AA | L9 VH-2 | 164 |
| NA | L9 VL-2 | 155 |
| AA | L9 VL-2 | 156 |
| NA | Heavy Constant | 165 |
| AA | Heavy Constant | 166 |
| NA | Light Constant | 157 |
| AA | Light Constant | 158 |

TABLE 22
L9 DMAb-1 E430G

| NA/AA | Type | SEQ ID NO |
|---|---|---|
| NA | Full | 169 |
| AA | Full | 170 |
| NA | Heavy Chain | 171 |
| AA | Heavy Chain | 172 |
| NA | Light Chain | 141 |
| AA | Light Chain | 142 |
| NA | L9 VH-3 | 173 |
| AA | L9 VH-3 | 174 |
| NA | L9 VL-1 | 145 |
| AA | L9 VL-1 | 146 |
| NA | Heavy Constant | 175 |
| AA | Heavy Constant | 176 |
| NA | Light Constant | 149 |
| AA | Light Constant | 150 |

TABLE 23
L9 DMAb-2 E430G

| NA/AA | Type | SEQ ID NO |
|---|---|---|
| NA | Full | 177 |
| AA | Full | 178 |
| NA | Heavy Chain | 171 |
| AA | Heavy Chain | 172 |
| NA | Light Chain | 153 |
| AA | Light Chain | 154 |
| NA | L9 VH-3 | 173 |
| AA | L9 VH-3 | 174 |
| NA | L9 VL-2 | 155 |
| AA | L9 VL-2 | 156 |
| NA | Heavy Constant | 175 |
| AA | Heavy Constant | 176 |
| NA | Light Constant | 157 |
| AA | Light Constant | 158 |

TABLE 24
L9 DMAb-3 E430G

| NA/AA | Type | SEQ ID NO |
|---|---|---|
| NA | Full | 179 |
| AA | Full | 180 |
| NA | Heavy Chain | 181 |
| AA | Heavy Chain | 182 |
| NA | Light Chain | 141 |
| AA | Light Chain | 142 |
| NA | L9 VH-4 | 183 |
| AA | L9 VH-4 | 184 |
| NA | L9 VL-1 | 145 |
| AA | L9 VL-1 | 146 |
| NA | Heavy Constant | 185 |
| AA | Heavy Constant | 186 |
| NA | Light Constant | 149 |
| AA | Light Constant | 150 |

TABLE 25
L9 DMAb-4 E430G

| NA/AA | Type | SEQ ID NO |
|---|---|---|
| NA | Full | 187 |
| AA | Full | 188 |
| NA | Heavy Chain | 181 |
| AA | Heavy Chain | 182 |
| NA | Light Chain | 153 |
| AA | Light Chain | 154 |
| NA | L9 VH-4 | 183 |
| AA | L9 VH-4 | 184 |
| NA | L9 VL-2 | 155 |
| AA | L9 VL-2 | 156 |
| NA | Heavy Constant | 185 |
| AA | Heavy Constant | 186 |
| NA | Light Constant | 157 |
| AA | Light Constant | 158 |

TABLE 26
L9 DMAb-1 D270A/K322A

| NA/AA | Type | SEQ ID NO |
|---|---|---|
| NA | Full | 189 |
| AA | Full | 190 |
| NA | Heavy Chain | 191 |
| AA | Heavy Chain | 192 |
| NA | Light Chain | 141 |
| AA | Light Chain | 142 |
| NA | L9 VH-5 | 193 |
| AA | L9 VH-5 | 194 |
| NA | L9 VL-1 | 145 |
| AA | L9 VL-1 | 146 |
| NA | Heavy Constant | 195 |
| AA | Heavy Constant | 196 |
| NA | Light Constant | 149 |
| AA | Light Constant | 150 |

TABLE 27
L9 DMAb-2 D270A/K322A

| NA/AA | Type | SEQ ID NO |
|---|---|---|
| NA | Full | 197 |
| AA | Full | 198 |
| NA | Heavy Chain | 191 |
| AA | Heavy Chain | 192 |
| NA | Light Chain | 153 |
| AA | Light Chain | 154 |
| NA | L9 VH-5 | 193 |
| AA | L9 VH-5 | 194 |
| NA | L9 VL-2 | 155 |
| AA | L9 VL-2 | 156 |
| NA | Heavy Constant | 195 |
| AA | Heavy Constant | 196 |
| NA | Light Constant | 157 |
| AA | Light Constant | 158 |

TABLE 28
L9 DMAb-3 D270A/K322A

| NA/AA | Type | SEQ ID NO |
|---|---|---|
| NA | Full | 199 |
| AA | Full | 200 |
| NA | Heavy Chain | 201 |
| AA | Heavy Chain | 202 |
| NA | Light Chain | 141 |
| AA | Light Chain | 142 |
| NA | L9 VH-6 | 203 |
| AA | L9 VH-6 | 204 |
| NA | L9 VL-1 | 145 |
| AA | L9 VL-1 | 146 |
| NA | Heavy Constant | 205 |
| AA | Heavy Constant | 206 |
| NA | Light Constant | 149 |
| AA | Light Constant | 150 |

TABLE 29
L9 DMAb-4 D270A/K322A

| NA/AA | Type | SEQ ID NO |
|---|---|---|
| NA | Full | 207 |
| AA | Full | 208 |
| NA | Heavy Chain | 201 |
| AA | Heavy Chain | 202 |
| NA | Light Chain | 153 |
| AA | Light Chain | 154 |
| NA | L9 VH-6 | 203 |
| AA | L9 VH-6 | 204 |
| NA | L9 VL-2 | 155 |
| AA | L9 VL-2 | 156 |
| NA | Heavy Constant | 205 |
| AA | Heavy Constant | 206 |
| NA | Light Constant | 157 |
| AA | Light Constant | 158 |

TABLE 30
Cleavage and signal peptides

| Name | NA/AA | SEQ ID NO |
|---|---|---|
| Furin | NA | 83 |
| Furin | AA | 84 |
| P2A | NA | 85 |
| peptide | | |
| P2A | AA | 86 |
| peptide | | |
| Signal | NA | 87 |
| Peptide | | |
| 1 | | |
| Signal | AA | 88 |
| Peptide | | |
| 1 | | |
| Signal | NA | 89 |
| Peptide | | |
| 2 | | |
| Signal | AA | 90 |
| Peptide | | |
| 2 | | |
| Signal | NA | 91 |
| Peptide | | |
| 3 | | |
| Signal | AA | 92 |
| Peptide | | |
| 3 | | |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A nucleic acid molecule comprising one or more nucleotide sequence encoding one or more polypeptide, wherein said one or more polypeptide is one or more selected from the group consisting of:
a) a heavy chain polypeptide of an anti-Plasmodium circumsporozoite protein (CSP) antibody; and
b) a light chain polypeptide of an anti-Plasmodium CSP antibody.

2. The nucleic acid molecule of claim 1, wherein said nucleotide sequence encoding the heavy chain polypeptide comprises a nucleotide sequence encoding a variable heavy chain region, wherein said variable heavy chain region comprises an amino acid sequence that is at least 95% identical to an amino acid sequence selected from the group consisting of: SEQ ID NO: 8, SEQ ID NO: 22, SEQ ID NO: 38, SEQ ID NO: 58, SEQ ID NO: 76, SEQ ID NO: 100, SEQ ID NO: 114, SEQ ID NO: 122, SEQ ID NO: 132, SEQ ID NO: 144, SEQ ID NO: 164, SEQ ID NO: 174, SEQ ID NO: 184, SEQ ID NO:
194, and SEQ ID NO: 204.

3. The nucleic acid molecule of claim 2, wherein said nucleotide sequence encoding a variable heavy chain region comprises a nucleotide sequence that is at least 95% identical to a nucleotide sequence selected from the group consisting of: SEQ ID NO: 7, SEQ ID NO: 21, SEQ ID NO: 37, SEQ ID NO: 57, SEQ ID NO: 75, SEQ ID NO: 99, SEQ ID NO: 113, SEQ ID NO: 121, SEQ ID NO: 131, SEQ ID NO: 143, SEQ ID NO:
163, SEQ ID NO: 173, SEQ ID NO: 183, SEQ ID NO: 193, and SEQ ID NO: 203.

4. The nucleic acid molecule of claim 2, wherein said heavy chain polypeptide comprises an amino acid sequence that is at least 95% identical to an amino acid sequence selected from the group consisting of: SEQ ID NO: 4, SEQ ID NO: 18, SEQ ID NO: 26, SEQ ID NO: 34, SEQ ID NO: 46, SEQ ID NO: 54, SEQ ID NO: 64, SEQ ID NO: 72, SEQ ID NO: 80, SEQ ID NO: 96, SEQ ID NO: 112, SEQ ID NO: 120, SEQ ID NO: 130, SEQ ID NO: 140, SEQ ID NO: 162, SEQ ID NO: 172, SEQ ID NO: 182, SEQ ID NO: 192, and SEQ ID NO: 202.

5. The nucleic acid molecule of claim 2, wherein said nucleotide sequence encoding the heavy chain polypeptide comprises a nucleotide sequence that is at least 95% identical to a nucleotide sequence selected from the group consisting of: SEQ ID NO: 3, SEQ ID NO: 17, SEQ ID NO: 25, SEQ ID NO: 33, SEQ ID NO: 45, SEQ ID NO: 53, SEQ ID NO: 63, SEQ ID NO: 71, SEQ ID NO: 79, SEQ ID NO: 95, SEQ ID NO: 111, SEQ ID NO: 119, SEQ ID NO: 129, SEQ ID NO: 139, SEQ ID NO: 161, SEQ ID NO: 171, SEQ ID NO: 181, SEQ ID NO: 191, and SEQ ID NO: 201.

6. The nucleic acid molecule of claim 1, wherein said nucleotide sequence encoding the light chain polypeptide comprises a nucleotide sequence encoding a variable light chain region, wherein said variable light chain region comprises an amino acid sequence that is at least 95% identical to an amino acid sequence selected from the group consisting of: SEQ ID NO: 10, SEQ ID NO: 30, SEQ ID NO: 40, SEQ ID NO: 50, SEQ ID NO: 68, SEQ ID NO: 102, SEQ ID NO: 108, SEQ ID NO: 146, and SEQ ID NO: 156.

7. The nucleic acid molecule of claim 6, wherein said nucleotide sequence encoding a variable light chain region comprises a nucleotide sequence that is at least 95% identical to a nucleotide sequence selected from the group consisting of: SEQ ID NO: 9, SEQ ID NO: 29, SEQ ID NO: 39, SEQ ID NO: 49, SEQ ID NO: 67, SEQ ID NO: 101, SEQ ID NO: 107, SEQ ID NO: 145, and SEQ ID NO: 155.

8. The nucleic acid molecule of claim 6, wherein said light chain polypeptide comprises an amino acid sequence that is at least 95% identical to an amino acid sequence selected from the group consisting of: SEQ ID NO: 6, SEQ ID NO: 20, SEQ ID NO: 28, SEQ ID NO: 36, SEQ ID NO: 48, SEQ ID NO: 56, SEQ ID NO: 66, SEQ ID NO: 74, SEQ ID NO: 82, SEQ ID NO: 98, SEQ ID NO: 106, SEQ ID NO: 142, and SEQ ID NO: 154.

9. The nucleic acid molecule of claim 6, wherein said nucleotide sequence encoding the light chain polypeptide comprises a nucleotide sequence that is at least 95% identical to a nucleotide sequence selected from the group consisting of: SEQ ID NO: 5, SEQ ID NO: 19, SEQ ID NO: 27, SEQ ID NO: 35, SEQ ID NO: 47, SEQ ID NO: 55, SEQ ID NO: 65, SEQ ID NO: 73, SEQ ID NO: 81, SEQ ID NO: 97, SEQ ID NO: 105, SEQ ID NO: 141, and SEQ ID NO: 153.

10. A composition comprising at least two nucleic acid molecules wherein:
a) at least one nucleic acid molecule comprises a nucleotide sequence encoding a heavy chain polypeptide of an anti-Plasmodium CSP antibody; and
b) at least one nucleic acid molecule comprises a nucleotide sequence encoding a light chain polypeptide of an anti-Plasmodium CSP antibody.

11. The nucleic acid molecule of claim 10, wherein said nucleotide sequence encoding the heavy chain polypeptide comprises a nucleotide sequence encoding a variable heavy chain region, wherein said variable heavy chain region comprises an amino acid sequence that is at least 95% identical to an amino acid sequence selected from the group consisting of: SEQ ID NO: 8, SEQ ID NO: 22, SEQ ID NO: 38, SEQ ID NO: 58, SEQ ID NO: 76, SEQ ID NO: 100, SEQ ID NO: 114, SEQ ID NO: 122, SEQ ID NO: 132, SEQ ID NO: 144, SEQ ID NO: 164, SEQ ID NO: 174, SEQ ID NO: 184, SEQ ID NO: 194, and SEQ ID NO: 204.

12. The nucleic acid molecule of claim 11, wherein said nucleotide sequence encoding a variable heavy chain region comprises a nucleotide sequence that is at least 95% identical to a nucleotide sequence selected from the group consisting of: SEQ ID NO: 7, SEQ ID NO: 21, SEQ ID NO: 37, SEQ ID NO: 57, SEQ ID NO: 75, SEQ ID NO: 99, SEQ ID NO: 113, SEQ ID NO: 121, SEQ ID NO: 131, SEQ ID NO: 143, SEQ ID NO: 163, SEQ ID NO: 173, SEQ ID NO: 183, SEQ ID NO: 193, and SEQ ID NO: 203.

13. The nucleic acid molecule of claim 11, wherein said heavy chain polypeptide comprises an amino acid sequence that is at least 95% identical to an amino acid sequence selected from the group consisting of: SEQ ID NO: 4, SEQ ID NO: 18, SEQ ID NO: 26, SEQ ID NO: 34, SEQ ID NO: 46, SEQ ID NO: 54, SEQ ID NO: 64, SEQ ID NO: 72, SEQ ID NO: 80, SEQ ID NO: 96, SEQ ID NO: 112, SEQ ID NO: 120, SEQ ID NO: 130, SEQ ID NO: 140, SEQ ID NO: 162, SEQ ID NO: 172, SEQ ID NO: 182, SEQ ID NO: 192, and SEQ ID NO: 202.

14. The nucleic acid molecule of claim 11, wherein said nucleotide sequence encoding the heavy chain polypeptide comprises a nucleotide sequence that is at least 95% identical to a nucleotide sequence selected from the group consisting of: SEQ ID NO: 3, SEQ ID NO: 17, SEQ ID NO: 25, SEQ ID NO: 33, SEQ ID NO: 45, SEQ ID NO: 53, SEQ ID NO: 63, SEQ ID NO: 71, SEQ ID NO: 79, SEQ ID NO: 95, SEQ ID NO: 111, SEQ ID NO: 119, SEQ ID NO: 129, SEQ ID NO: 139, SEQ ID NO: 161, SEQ ID NO: 171, SEQ ID NO: 181, SEQ ID NO: 191, and SEQ ID NO: 201.

15. The nucleic acid molecule of claim 10, wherein said nucleotide sequence encoding the light chain polypeptide comprises a nucleotide sequence encoding a variable light chain region, wherein said variable light chain region comprises an amino acid sequence that is at least 95% identical to an amino acid sequence selected from the group consisting of: SEQ ID NO: 10, SEQ ID NO: 30, SEQ ID NO: 40, SEQ ID NO: 50, SEQ ID NO: 68, SEQ ID NO: 102, SEQ ID NO: 108, SEQ ID NO: 146, and SEQ ID NO: 156.

16. The nucleic acid molecule of claim 15, wherein said nucleotide sequence encoding a variable light chain region comprises a nucleotide sequence that is at least 95% identical to a nucleotide sequence selected from the group consisting of: SEQ ID NO: 9, SEQ ID NO: 29, SEQ ID NO: 39, SEQ ID NO: 49, SEQ ID NO: 67, SEQ ID NO: 101, SEQ ID NO: 107, SEQ ID NO: 145, and SEQ ID NO: 155.

17. The nucleic acid molecule of claim 15, wherein said light chain polypeptide comprises an amino acid sequence that is at least 95% identical to an amino acid sequence selected from the group consisting of: SEQ ID NO: 6, SEQ ID NO: 20, SEQ ID NO: 28, SEQ ID NO: 36, SEQ ID NO: 48, SEQ ID NO: 56, SEQ ID NO: 66, SEQ ID NO: 74, SEQ ID NO: 82, SEQ ID NO: 98, SEQ ID NO: 106, SEQ ID NO: 142, and SEQ ID NO: 154.

18. The nucleic acid molecule of claim 15, wherein said nucleotide sequence encoding the light chain polypeptide comprises a nucleotide sequence that is at least 95% identical to a nucleotide sequence selected from the group consisting of: SEQ ID NO: 5, SEQ ID NO: 19, SEQ ID NO: 27, SEQ ID NO: 35, SEQ ID NO: 47, SEQ ID NO: 55, SEQ ID NO: 65, SEQ ID NO: 73, SEQ ID NO: 81, SEQ ID NO: 97, SEQ ID NO: 105, SEQ ID NO: 141, and SEQ ID NO: 153.

19. A method of treating or preventing Plasmodium parasitic infection, the method comprising administering to the subject at least one selected from the group consisting of:
a) a nucleic acid molecule comprising a nucleotide sequence encoding at least two polypeptides comprising an anti-Plasmodium CSP antibody; and
b) a composition comprising at least two nucleic acid molecules comprising at least two nucleotide sequences encoding at least two polypeptides comprising an anti-Plasmodium CSP antibody.

20. The method of claim 19, wherein said at least two polypeptides comprise a heavy chain polypeptide and a light chain polypeptide, wherein said heavy chain polypeptide comprises a variable heavy chain amino sequence at least 95% identical to an amino acid sequence selected from the group consisting of SEQ ID NO: 8, SEQ ID NO: 22, SEQ ID NO: 38, SEQ ID NO: 58, SEQ ID NO: 76, SEQ ID NO: 100, SEQ ID NO: 114, SEQ ID NO: 122, SEQ ID NO: 132, SEQ ID NO: 144, SEQ ID NO: 164, SEQ ID NO: 174, SEQ ID NO: 184, SEQ ID NO: 194, and SEQ ID NO: 204, encoded by a nucleotide sequence that is at least 90% identical to a nucleotide sequence selected from the group consisting of SEQ ID NO: 7, SEQ ID NO: 21, SEQ ID NO: 37, SEQ ID NO: 57, SEQ ID NO: 75, SEQ ID NO: 99, SEQ ID NO: 113, SEQ ID NO: 121, SEQ ID NO: 131, SEQ ID NO: 143, SEQ ID NO: 163, SEQ ID NO: 173, SEQ ID NO: 183, SEQ ID NO: 193, and SEQ ID NO: 203; and wherein said light chain polypeptide comprises a variable light chain amino sequence at least 95% identical to an amino acid sequence selected from the group consisting of SEQ ID NO: 10, SEQ ID NO: 30, SEQ ID NO: 40, SEQ ID NO: 50, SEQ ID NO: 68, SEQ ID NO: 102, SEQ ID NO: 108, SEQ ID NO: 146, and SEQ ID NO: 156, encoded by a nucleotide sequence that is at least 90% identical to a nucleotide sequence selected from the group consisting of SEQ ID NO: 9, SEQ ID NO: 29, SEQ ID NO: 39, SEQ ID NO: 49, SEQ ID NO: 67, SEQ ID NO: 101, SEQ ID NO: 107, SEQ ID NO: 145, and SEQ ID NO: 155.
